SEQUENCE LISTING (100 sequences)

SEQ ID NO: 1 - DNA, 977 bp, synthetic construct
  misc_feature 1..977: note = Description of Artificial Sequence: Mycobacterium Ag85B
  source 1..977: mol_type = other DNA
atgacagacgtgagccgaaagattcgagcttggggacgccgattgatgatcggcacggcagcggctgtagtccttccgggcctggtggggcttgccgcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 2 - DNA, 858 bp, synthetic construct
  misc_feature 1..858: note = Description of Artificial Sequence: Mycobacterium Ag85B (E. coli optimized)
  source 1..858: mol_type = other DNA
atgtttagccgtcctggcctgccagttgaatacctgcaagttccgagcccgtccatgggtcgtgacattaaggtgcagttccagagcggcggtaacaatagcccggctgtgtacctgctggacggtctgcgtgcgcaggatgattacaacggctgggacatcaataccccggcatttgagtggtattaccagtcgggtctgagcattgtgatgccggttggcggtcaaagcagcttctatagcgattggtacagcccggcatgcggcaaggctggttgccaaacctacaagtgggaaactttcttgaccagcgagctgccgcaatggttgagcgccaaccgtgcggtcaaaccgaccggtagcgctgctattggcctgtccatggccggcagcagcgcgatgatcttggcggcataccatccgcagcagtttatctacgccggtagcctgagcgcattgctggacccgagccaaggcatgggtccgagcctgattggtctggcaatgggtgacgcaggtggttacaaagcggccgatatgtggggcccatctagcgacccggcatgggagcgtaatgacccgacccagcaaattccgaaactggtggcgaataacacgcgcctgtgggtctactgtggcaatggtacgccgaacgagctgggtggcgcgaatatccctgcggagtttctggaaaactttgttcgcagcagcaacctgaaattccaggacgcgtataacgcagccggtggtcacaatgcggttttcaatttcccgccaaatggcactcatagctgggagtactggggtgcgcagttgaacgcaatgaaaggcgatctgcaatcctctctgggtgcgggc

SEQ ID NO: 3 - DNA, 858 bp, synthetic construct
  misc_feature 1..858: note = Description of Artificial Sequence: Mycobacterium Ag85B (human optimized)
  source 1..858: mol_type = other DNA
atgttctccaggcccggcctgcctgtcgagtatctgcaggtcccctccccctccatgggcagagacatcaaggtgcagttccaatccggaggcaacaacagccccgccgtgtatctcctcgacggcctgagggctcaggacgactacaacggctgggacatcaacacccccgccttcgagtggtactaccagtccggactgagcatcgtcatgcccgtgggcggccagagctccttctacagcgactggtatagccctgcctgcggcaaagccggatgccagacctacaagtgggagacctttctgaccagcgaactgccccagtggctgtccgccaatagggccgtcaaacctaccggctccgctgccatcggactcagcatggccggaagctccgctatgatcctggccgcctaccacccccagcaatttatctacgctggcagcctgtccgctctgctggatcctagccaaggcatgggccctagcctcattggcctggccatgggcgatgctggcggctataaggccgccgatatgtggggccctagctccgatcctgcctgggagaggaatgaccccacccagcagatccccaagctggtggccaacaacacaaggctctgggtgtactgcggcaatggcacccccaacgaactgggcggagccaacattcccgccgagttcctggagaacttcgtcaggagcagcaacctgaagttccaggacgcctacaatgccgccggaggccacaacgctgtgttcaacttccctcccaacggcacccacagctgggagtattggggcgctcagctgaacgccatgaaaggcgacctccagagctccctgggagctgga

SEQ ID NO: 4 - protein, 325 aa, synthetic construct
  REGION 1..325: note = Description of Artificial Sequence: Mycobacterium Ag85B
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 5 - protein, 286 aa, synthetic construct
  REGION 1..286: note = Description of Artificial Sequence: Mycobacterium Ag85B (E. coli and human optimized)
MFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 6 - DNA, 288 bp, synthetic construct
  misc_feature 1..288: note = Description of Artificial Sequence: Mycobacterium ESAT-6
  source 1..288: mol_type = other DNA
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 7 - DNA, 282 bp, synthetic construct
  misc_feature 1..282: note = Description of Artificial Sequence: Mycobacterium ESAT-6 (human optimized)
  source 1..282: mol_type = other DNA
accgagcagcagtggaacttcgccggcatcgaagctgccgctagcgccatccaaggcaacgtgaccagcatccacagcctgctggacgagggcaagcagagcctgaccaagctggctgctgcttggggcggatccggaagcgaagcctaccagggcgtgcagcagaagtgggacgccacagccaccgagctgaacaacgccctgcagaacctcgccagaaccatcagcgaggccggacaggctatggccagcacagagggcaatgtgaccggcatgttcgcc

SEQ ID NO: 8 - protein, 94 aa, synthetic construct
  REGION 1..94: note = Description of Artificial Sequence: ESAT-6
TEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 9 - DNA, 687 bp, synthetic construct
  misc_feature 1..687: note = Description of Artificial Sequence: Mycobacterium Rv1980c
  source 1..687: mol_type = other DNA
gtgcgcatcaagatcttcatgctggtcacggctgtcgttttgctctgttgttcgggtgtggccacggccgcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcctag

SEQ ID NO: 10 - DNA, 690 bp, synthetic construct
  misc_feature 1..690: note = Description of Artificial Sequence: Mycobacterium Rv1980c (human optimized)
  source 1..690: mol_type = other DNA
atggtcaggatcaagatcttcatgctcgtgaccgccgtggtgctcctgtgttgttccggcgtggctaccgctgctcccaagacctactgcgaggagctgaagggaaccgacaccggccaggcctgccagatccaaatgagcgaccccgcctacaacatcaacatctccctcccctcctactaccccgatcagaagtccctcgagaactacatcgctcagaccagggacaagttcctgagcgccgccacaagcagcacacccagagaggccccctacgagctgaacatcacctccgccacctaccagtccgctattcctcccagaggcacccaggctgtggtgctcaaggtctaccaaaacgctggcggaacacaccccaccaccacctacaaggccttcgactgggaccaggcctacaggaagcccatcacatacgacaccctgtggcaggctgataccgatcccctgcccgtggtgttccccatcgtgcagggcgagctctccaagcagaccggccagcaagtgagcatcgcccccaatgctggactggaccccgtgaactaccagaacttcgccgtcaccaacgacggcgtgatcttcttcttcaatcccggcgaactgctgcctgaagctgctggccccacccaagtgctggtgcctagaagcgccatcgactccatgctggcctga

SEQ ID NO: 11 - protein, 228 aa, synthetic construct
  REGION 1..228: note = Description of Artificial Sequence: Rv1980c
VRIKIFMLVTAVVLLCCSGVATAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 12 - DNA, 1176 bp, synthetic construct
  misc_feature 1..1176: note = Description of Artificial Sequence: Mycobacterium Rv1039c
  source 1..1176: mol_type = other DNA
atggatttcggagctttaccccctgagatcaactccgcacgcatgtacgccggcgcgggtgcaggaccgatgatggccgccggggccgcatggaacggcctggccgccgagttgggtacgacggccgcgtcgtatgagtcggtgatcacccggctgaccaccgagtcgtggatgggtccggcctcgatggcgatggtcgccgcagcccagccctatctggcttggttgacctacaccgccgaagccgctgcgcatgccggctcgcaggccatggcgtcggcggccgcctacgaggcggcctatgcgatgacagtgccgccggaggtggtcgcggccaaccgggcgctgctggcggccctggtcgcgacgaacgtcctggggatcaacacaccggcaatcatggcgaccgaagccctctatgccgagatgtgggctcaggacgctctggctatgtacggctacgcggccgcttcgggagccgccgggatgctgcaaccgttaagcccgccgtcgcagaccaccaacccgggcgggctggccgcccagtccgccgcggtcggctcggctgccgccaccgccgccgtcaaccaggtgagcgtagcggacctgatcagtagcctgcccaacgcggtgagtgggctcgcctccccagtcacatcggttctcgactcgacggggctgagcggaatcattgccgacatcgacgccctgctcgcgaccccgttcgtggcaaacatcatcaacagcgcagtcaacaccgccgcttggtatgtcaacgccgccatccccaccgcgatattcctagcaaatgccctgaacagtggggcgccggtagcgatcgccgaaggcgccatcgaggctgccgagggtgccgccagtgcggccgccgcggggttggcggactcggtgacgccagcggggctcggcgcaagtttaggcgaggccaccctggtcggccggctgtcagtgccggcggcctggtctacggccgcaccggcgacaaccgccggcgccacagcgctcgaaggcagcggctggaccgtcgccgccgaagaagccggcccagttaccgggatgatgccgggaatggcctcggccgccaagggcaccggtgcctatgccgggccgcggtacggattcaagcccactgtcatgcccaaacaggtcgtcgtgtga

SEQ ID NO: 13 - DNA, 1176 bp, synthetic construct
  misc_feature 1..1176: note = Description of Artificial Sequence: Mycobacterium Rv1039c (human optimized)
  source 1..1176: mol_type = other DNA
atggattttggcgccctgcctcccgagatcaacagcgctaggatgtatgctggcgctggagccggacctatgatggccgctggagccgcctggaatggactggctgccgaactgggcacaacagccgcttcctacgagtccgtgatcaccagactcaccacagagtcctggatgggacctgccagcatggctatggtcgccgctgctcaaccctacctggcctggctgacctatacagctgaagccgctgctcacgccggaagccaagctatggctagcgccgccgcttatgaggccgcttatgccatgaccgtgcctcccgaggtcgtggctgccaacagagctctcctggccgccctcgtggctaccaacgtgctgggaatcaacacccccgctattatggccaccgaggctctgtacgctgagatgtgggcccaggatgccctcgccatgtacggatacgccgctgcttccggagctgctggaatgctgcagcccctgtcccccccttcccagaccaccaaccccggaggactggctgctcaaagcgctgctgtgggatccgctgctgctaccgctgccgtcaatcaggtcagcgtcgccgacctcatctccagcctgcctaacgctgtgagcggactggcctcccctgtcacatccgtgctcgatagcaccggcctgtccggcatcatcgccgacattgatgctctcctcgccaccccctttgtcgccaacatcatcaattccgccgtgaacaccgctgcctggtacgtcaacgctgccattcccaccgccatcttcctcgccaacgccctgaactccggagctcctgtcgccatcgctgagggcgctattgaggctgctgaaggagccgctagcgctgctgctgctggactggctgatagcgtcacccctgctggactcggagctagcctgggagaagccaccctggtcggcagactgtccgtgcctgctgcttggagcaccgctgctcctgctacaaccgctggagctaccgctctggagggatccggatggacagtggctgctgaggaagctggacccgtgaccggaatgatgcctggcatggccagcgctgctaagggaaccggcgcctatgccggacccagatacggattcaagcccaccgtcatgcccaagcaggtcgtcgtctaa

SEQ ID NO: 14 - protein, 391 aa, synthetic construct
  REGION 1..391: note = Description of Artificial Sequence: Rv1039c
MDFGALPPEINSARMYAGAGAGPMMAAGAAWNGLAAELGTTAASYESVITRLTTESWMGPASMAMVAAAQPYLAWLTYTAEAAAHAGSQAMASAAAYEAAYAMTVPPEVVAANRALLAALVATNVLGINTPAIMATEALYAEMWAQDALAMYGYAAASGAAGMLQPLSPPSQTTNPGGLAAQSAAVGSAAATAAVNQVSVADLISSLPNAVSGLASPVTSVLDSTGLSGIIADIDALLATPFVANIINSAVNTAAWYVNAAIPTAIFLANALNSGAPVAIAEGAIEAAEGAASAAAAGLADSVTPAGLGASLGEATLVGRLSVPAAWSTAAPATTAGATALEGSGWTVAAEEAGPVTGMMPGMASAAKGTGAYAGPRYGFKPTVMPKQVVV

SEQ ID NO: 15 - DNA, 1143 bp, synthetic construct
  misc_feature 1..1143: note = Description of Artificial Sequence: Mycobacterium Rv3136
  source 1..1143: mol_type = other DNA
atggatttcgcactgttaccaccggaagtcaactccgcccggatgtacaccggccctggggcaggatcgctgttggctgccgcgggcggctgggattcgctggccgccgagttggccaccacagccgaggcatatggatcggtgctgtccggactggccgccttgcattggcgtggaccggcagcggaatcgatggcggtgacggccgctccctatatcggttggctgtacacgaccgccgaaaagacacagcaaacagcgatccaagccagggcggcagcgctggccttcgagcaagcatacgcaatgaccctgccgccaccggtggtagcggccaaccggatacagctgctagcactgatcgcgacgaacttcttcggccagaacactgcggcgatcgcggccaccgaggcacagtacgccgagatgtgggcccaggacgccgccgcgatgtacggttacgccaccgcctcagcggctgcggccctgctgacaccgttctccccgccgcggcagaccaccaacccggccggcctgaccgctcaggccgccgcggtcagccaggccaccgacccactgtcgctgctgattgagacggtgacccaagcgctgcaagcgctgacgattccgagcttcatccctgaggacttcaccttccttgacgccatattcgctggatatgccacggtaggtgtgacgcaggatgtcgagtcctttgttgccgggaccatcggggccgagagcaacctaggccttttgaacgtcggcgacgagaatcccgcggaggtgacaccgggcgactttgggatcggcgagttggtttccgcgaccagtcccggcggtggggtgtctgcgtcgggtgccggcggtgcggcgagcgtcggcaacacggtgctcgcgagtgtcggccgggcaaactcgattgggcaactatcggtcccaccgagctgggccgcgccctcgacgcgccctgtctcggcattgtcgcccgccggcctgaccacactcccggggaccgacgtggccgagcacgggatgccaggtgtaccgggggtgccagtggcagcagggcgagcctccggcgtcctacctcgatacggggttcggctcacggtgatggcccacccacccgcggcagggtaa

SEQ ID NO: 16 - DNA, 1146 bp, synthetic construct
  misc_feature 1..1146: note = Description of Artificial Sequence: Mycobacterium Rv3136 (E. coli optimized)
  source 1..1146: mol_type = other DNA
atggattttgcgctgctgccgccggaagtgaacagcgcgcgcatgtataccggcccgggcgcgggcagcctgctggcggcggcgggcggctgggatagcctggcggcggaactggcgaccaccgcggaagcgtatggcagcgtgctgagcggcctggcggcgctgcattggcgcggcccggcggcggaaagcatggcggtgaccgcggcgccgtatattggctggctgtataccaccgcggaaaaaacccagcagaccgcgattcaggcgcgcgcggcggcgctggcgtttgaacaggcgtatgcgatgaccctgccgccgccggtggtggcggcgaaccgcattcagctgctggcgctgattgcgaccaacttttttggccagaacaccgcggcgattgcggcgaccgaagcgcagtatgcggaaatgtgggcgcaggatgcggcggcgatgtatggctatgcgaccgcgagcgcggcggcggcgctgctgaccccgtttagcccgccgcgccagaccaccaacccggcgggcctgaccgcgcaggcggcggcggtgagccaggcgaccgatccgctgagcctgctgattgaaaccgtgacccaggcgctgcaggcgctgaccattccgagctttattccggaagattttacctttctggatgcgatttttgcgggctatgcgaccgtgggcgtgacccaggatgtggaaagctttgtggcgggcaccattggcgcggaaagcaacctgggcctgctgaacgtgggcgatgaaaacccggcggaagtgaccccgggcgattttggcattggcgaactggtgagcgcgaccagcccgggcggcggcgtgagcgcgagcggcgcgggcggcgcggcgagcgtgggcaacaccgtgctggcgagcgtgggccgcgcgaacagcattggccagctgagcgtgccgccgagctgggcggcgccgagcacccgcccggtgagcgcgctgagcccggcgggcctgaccaccctgccgggcaccgatgtggcggaacatggcatgccgggcgtgccgggcgtgccggtggcggcgggccgcgcgagcggcgtgctgccgcgctatggcgtgcgcctgaccgtgatggcgcatccgccggcggcgggcgaattt

SEQ ID NO: 17 - DNA, 1143 bp, synthetic construct
  misc_feature 1..1143: note = Description of Artificial Sequence: Mycobacterium Rv3136 (human optimized)
  source 1..1143: mol_type = other DNA
atggatttcgctctgctcccccccgaggtgaatagcgctaggatgtacacaggacccggagctggaagcctcctggctgctgctggaggatgggactccctggctgccgagctcgctacaaccgctgaggcttacggaagcgtgctctccggcctggctgctctccattggagaggccctgctgccgagtccatggctgtcacagccgctccctacattggatggctgtacaccaccgccgagaagacccagcaaaccgctattcaggccagagctgccgccctggccttcgaacaggcctacgctatgacactccccccccctgtcgtggctgccaataggatccagctcctggccctcatcgccaccaacttcttcggccaaaacaccgctgccatcgctgccaccgaagcccagtacgccgaaatgtgggcccaggatgccgctgctatgtacggctatgccacagctagcgctgccgctgctctgctcacacccttcagcccccccaggcaaacaaccaaccctgccggactgacagcccaagctgctgccgtcagccaagctaccgaccccctgagcctcctgatcgaaaccgtgacacaggccctgcaggccctgaccattcccagctttatccccgaggacttcacctttctggacgctatcttcgctggctacgccaccgtgggcgtgacacaagacgtcgagtccttcgtcgccggcacaatcggagccgagtccaacctcggactcctcaacgtcggcgacgaaaatcccgccgaagtgacacctggagacttcggcattggagaactcgtcagcgccacatcccctggcggaggagtgagcgcttccggagctggaggagctgcttccgtgggcaataccgtgctggccagcgtgggaagggccaactccattggccagctcagcgtccccccttcctgggctgccccttccacaaggcctgtgtccgctctcagccctgctggactgaccacactccctggcacagacgtggctgagcatggcatgcccggagtgcctggagtccctgtggctgctggcagagcttccggagtcctccctaggtatggcgtgaggctgacagtgatggctcatccccccgctgccggataa

SEQ ID NO: 18 - protein, 380 aa, synthetic construct
  REGION 1..380: note = Description of Artificial Sequence: Rv3136
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAG

SEQ ID NO: 19 - DNA, 312 bp, synthetic construct
  misc_feature 1..312: note = Description of Artificial Sequence: Mycobacterium Rv3615c
  source 1..312: mol_type = other DNA
atgacggaaaacttgaccgtccagcccgagcgtctcggtgtactggcgtcgcaccatgacaacgcggcggtcgatgcctcctcgggcgtcgaagctgccgctggcctaggcgaatctgtggcgatcactcacggtccgtactgctcacagttcaacgacacgttaaatgtgtacttgactgcccacaatgccctgggctcgtccttgcatacggccggtgtcgatctcgccaaaagtcttcgaattgcggcgaagatatatagcgaggccgacgaagcgtggcgcaaggctatcgacgggttgtttacctga

SEQ ID NO: 20 - DNA, 312 bp, synthetic construct
  misc_feature 1..312: note = Description of Artificial Sequence: Mycobacterium Rv3615c (human optimized)
  source 1..312: mol_type = other DNA
atgaccgagaacctgaccgtgcagcctgagaggctgggagtgctggccagccaccacgacaacgctgccgtggacgcttccagcggagtggaggctgctgctggactgggagagagcgtggccatcacccacggaccctactgcagccagttcaacgacaccctgaacgtgtacctgacagcccacaacgccctgggaagcagcctgcatacagccggcgtggacctggctaagtccctgaggatcgccgccaagatctacagcgaggccgacgaggcctggaggaaagccatcgacggcctgttcacctaa

SEQ ID NO: 21 - protein, 103 aa, synthetic construct
  REGION 1..103: note = Description of Artificial Sequence: Rv3615c
MTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFT

SEQ ID NO: 22 - DNA, 633 bp, synthetic construct
  misc_feature 1..633: note = Description of Artificial Sequence: Mycobacterium Rv1733c
  source 1..633: mol_type = other DNA
atgatcgccacaacccgcgatcgtgaaggagccaccatgatcacgtttaggctgcgcttgccgtgccggacgatactgcgggtgttcagccgcaatccgctggtgcgtgggacggatcgactcgaggcggtcgtcatgctgctggccgtcacggtctcgctgctgactatcccgttcgccgccgcggccggcaccgcagtccaggattcccgcagccacgtctatgcccaccaggcccagacccgccatcccgcaaccgcgaccgtgatcgatcacgagggggtgatcgacagcaacacgaccgccacgtcagcgccgccgcgcacgaagatcaccgtgcctgcccgatgggtcgtgaacggaatagaacgcagcggtgaggtcaacgcgaagccgggaaccaaatccggtgaccgcgtcggcatttgggtcgacagtgccggtcagctggtcgatgaaccagctccgccggcccgtgccattgcggatgcggccctggccgccttgggactctggttgagcgtcgccgcggttgcgggcgccctgctggcgctcactcgggcgattctgatccgcgttcgcaacgccagttggcaacacgacatcgacagcctgttctgcacgcagcggtga

SEQ ID NO: 23 - DNA, 630 bp, synthetic construct
  misc_feature 1..630: note = Description of Artificial Sequence: Mycobacterium Rv1733c (E. coli optimized)
  source 1..630: mol_type = other DNA
atgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgt

SEQ ID NO: 24 - protein, 210 aa, synthetic construct
  REGION 1..210: note = Description of Artificial Sequence: Rv1733c
MIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQR

SEQ ID NO: 25 - DNA, 504 bp, synthetic construct
  misc_feature 1..504: note = Description of Artificial Sequence: Mycobacterium Rv1733c (transmembrane deleted; E. coli optimized)
  source 1..504: mol_type = other DNA
atgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggcccccggggtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgattctagacgcgcaattctgatccgcgttcgcaatgcgagctggcagcacgatattgatagcctgttttgcacccaacgt

SEQ ID NO: 26 - DNA, 489 bp, synthetic construct
  misc_feature 1..489: note = Description of Artificial Sequence: Mycobacterium Rv1733c (transmembrane deleted; human optimized)
  source 1..489: mol_type = other DNA
atcgccaccaccagggacagggaaggcgctaccatgatcaccttcaggctgaggctcccctgcaggaccatcctgagggtgttcagcaggaaccccctggtgaggggcaccgacagactggaagccgtgcaggacagcaggagccacgtgtatgcccaccaggctcagaccaggcaccctgctaccgccaccgtgatcgaccacgagggcgtgatcgactccaacaccaccgccaccagcgctcctcccagaaccaagatcacagtgcccgccaggtgggtggtgaacggcatcgagaggagcggcgaggtgaacgccaagcctggaaccaagagcggcgacagggtgggcatttgggtcgatagcgccggccagctggtggatgaacctgctccccctgccagagccatcgccgatagggccatcctgatcagggtgaggaacgccagctggcagcacgacatcgacagcctgttctgcacccaaagg

SEQ ID NO: 27 - protein, 168 aa, synthetic construct
  REGION 1..168: note = Description of Artificial Sequence: Rv1733c (both transmembrane regions deleted; E. coli optimized)
MIATTRDREGATMITFRLRLPCRTILRVFSRNPLVRGTDRLEAPGVQDSRSHVYAHQAQTRHPATATVIDHEGVIDSNTTATSAPPRTKITVPARWVVNGIERSGEVNAKPGTKSGDRVGIWVDSAGQLVDEPAPPARAIADSRRAILIRVRNASWQHDIDSLFCTQR

SEQ ID NO: 28 - protein, 163 aa, synthetic construct
  REGION 1..163: note = Description of Artificial Sequence: Rv1733c (both transmembrane regions deleted; human optimized)
IATTRDREGATMITFRLRLPCRTILRVFSRNPLVRGTDRLEAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADRAILIRVRNASWQHDIDSLFCTQR

SEQ ID NO: 29 - DNA, 432 bp, synthetic construct
  misc_feature 1..432: note = Description of Artificial Sequence: Mycobacterium Rv2626c
  source 1..432: mol_type = other DNA
atgaccaccgcacgcgacatcatgaacgcaggtgtgacctgtgttggcgaacacgagacgctaaccgctgccgctcaatacatgcgtgagcacgacatcggcgcgttgccgatctgcggggacgacgaccggctgcacggcatgctcaccgaccgcgacattgtgatcaaaggcctggctgcgggcctagacccgaataccgccacggctggcgagttggcccgggacagcatctactacgtcgatgcgaacgcaagcatccaggagatgctcaacgtcatggaagaacatcaggtccgccgtgttccggtcatctcagagcaccgcttggtcggaatcgtcaccgaagccgacatcgcccgacacctgcccgagcacgccattgtgcagttcgtcaaggcaatctgctcgcccatggccctcgccagctag

SEQ ID NO: 30 - DNA, 428 bp, synthetic construct
  misc_feature 1..428: note = Description of Artificial Sequence: Mycobacterium Rv2626c (E. coli optimized)
  source 1..428: mol_type = other DNA
atgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtc

SEQ ID NO: 31 - DNA, 426 bp, synthetic construct
  misc_feature 1..426: note = Description of Artificial Sequence: Mycobacterium Rv2626c (human optimized)
  source 1..426: mol_type = other DNA
acaacagccagggacatcatgaacgccggcgtgacctgcgtgggagagcatgaaaccctcaccgccgccgcccaatacatgagggagcacgacatcggcgccctgcccatctgtggagacgacgacaggctgcacggcatgctgaccgacagggacatcgtgatcaagggcctggctgccggcctcgatcctaacaccgctacagccggcgagctggccagagacagcatctactacgtggacgccaacgccagcatccaggagatgctcaacgtgatggaggagcaccaggtgagaagggtgcctgtgatcagcgagcacaggctggtgggcatcgtgaccgaggccgatatcgctaggcacctgcccgagcacgccatcgtgcagttcgtgaaggccatctgcagccccatggctctggccagc

SEQ ID NO: 32 - protein, 143 aa, synthetic construct
  REGION 1..143: note = Description of Artificial Sequence: Rv2626c
MTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALAS

SEQ ID NO: 33 - DNA, 300 bp, synthetic construct
  misc_feature 1..300: note = Description of Artificial Sequence: Mycobacterium Rv3407
  source 1..300: mol_type = other DNA
atgcgtgctaccgttgggcttgtggaggcaatcggaatccgagaactaagacagcacgcatcgcgatacctcgcccgggttgaagccggcgaggaacttggcgtcaccaacaaaggaagacttgtggcccgactcatcccggtgcaggccgcggagcgttctcgcgaagccctgattgaatcaggtgtcctgattccggctcgtcgtccacaaaaccttctcgacgtcaccgccgaaccggcgcgcggccgcaagcgcaccctgtccgatgttctcaacgaaatgcgcgacgagcagtga

SEQ ID NO: 34 - DNA, 297 bp, synthetic construct
  misc_feature 1..297: note = Description of Artificial Sequence: Mycobacterium Rv3407 (E. coli optimized)
  source 1..297: mol_type = other DNA
atgcgtgcgactgtgggtctggttgaggcgattggcattcgcgagctgcgccaacatgccagccgttacttggctcgtgtcgaggcgggtgaagaactgggcgtgacgaataagggtcgtctggtcgcccgtctgattccggttcaggcagctgagcgttctcgcgaggcgctgattgaatccggcgtcctgatcccggctcgccgtccgcaaaacctgctggacgtgacggcggagccagctcgtggtcgcaaacgcacgctgtctgatgtcctgaacgaaatgcgcgacgagcag

SEQ ID NO: 35 - DNA, 297 bp, synthetic construct
  misc_feature 1..297: note = Description of Artificial Sequence: Mycobacterium Rv3407 (human optimized)
  source 1..297: mol_type = other DNA
atgagggcgaccgtcgggctggtggaggcgataggtatccgggagttgcgacagcacgcatcacgatatctggcacgggtggaagctggggaggaactgggcgtgaccaacaaggggcggctggtcgcgaggctgatccccgtgcaggccgccgagcggtcccgcgaagccctcatcgagtctggggtgctcattccagcacgcaggccgcaaaatctcctggacgtcactgcggagcccgccagaggcagaaagaggacgctgagtgacgtgctgaacgagatgagggacgaacag

SEQ ID NO: 36 - protein, 99 aa, synthetic construct
  REGION 1..99: note = Description of Artificial Sequence: Rv3407
MRATVGLVEAIGIRELRQHASRYLARVEAGEELGVTNKGRLVARLIPVQAAERSREALIESGVLIPARRPQNLLDVTAEPARGRKRTLSDVLNEMRDEQ

SEQ ID NO: 37 - DNA, 363 bp, synthetic construct
  misc_feature 1..363: note = Description of Artificial Sequence: Mycobacterium Rv2628c
  source 1..363: mol_type = other DNA
atgtccacgcaacgaccgaggcactccggtattcgggctgttggcccctacgcatgggccggccgatgtggtcggataggcaggtggggggtgcaccaggaggcgatgatgaatctagcgatatggcacccgcgcaaggtgcaatccgccaccatctatcaggtgaccgatcgctcgcacgacgggcgcacagcacgggtgcctggtgacgagatcactagcaccgtgtccggttggttgtcggagttgggcacccaaagcccgttggccgatgagcttgcgcgtgcggtgcggatcggcgactggcccgctgcgtacgcaatcggtgagcacctgtccgttgagattgccgttgcggtctaa

SEQ ID NO: 38 - DNA, 363 bp, synthetic construct
  misc_feature 1..363: note = Description of Artificial Sequence: Mycobacterium Rv2628c (human optimized)
  source 1..363: mol_type = other DNA
atgagcacccagagacccaggcacagcggcattagggccgtgggaccttatgcttgggccggcagatgcggaaggatcggcagatggggcgtgcaccaagaggccatgatgaacctggccatctggcaccccaggaaggtgcagagcgccaccatctaccaggtgaccgacaggagccatgacggaaggaccgccagagtgcccggcgatgagatcaccagcaccgtgagcggctggctgagcgaactgggcacccaatcccccctggctgatgaactggccagggctgtgaggatcggcgattggcctgccgcctatgccatcggcgagcatctgagcgtggagatcgccgtggccgtgtaa

SEQ ID NO: 39 - protein, 120 aa, synthetic construct
  REGION 1..120: note = Description of Artificial Sequence: Rv2628c
MSTQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAV

SEQ ID NO: 40 - DNA, 1089 bp, synthetic construct
  misc_feature 1..1089: note = Description of Artificial Sequence: Mycobacterium Rv1009
  source 1..1089: mol_type = other DNA
atgttgcgcctggtagtcggtgcgctgctgctggtgttggcgttcgccggtggctatgcggtcgccgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 41 - DNA, 1023 bp, synthetic construct
  misc_feature 1..1023: note = Description of Artificial Sequence: Mycobacterium Rv1009 (signal sequence deleted)
  source 1..1023: mol_type = other DNA
gcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 42 - protein, 362 aa, synthetic construct
  REGION 1..362: note = Description of Artificial Sequence: RpfB (mycobacterial sequence)
MLRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 43 - protein, 340 aa, synthetic construct
  REGION 1..340: note = Description of Artificial Sequence: RpfB (mycobacterial sequence; signal sequence deleted)
ACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 44 - DNA, 465 bp, synthetic construct
  misc_feature 1..465: note = Description of Artificial Sequence: Mycobacterium Rv2389c
  source 1..465: mol_type = other DNA
atgacaccgggtttgcttactactgcgggtgctggccgaccacgtgacaggtgcgccaggatcgtatgcacggtgttcatcgaaaccgccgttgtcgcgaccatgtttgtcgcgttgttgggtctgtccaccatcagctcgaaagccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgattga

SEQ ID NO: 45 - DNA, 354 bp, synthetic construct
  misc_feature 1..354: note = Description of Artificial Sequence: Mycobacterium Rv2389c (E. coli optimized; leader sequence deleted)
  source 1..354: mol_type = other DNA
aagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgac

SEQ ID NO: 46 - DNA, 465 bp, synthetic construct
  misc_feature 1..465: note = Description of Artificial Sequence: Mycobacterium Rv2389c (human optimized; leader sequence present)
  source 1..465: mol_type = other DNA
acccccggactcctcaccacagctggagctggcaggcccagagacagatgcgccaggatcgtgtgcaccgtgttcatcgagaccgccgtggtggctaccatgttcgtggccctgctgggcctgagcaccatcagcagcaaggccgacgacatcgactgggacgccatcgcccagtgtgaatccggcggaaactgggccgccaataccggcaatggcctgtacggcggcctgcagatcagccaggctacctgggactccaacggaggagtgggaagccctgccgctgcttcccctcagcagcagatcgaggtggccgacaacatcatgaagacccaaggccctggcgcctggcctaagtgttccagctgtagccagggcgatgctcctctgggcagcctgacccacatcctgacctttctcgccgccgagacaggcggatgtagcggaagcagggacgactaatga

SEQ ID NO: 47 - protein, 116 aa, synthetic construct
  REGION 1..116: note = Description of Artificial Sequence: RpfD (E. coli optimized)
LLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 48 - protein, 153 aa, synthetic construct
  REGION 1..153: note = Description of Artificial Sequence: RpfD (mycobacterial sequence and human optimized)
TPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 49 - DNA, 519 bp, synthetic construct
  misc_feature 1..519: note = Description of Artificial Sequence: Mycobacterium Rv2450c
  source 1..519: mol_type = other DNA
ttgaagaacgcccgtacgacgctcatcgccgccgcgattgccgggacgttggtgaccacgtcaccagccggtatcgccaatgccgacgacgcgggcttggacccaaacgccgcagccggcccggatgccgtgggctttgacccgaacctgccgccggccccggacgctgcacccgtcgatactccgccggctccggaggacgcgggctttgatcccaacctccccccgccgctggccccggacttcctgtccccgcctgcggaggaagcgcctcccgtgcccgtggcctacagcgtgaactgggacgcgatcgcgcagtgcgagtccggtggaaactggtcgatcaacaccggtaacggttactacggcggcctgcggttcaccgccggcacctggcgtgccaacggtggctcggggtccgcggccaacgcgagccgggaggagcagatccgggtggctgagaacgtgctgcgttcgcagggtatccgcgcctggccggtctgcggccgccgcggctga

SEQ ID NO: 50 - protein, 172 aa, synthetic construct
  REGION 1..172: note = Description of Artificial Sequence: RpfE
LKNARTTLIAAAIAGTLVTTSPAGIANADDAGLDPNAAAGPDAVGFDPNLPPAPDAAPVDTPPAPEDAGFDPNLPPPLAPDFLSPPAEEAPPVPVAYSVNWDAIAQCESGGNWSINTGNGYYGGLRFTAGTWRANGGSGSAANASREEQIRVAENVLRSQGIRAWPVCGRRG

SEQ ID NO: 51 - DNA, 1584 bp, synthetic construct
  misc_feature 1..1584: note = Description of Artificial Sequence: Mycobacterium ESAT6-Rv1733c- Rv2626c-RpfD fusion protein (E. coli optimized)
  source 1..1584: mol_type = other DNA
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggcccccggggtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgattctagacgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctaagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgac

SEQ ID NO: 52 - DNA, 1689 bp, synthetic construct
  misc_feature 1..1689: note = Description of Artificial Sequence: Mycobacterium ESAT6-Rv1733c- Rv2626c-RpfD fusion protein (human optimized)
  source 1..1689: mol_type = other DNA
atgaccgagcagcagtggaacttcgccggcatcgaagctgccgctagcgccatccaaggcaacgtgaccagcatccacagcctgctggacgagggcaagcagagcctgaccaagctggctgctgcttggggcggatccggaagcgaagcctaccagggcgtgcagcagaagtgggacgccacagccaccgagctgaacaacgccctgcagaacctcgccagaaccatcagcgaggccggacaggctatggccagcacagagggcaatgtgaccggcatgttcgccttcgaaatcgccaccaccagggacagggaaggcgctaccatgatcaccttcaggctgaggctcccctgcaggaccatcctgagggtgttcagcaggaaccccctggtgaggggcaccgacagactggaagccgtgcaggacagcaggagccacgtgtatgcccaccaggctcagaccaggcaccctgctaccgccaccgtgatcgaccacgagggcgtgatcgactccaacaccaccgccaccagcgctcctcccagaaccaagatcacagtgcccgccaggtgggtggtgaacggcatcgagaggagcggcgaggtgaacgccaagcctggaaccaagagcggcgacagggtgggcatttgggtcgatagcgccggccagctggtggatgaacctgctccccctgccagagccatcgccgatagggccatcctgatcagggtgaggaacgccagctggcagcacgacatcgacagcctgttctgcacccaaaggcgatcgacaacagccagggacatcatgaacgccggcgtgacctgcgtgggagagcatgaaaccctcaccgccgccgcccaatacatgagggagcacgacatcggcgccctgcccatctgtggagacgacgacaggctgcacggcatgctgaccgacagggacatcgtgatcaagggcctggctgccggcctcgatcctaacaccgctacagccggcgagctggccagagacagcatctactacgtggacgccaacgccagcatccaggagatgctcaacgtgatggaggagcaccaggtgagaagggtgcctgtgatcagcgagcacaggctggtgggcatcgtgaccgaggccgatatcgctaggcacctgcccgagcacgccatcgtgcagttcgtgaaggccatctgcagccccatggctctggccagcggcgcgcccacccccggactcctcaccacagctggagctggcaggcccagagacagatgcgccaggatcgtgtgcaccgtgttcatcgagaccgccgtggtggctaccatgttcgtggccctgctgggcctgagcaccatcagcagcaaggccgacgacatcgactgggacgccatcgcccagtgtgaatccggcggaaactgggccgccaataccggcaatggcctgtacggcggcctgcagatcagccaggctacctgggactccaacggaggagtgggaagccctgccgctgcttcccctcagcagcagatcgaggtggccgacaacatcatgaagacccaaggccctggcgcctggcctaagtgttccagctgtagccagggcgatgctcctctgggcagcctgacccacatcctgacctttctcgccgccgagacaggcggatgtagcggaagcagggacgactaatgatag

SEQ ID NO: 53 - protein, 528 aa, synthetic construct
  REGION 1..528: note = Description of Artificial Sequence: ESAT6-Rv1733c-Rv2626c-RpfD (E. coli optimized)
MTEQQWNFAGIEAAASAIQGNVTSIHSLLDEGKQSLTKLAAAWGGSGSEAYQGVQQKWDATATELNNALQNLARTISEAGQAMASTEGNVTGMFAEFMIATTRDREGATMITFRLRLPCRTILRVFSRNPLVRGTDRLEAPGVQDSRSHVYAHQAQTRHPATATVIDHEGVIDSNTTATSAPPRTKITVPARWVVNGIERSGEVNAKPGTKSGDRVGIWVDSAGQLVDEPAPPARAIADSRRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASKLLLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 54 - protein, 559 aa, synthetic construct
  REGION 1..559: note = Description of Artificial Sequence: ESAT6-Rv1733c-Rv2626c-RpfD (human optimized)
MTEQQWNFAGIEAAASAIQGNVTSIHSLLDEGKQSLTKLAAAWGGSGSEAYQGVQQKWDATATELNNALNLARTISEAGQAMASTEGNVTGMFAFEIATTRDREGATMITFRLRLPCRTILRVFSRNPLVRGTDRLEAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADRAILIRVRNASWQHDIDSLFCTQRRSTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASGAPTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 55 - DNA, 2421 bp, synthetic construct
  misc_feature 1..2421: note = Description of Artificial Sequence: Mycobacterium ESAT6-Rv1733c- Rv2626c-RpfB fusion protein
  source 1..2421: mol_type = other DNA
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcaaagcttgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 56 - protein, 806 aa, synthetic construct
  REGION 1..806: note = Description of Artificial Sequence: ESAT6-Rv1733c-Rv2626c-RpfB
MTEQQWNFAGIEAAASAIQGNVTSIHSLLDEGKQSLTKLAAAWGGSGSEAYQGVQQKWDATATELNNALQNLARTISEAGQAMASTEGNVTGMFAEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNAKLACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 57 - DNA, 2430 bp, synthetic construct
  misc_feature 1..2430: note = Description of Artificial Sequence: Mycobacterium RpfB-ESAT6- Rv1733c-Rv2626c fusion protein
  source 1..2430: mol_type = other DNA
atgaagcttgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgcggatccatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcatga

SEQ ID NO: 58 - protein, 809 aa, synthetic construct
  REGION 1..809: note = Description of Artificial Sequence: RpfB-ESAT6-Rv1733c-Rv2626c
MKLACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGARGSMTEQQWNFAGIEAAASAIQGNVTSIHSLLDEGKQSLTKLAAAWGGSGSEAYQGVQQKWDATATELNNALQNLARTISEAGQAMASTEGNVTGMFAEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNA

SEQ ID NO: 59 - DNA, 2448 bp, synthetic construct
  misc_feature 1..2448: note = Description of Artificial Sequence: Mycobacterium Ag85B-ESAT6- Rv1733c-Rv2626c-RpfD fusion protein (E. coli optimized)
  source 1..2448: mol_type = other DNA
atgtttagccgtcctggcctgccagttgaatacctgcaagttccgagcccgtccatgggtcgtgacattaaggtgcagttccagagcggcggtaacaatagcccggctgtgtacctgctggacggtctgcgtgcgcaggatgattacaacggctgggacatcaataccccggcatttgagtggtattaccagtcgggtctgagcattgtgatgccggttggcggtcaaagcagcttctatagcgattggtacagcccggcatgcggcaaggctggttgccaaacctacaagtgggaaactttcttgaccagcgagctgccgcaatggttgagcgccaaccgtgcggtcaaaccgaccggtagcgctgctattggcctgtccatggccggcagcagcgcgatgatcttggcggcataccatccgcagcagtttatctacgccggtagcctgagcgcattgctggacccgagccaaggcatgggtccgagcctgattggtctggcaatgggtgacgcaggtggttacaaagcggccgatatgtggggcccatctagcgacccggcatgggagcgtaatgacccgacccagcaaattccgaaactggtggcgaataacacgcgcctgtgggtctactgtggcaatggtacgccgaacgagctgggtggcgcgaatatccctgcggagtttctggaaaactttgttcgcagcagcaacctgaaattccaggacgcgtataacgcagccggtggtcacaatgcggttttcaatttcccgccaaatggcactcatagctgggagtactggggtgcgcagttgaacgcaatgaaaggcgatctgcaatcctctctgggtgcgggcggatccatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggcccccggggtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgattctagacgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctaagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgac

SEQ ID NO: 60 - DNA, 2550 bp, synthetic construct
  misc_feature 1..2550: note = Description of Artificial Sequence: Mycobacterium Ag85B-ESAT6- Rv1733c-Rv2626c-RpfD fusion protein (human optimized)
  source 1..2550: mol_type = other DNA
atgttctccaggcccggcctgcctgtcgagtatctgcaggtcccctccccctccatgggcagagacatcaaggtgcagttccaatccggaggcaacaacagccccgccgtgtatctcctcgacggcctgagggctcaggacgactacaacggctgggacatcaacacccccgccttcgagtggtactaccagtccggactgagcatcgtcatgcccgtgggcggccagagctccttctacagcgactggtatagccctgcctgcggcaaagccggatgccagacctacaagtgggagacctttctgaccagcgaactgccccagtggctgtccgccaatagggccgtcaaacctaccggctccgctgccatcggactcagcatggccggaagctccgctatgatcctggccgcctaccacccccagcaatttatctacgctggcagcctgtccgctctgctggatcctagccaaggcatgggccctagcctcattggcctggccatgggcgatgctggcggctataaggccgccgatatgtggggccctagctccgatcctgcctgggagaggaatgaccccacccagcagatccccaagctggtggccaacaacacaaggctctgggtgtactgcggcaatggcacccccaacgaactgggcggagccaacattcccgccgagttcctggagaacttcgtcaggagcagcaacctgaagttccaggacgcctacaatgccgccggaggccacaacgctgtgttcaacttccctcccaacggcacccacagctgggagtattggggcgctcagctgaacgccatgaaaggcgacctccagagctccctgggagctggacccgggaccgagcagcagtggaacttcgccggcatcgaagctgccgctagcgccatccaaggcaacgtgaccagcatccacagcctgctggacgagggcaagcagagcctgaccaagctggctgctgcttggggcggatccggaagcgaagcctaccagggcgtgcagcagaagtgggacgccacagccaccgagctgaacaacgccctgcagaacctcgccagaaccatcagcgaggccggacaggctatggccagcacagagggcaatgtgaccggcatgttcgccttcgaaatcgccaccaccagggacagggaaggcgctaccatgatcaccttcaggctgaggctcccctgcaggaccatcctgagggtgttcagcaggaaccccctggtgaggggcaccgacagactggaagccgtgcaggacagcaggagccacgtgtatgcccaccaggctcagaccaggcaccctgctaccgccaccgtgatcgaccacgagggcgtgatcgactccaacaccaccgccaccagcgctcctcccagaaccaagatcacagtgcccgccaggtgggtggtgaacggcatcgagaggagcggcgaggtgaacgccaagcctggaaccaagagcggcgacagggtgggcatttgggtcgatagcgccggccagctggtggatgaacctgctccccctgccagagccatcgccgatagggccatcctgatcagggtgaggaacgccagctggcagcacgacatcgacagcctgttctgcacccaaaggcgatcgacaacagccagggacatcatgaacgccggcgtgacctgcgtgggagagcatgaaaccctcaccgccgccgcccaatacatgagggagcacgacatcggcgccctgcccatctgtggagacgacgacaggctgcacggcatgctgaccgacagggacatcgtgatcaagggcctggctgccggcctcgatcctaacaccgctacagccggcgagctggccagagacagcatctactacgtggacgccaacgccagcatccaggagatgctcaacgtgatggaggagcaccaggtgagaagggtgcctgtgatcagcgagcacaggctggtgggcatcgtgaccgaggccgatatcgctaggcacctgcccgagcacgccatcgtgcagttcgtgaaggccatctgcagccccatggctctggccagcggcgcgcccacccccggactcctcaccacagctggagctggcaggcccagagacagatgcgccaggatcgtgtgcaccgtgttcatcgagaccgccgtggtggctaccatgttcgtggccctgctgggcctgagcaccatcagcagcaaggccgacgacatcgactgggacgccatcgcccagtgtgaatccggcggaaactgggccgccaataccggcaatggcctgtacggcggcctgcagatcagccaggctacctgggactccaacggaggagtgggaagccctgccgctgcttcccctcagcagcagatcgaggtggccgacaacatcatgaagacccaaggccctggcgcctggcctaagtgttccagctgtagccagggcgatgctcctctgggcagcctgacccacatcctgacctttctcgccgccgagacaggcggatgtagcggaagcagggacgactaatgatag

SEQ ID NO: 61 - protein, 816 aa, synthetic construct
  REGION 1..816: note = Description of Artificial Sequence: Ag85B-ESAT6-Rv1733c-Rv2626c- RpfD (E. coli optimized)
MFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGGSMTEQQWNFAGIEAAASAIQGNVTSIHSLLDEGKQSLTKLAAAWGGSGSEAYQGVQQKWDATATELNNALQNLARTISEAGQAMASTEGNVTGMFAEFMIATTRDREGATMITFRLRLPCRTILRVFSRNPLVRGTDRLEAPGVQDSRSHVYAHQAQTRHPATATVIDHEGVIDSNTTATSAPPRTKITVPARWVVNGIERSGEVNAKPGTKSGDRVGIWVDSAGQLVDEPAPPARAIADSRRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASKLLLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 62 - protein, 847 aa, synthetic construct
  REGION 1..847: note = Description of Artificial Sequence: Ag85B-ESAT6-Rv1733c-Rv2626c- RpfD (human optimized)
MFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGPGTEQQWNFAGIEAAASAIQGNVTSIHSLLDEGKQSLTKLAAAWGGSGSEAYQGVQQKWDATATELNNALQNLARTISEAGQAMASTEGNVTGMFAFEIATTRDREGATMITFRLRLPCRTILRVFSRNPLVRGTDRLEAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADRAILIRVRNASWQHDIDSLFCTQRRSTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASGAPTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 63 - DNA, 2499 bp, synthetic construct
  misc_feature 1..2499: note = Description of Artificial Sequence: Mycobacterium PPE51-Rv1733c- Rv2628c-RpfD fusion protein
  source 1..2499: mol_type = other DNA
atggattttgcgctgctgccgccggaagtgaacagcgcgcgcatgtataccggcccgggcgcgggcagcctgctggcggcggcgggcggctgggatagcctggcggcggaactggcgaccaccgcggaagcgtatggcagcgtgctgagcggcctggcggcgctgcattggcgcggcccggcggcggaaagcatggcggtgaccgcggcgccgtatattggctggctgtataccaccgcggaaaaaacccagcagaccgcgattcaggcgcgcgcggcggcgctggcgtttgaacaggcgtatgcgatgaccctgccgccgccggtggtggcggcgaaccgcattcagctgctggcgctgattgcgaccaacttttttggccagaacaccgcggcgattgcggcgaccgaagcgcagtatgcggaaatgtgggcgcaggatgcggcggcgatgtatggctatgcgaccgcgagcgcggcggcggcgctgctgaccccgtttagcccgccgcgccagaccaccaacccggcgggcctgaccgcgcaggcggcggcggtgagccaggcgaccgatccgctgagcctgctgattgaaaccgtgacccaggcgctgcaggcgctgaccattccgagctttattccggaagattttacctttctggatgcgatttttgcgggctatgcgaccgtgggcgtgacccaggatgtggaaagctttgtggcgggcaccattggcgcggaaagcaacctgggcctgctgaacgtgggcgatgaaaacccggcggaagtgaccccgggcgattttggcattggcgaactggtgagcgcgaccagcccgggcggcggcgtgagcgcgagcggcgcgggcggcgcggcgagcgtgggcaacaccgtgctggcgagcgtgggccgcgcgaacagcattggccagctgagcgtgccgccgagctgggcggcgccgagcacccgcccggtgagcgcgctgagcccggcgggcctgaccaccctgccgggcaccgatgtggcggaacatggcatgccgggcgtgccgggcgtgccggtggcggcgggccgcgcgagcggcgtgctgccgcgctatggcgtgcgcctgaccgtgatggcgcatccgccggcggcgggcgaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgtccacgcaacgaccgaggcactccggtattcgggctgttggcccctacgcatgggccggccgatgtggtcggataggcaggtggggggtgcaccaggaggcgatgatgaatctagcgatatggcacccgcgcaaggtgcaatccgccaccatctatcaggtgaccgatcgctcgcacgacgggcgcacagcacgggtgcctggtgacgagatcactagcaccgtgtccggttggttgtcggagttgggcacccaaagcccgttggccgatgagcttgcgcgtgcggtgcggatcggcgactggcccgctgcgtacgcaatcggtgagcacctgtccgttgagattgccgttgcggtcaagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgactga

SEQ ID NO: 64 - protein, 834 aa, synthetic construct
  REGION 1..834: note = Description of Artificial Sequence: PPE51-Rv1733c-Rv2628c-RpfD
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGEFEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMSTQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAVKLLLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 65 - DNA, 3171 bp, synthetic construct
  misc_feature 1..3171: note = Description of Artificial Sequence: Mycobacterium PPE51-Rv1733c- Rv2628c-RpfB fusion protein
  source 1..3171: mol_type = other DNA
atggattttgcgctgctgccgccggaagtgaacagcgcgcgcatgtataccggcccgggcgcgggcagcctgctggcggcggcgggcggctgggatagcctggcggcggaactggcgaccaccgcggaagcgtatggcagcgtgctgagcggcctggcggcgctgcattggcgcggcccggcggcggaaagcatggcggtgaccgcggcgccgtatattggctggctgtataccaccgcggaaaaaacccagcagaccgcgattcaggcgcgcgcggcggcgctggcgtttgaacaggcgtatgcgatgaccctgccgccgccggtggtggcggcgaaccgcattcagctgctggcgctgattgcgaccaacttttttggccagaacaccgcggcgattgcggcgaccgaagcgcagtatgcggaaatgtgggcgcaggatgcggcggcgatgtatggctatgcgaccgcgagcgcggcggcggcgctgctgaccccgtttagcccgccgcgccagaccaccaacccggcgggcctgaccgcgcaggcggcggcggtgagccaggcgaccgatccgctgagcctgctgattgaaaccgtgacccaggcgctgcaggcgctgaccattccgagctttattccggaagattttacctttctggatgcgatttttgcgggctatgcgaccgtgggcgtgacccaggatgtggaaagctttgtggcgggcaccattggcgcggaaagcaacctgggcctgctgaacgtgggcgatgaaaacccggcggaagtgaccccgggcgattttggcattggcgaactggtgagcgcgaccagcccgggcggcggcgtgagcgcgagcggcgcgggcggcgcggcgagcgtgggcaacaccgtgctggcgagcgtgggccgcgcgaacagcattggccagctgagcgtgccgccgagctgggcggcgccgagcacccgcccggtgagcgcgctgagcccggcgggcctgaccaccctgccgggcaccgatgtggcggaacatggcatgccgggcgtgccgggcgtgccggtggcggcgggccgcgcgagcggcgtgctgccgcgctatggcgtgcgcctgaccgtgatggcgcatccgccggcggcgggcgaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgtccacgcaacgaccgaggcactccggtattcgggctgttggcccctacgcatgggccggccgatgtggtcggataggcaggtggggggtgcaccaggaggcgatgatgaatctagcgatatggcacccgcgcaaggtgcaatccgccaccatctatcaggtgaccgatcgctcgcacgacgggcgcacagcacgggtgcctggtgacgagatcactagcaccgtgtccggttggttgtcggagttgggcacccaaagcccgttggccgatgagcttgcgcgtgcggtgcggatcggcgactggcccgctgcgtacgcaatcggtgagcacctgtccgttgagattgccgttgcggtcaagcttgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 66 - protein, 1056 aa, synthetic construct
  REGION 1..1056: note = Description of Artificial Sequence: PPE51-Rv1733c-Rv2628c-RpfB
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMSTQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAVKLACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 67 - DNA, 2433 bp, synthetic construct
  misc_feature 1..2433: note = Description of Artificial Sequence: Mycobacterium Rv3407-Rv1733c- Rv2626c-RpfB fusion protein
  source 1..2433: mol_type = other DNA
atgcgtgcgactgtgggtctggttgaggcgattggcattcgcgagctgcgccaacatgccagccgttacttggctcgtgtcgaggcgggtgaagaactgggcgtgacgaataagggtcgtctggtcgcccgtctgattccggttcaggcagctgagcgttctcgcgaggcgctgattgaatccggcgtcctgatcccggctcgccgtccgcaaaacctgctggacgtgacggcggagccagctcgtggtcgcaaacgcacgctgtctgatgtcctgaacgaaatgcgcgacgagcaggaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcaaagcttgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 68 - protein, 810 aa, synthetic construct
  REGION 1..810: note = Description of Artificial Sequence: Rv3407-Rv1733c-Rv2626c-RpfB
MRATVGLVEAIGIRELRQHASRYLARVEAGEELGVTNKGRLVARLIPVQAAERSREALIESGVLIPARRPQNLLDVTAEPARGRKRTLSDVLNEMRDEQEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNAKLACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 69 - DNA, 1761 bp, synthetic construct
  misc_feature 1..1761: note = Description of Artificial Sequence: Mycobacterium Rv3407-Rv1733c- Rv2626c-RpfD fusion protein
  source 1..1761: mol_type = other DNA
atgcgtgcgactgtgggtctggttgaggcgattggcattcgcgagctgcgccaacatgccagccgttacttggctcgtgtcgaggcgggtgaagaactgggcgtgacgaataagggtcgtctggtcgcccgtctgattccggttcaggcagctgagcgttctcgcgaggcgctgattgaatccggcgtcctgatcccggctcgccgtccgcaaaacctgctggacgtgacggcggagccagctcgtggtcgcaaacgcacgctgtctgatgtcctgaacgaaatgcgcgacgagcaggaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcaaagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgactga

SEQ ID NO: 70 - protein, 586 aa, synthetic construct
  REGION 1..586: note = Description of Artificial Sequence: Rv3407-Rv1733c-Rv2626c-RpfD
MRATVGLVEAIGIRELRQHASRYLARVEAGEELGVTNKGRLVARLIPVQAAERSREALIESGVLIPARRPQNLLDVTAEPARGRKRTLSDVLNEMRDEQEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNAKLLLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 71 - DNA, 2895 bp, synthetic construct
  misc_feature 1..2895: note = Description of Artificial Sequence: Mycobacterium PPE51-Rv1733c- Rv2626c-RpfD fusion protein
  source 1..2895: mol_type = other DNA
atggattttgcgctgctgccgccggaagtgaacagcgcgcgcatgtataccggcccgggcgcgggcagcctgctggcggcggcgggcggctgggatagcctggcggcggaactggcgaccaccgcggaagcgtatggcagcgtgctgagcggcctggcggcgctgcattggcgcggcccggcggcggaaagcatggcggtgaccgcggcgccgtatattggctggctgtataccaccgcggaaaaaacccagcagaccgcgattcaggcgcgcgcggcggcgctggcgtttgaacaggcgtatgcgatgaccctgccgccgccggtggtggcggcgaaccgcattcagctgctggcgctgattgcgaccaacttttttggccagaacaccgcggcgattgcggcgaccgaagcgcagtatgcggaaatgtgggcgcaggatgcggcggcgatgtatggctatgcgaccgcgagcgcggcggcggcgctgctgaccccgtttagcccgccgcgccagaccaccaacccggcgggcctgaccgcgcaggcggcggcggtgagccaggcgaccgatccgctgagcctgctgattgaaaccgtgacccaggcgctgcaggcgctgaccattccgagctttattccggaagattttacctttctggatgcgatttttgcgggctatgcgaccgtgggcgtgacccaggatgtggaaagctttgtggcgggcaccattggcgcggaaagcaacctgggcctgctgaacgtgggcgatgaaaacccggcggaagtgaccccgggcgattttggcattggcgaactggtgagcgcgaccagcccgggcggcggcgtgagcgcgagcggcgcgggcggcgcggcgagcgtgggcaacaccgtgctggcgagcgtgggccgcgcgaacagcattggccagctgagcgtgccgccgagctgggcggcgccgagcacccgcccggtgagcgcgctgagcccggcgggcctgaccaccctgccgggcaccgatgtggcggaacatggcatgccgggcgtgccgggcgtgccggtggcggcgggccgcgcgagcggcgtgctgccgcgctatggcgtgcgcctgaccgtgatggcgcatccgccggcggcgggcgaatttatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcaaagcttttgctgggcctgagcaccattagcagcaaagcggatgacatcgactgggatgcgattgcgcagtgtgagagcggtggcaattgggcagcgaataccggcaatggcctgtacggcggtctgcagatctcccaggcgacgtgggacagcaatggtggcgtcggcagcccggctgccgcgtccccacaacaacagatcgaggtggcagataacattatgaaaacgcagggtccgggtgcttggccaaaatgctccagctgcagccagggtgacgcaccgctgggcagcctgacccacattctgacgttcctggcagcggaaaccggtggttgtagcggtagccgcgatgactga

SEQ ID NO: 72 - protein, 870 aa, synthetic construct
  REGION 1..870: note = Description of Artificial Sequence: PPE51-Rv1733c-Rv2626c-RpfD
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNAKLLLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDKMK

SEQ ID NO: 73 - DNA, 3567 bp, synthetic construct
  misc_feature 1..3567: note = Description of Artificial Sequence: Mycobacterium PPE51-Rv1733c- Rv2626c-RpfB fusion protein
  source 1..3567: mol_type = other DNA
atggattttgcgctgctgccgccggaagtgaacagcgcgcgcatgtataccggcccgggcgcgggcagcctgctggcggcggcgggcggctgggatagcctggcggcggaactggcgaccaccgcggaagcgtatggcagcgtgctgagcggcctggcggcgctgcattggcgcggcccggcggcggaaagcatggcggtgaccgcggcgccgtatattggctggctgtataccaccgcggaaaaaacccagcagaccgcgattcaggcgcgcgcggcggcgctggcgtttgaacaggcgtatgcgatgaccctgccgccgccggtggtggcggcgaaccgcattcagctgctggcgctgattgcgaccaacttttttggccagaacaccgcggcgattgcggcgaccgaagcgcagtatgcggaaatgtgggcgcaggatgcggcggcgatgtatggctatgcgaccgcgagcgcggcggcggcgctgctgaccccgtttagcccgccgcgccagaccaccaacccggcgggcctgaccgcgcaggcggcggcggtgagccaggcgaccgatccgctgagcctgctgattgaaaccgtgacccaggcgctgcaggcgctgaccattccgagctttattccggaagattttacctttctggatgcgatttttgcgggctatgcgaccgtgggcgtgacccaggatgtggaaagctttgtggcgggcaccattggcgcggaaagcaacctgggcctgctgaacgtgggcgatgaaaacccggcggaagtgaccccgggcgattttggcattggcgaactggtgagcgcgaccagcccgggcggcggcgtgagcgcgagcggcgcgggcggcgcggcgagcgtgggcaacaccgtgctggcgagcgtgggccgcgcgaacagcattggccagctgagcgtgccgccgagctgggcggcgccgagcacccgcccggtgagcgcgctgagcccggcgggcctgaccaccctgccgggcaccgatgtggcggaacatggcatgccgggcgtgccgggcgtgccggtggcggcgggccgcgcgagcggcgtgctgccgcgctatggcgtgcgcctgaccgtgatggcgcatccgccggcggcgggcgaatttatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagaattcatgattgcgactacccgtgatcgtgagggcgcgaccatgatcacgttccgtctgcgtctgccgtgtcgcaccattttgcgcgtgttttcgcgtaacccgctggtccgcggtaccgaccgtctggaggccgttgtcatgctgctggcggttaccgtgagcctgctgacgatcccattcgcagcggcagctggcacggccgtccaagacagccgtagccatgtgtatgctcaccaggctcaaacccgtcacccggctactgccactgttatcgatcacgaaggcgtgattgactccaataccacggcaacctccgcaccgcctcgcaccaagattacggttcctgcgcgttgggtggtgaatggtattgaacgcagcggcgaagttaatgccaaaccgggtaccaaaagcggtgaccgtgtgggcatctgggtcgatagcgccggtcagctggtcgacgagccggcaccgccagcgcgtgcgatcgccgatgcggcgctggctgccctgggtctgtggctgagcgtggcagcggtcgccggtgcgttgctggcgctgacgcgcgcaattctgatccgcgttcgcaatgcgagctggcagcacgatattgatagcctgttttgcacccaacgtgagctcatgaccacggcgcgtgatatcatgaatgcgggtgtcacctgtgttggcgagcacgaaacgttgaccgcagcagcacagtacatgcgcgaacatgatatcggcgcattgccgatttgcggcgacgatgatcgtctgcacggtatgctgaccgaccgcgatatcgttatcaagggtctggccgcaggcttggacccgaacaccgcgaccgccggtgaactggcacgtgacagcatctattacgtcgacgcgaacgccagcattcaagagatgctgaacgtgatggaagagcatcaggtgcgtcgtgtcccggttatcagcgaacatcgtctggttggtatcgttaccgaagccgacatcgcacgtcacctgccggagcacgcgattgttcagttcgtgaaagcgatttgcagcccgatggcgttggcgtctcgtcaaaagggcgacacaaaatttattctaaatgcaaagcttgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctga

SEQ ID NO: 74 - protein, 1091 aa, synthetic construct
  REGION 1..1091: note = Description of Artificial Sequence: PPE51-Rv1733c-Rv2626c-RpfB
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGEFMIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASRQKGDTKFILNAKLACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 75 - DNA, 31 bp, synthetic construct
  misc_feature 1..31: note = Description of Artificial Sequence: 85B For NdeI primer
  source 1..31: mol_type = other DNA
atagatcatatgtttagccgtcctggcctgc

SEQ ID NO: 76 - DNA, 31 bp, synthetic construct
  misc_feature 1..31: note = Description of Artificial Sequence: 85B Rev EcoRI nostop primer
  source 1..31: mol_type = other DNA
ttaagagaattcgcccgcacccagagaggat

SEQ ID NO: 77 - DNA, 32 bp, synthetic construct
  misc_feature 1..32: note = Description of Artificial Sequence: ESAT-6 For BamHI primer
  source 1..32: mol_type = other DNA
aacgttggatccatgacagagcagcagtggaa

SEQ ID NO: 78 - DNA, 32 bp, synthetic construct
  misc_feature 1..32: note = Description of Artificial Sequence: ESAT-6 Rev EcoRI ns primer
  source 1..32: mol_type = other DNA
atactagaattctgcgaacatcccagtgacgt

SEQ ID NO: 79 - DNA, 33 bp, synthetic construct
  misc_feature 1..33: note = Description of Artificial Sequence: 1733 For EcoRI primer
  source 1..33: mol_type = other DNA
aacttagaattcatgattgcgactacccgtgat

SEQ ID NO: 80 - DNA, 30 bp, synthetic construct
  misc_feature 1..30: note = Description of Artificial Sequence: 1733 In1 Rev Xma primer
  source 1..30: mol_type = other DNA
gatatacccgggggcctccagacggtcggt

SEQ ID NO: 81 - DNA, 31 bp, synthetic construct
  misc_feature 1..31: note = Description of Artificial Sequence: 1733 Out For Xma primer
  source 1..31: mol_type = other DNA
aacgaacccggggtccaagacagccgtagcc

SEQ ID NO: 82 - DNA, 31 bp, synthetic construct
  misc_feature 1..31: note = Description of Artificial Sequence: 1733 Out Rev Xba primer
  source 1..31: mol_type = other DNA
taagtatctagaatcggcgatcgcacgcgct

SEQ ID NO: 83 - DNA, 32 bp, synthetic construct
  misc_feature 1..32: note = Description of Artificial Sequence: 1733 In2 For Xba primer
  source 1..32: mol_type = other DNA
atagaatctagacgcgcaattctgatccgcgt

SEQ ID NO: 84 - DNA, 32 bp, synthetic construct
  misc_feature 1..32: note = Description of Artificial Sequence: 1733 Rev ns SacI primer
  source 1..32: mol_type = other DNA
agataagagctcacgttgggtgcaaaacaggc

SEQ ID NO: 85 - DNA, 31 bp, synthetic construct
  misc_feature 1..31: note = Description of Artificial Sequence: 2626 For SacI primer
  source 1..31: mol_type = other DNA
atagaagagctcatgaccacggcgcgtgata

SEQ ID NO: 86 - DNA, 36 bp, synthetic construct
  misc_feature 1..36: note = Description of Artificial Sequence: 2626 Rev HindIII ns primer
  source 1..36: mol_type = other DNA
taaagaaagctttgcatttagaataaattttgtgtc

SEQ ID NO: 87 - DNA, 30 bp, synthetic construct
  misc_feature 1..30: note = Description of Artificial Sequence: RpfD For HindIII primer
  source 1..30: mol_type = other DNA
taactaaagcttttgctgggcctgagcacc

SEQ ID NO: 88 - DNA, 33 bp, synthetic construct
  misc_feature 1..33: note = Description of Artificial Sequence: RpfD Rev XhoI stop primer
  source 1..33: mol_type = other DNA
atctaactcgagctagtcatcgcggctaccgct

SEQ ID NO: 89 - DNA, 33 bp, synthetic construct
  misc_feature 1..33: note = Description of Artificial Sequence: ESAT6 For NdeI primer
  source 1..33: mol_type = other DNA
taagatcatatgacagagcagcagtggaatttc

SEQ ID NO: 90 - DNA, 32 bp, synthetic construct; identical to SEQ ID NO: 78
  misc_feature 1..32: note = Description of Artificial Sequence: ESAT-6 Rev EcoRI ns primer
  source 1..32: mol_type = other DNA

SEQ ID NO: 91 - DNA, 1263 bp, synthetic construct
  misc_feature 1..1263: note = Description of Artificial Sequence: Ag85B-ESAT6 fusion protein with Ag85B signal sequence
  source 1..1263: mol_type = other DNA
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggcatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatga

SEQ ID NO: 92 - protein, 420 aa, synthetic construct
  REGION 1..420: note = Description of Artificial Sequence: Ag85B-ESAT6 (Ag85B signal sequence)
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 93 - DNA, 1272 bp, synthetic construct
  misc_feature 1..1272: note = Description of Artificial Sequence: Ag85B-ESAT6 fusion protein with 19 kDa lipoprotein signal sequence
  source 1..1272: mol_type = other DNA
atgaagcgtggactgacggtcgcggtagccggagccgccattctggtcgcaggtctttccggatgttcaagcaacaagtcgactacaggaagcggtgagaccacgaccgcggcaggtaccacggcaagccccggccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggcatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatga

SEQ ID NO: 94 - protein, 423 aa, synthetic construct
  REGION 1..423: note = Description of Artificial Sequence: Ag85B-ESAT6 (19 kDa lipoprotein signal sequence)
MKRGLTVAVAGAAILVAGLSGCSSNKSTTGSGETTTAAGTTASPGRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 95 - DNA, 1020 bp, synthetic construct
  misc_feature 1..1020: note = Description of Artificial Sequence: Rv2626c-RpfD fusion protein with Ag85B signal sequence
  source 1..1020: mol_type = other DNA
atgacagacgtgagccgaaagattcgagcttggggacgccgattgatgatcggcacggcagcggctgtagtccttccgggcctggtggggcttgccggcggagcggcaaccgcgggcgcgttctccatgaccaccgcacgcgacatcatgaacgcaggtgtgacctgtgttggcgaacacgagacgctaaccgctgccgctcaatacatgcgtgagcacgacatcggcgcgttgccgatctgcggggacgacgaccggctgcacggcatgctcaccgaccgcgacattgtgatcaaaggcctggctgcgggcctagacccgaataccgccacggctggcgagttggcccgggacagcatctactacgtcgatgcgaacgcaagcatccaggagatgctcaacgtcatggaagaacatcaggtccgccgtgttccggtcatctcagagcaccgcttggtcggaatcgtcaccgaagccgacatcgcccgacacctgcccgagcacgccattgtgcagttcgtcaaggcaatctgctcgcccatggccctcgccagcatgacaccgggtttgcttactactgcgggtgctggccgaccacgtgacaggtgcgccaggatcgtatgcacggtgttcatcgaaaccgccgttgtcgcgaccatgtttgtcgcgttgttgggtctgtccaccatcagctcgaaagccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgattag

SEQ ID NO: 96 - protein, 339 aa, synthetic construct
  REGION 1..339: note = Description of Artificial Sequence: Rv2626c-RpfD (Ag85B signal sequence)
MTDVSRKIRAWGRRLMIGTAAAVVLPGLVGLAGGAATAGAFSMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASMTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 97 - DNA, 1029 bp, synthetic construct
  misc_feature 1..1029: note = Description of Artificial Sequence: Rv2626c-RpfD fusion protein with 19 kDa lipoprotein signal sequence
  source 1..1029: mol_type = other DNA
atgaagcgtggactgacggtcgcggtagccggagccgccattctggtcgcaggtctttccggatgttcaagcaacaagtcgactacaggaagcggtgagaccacgaccgcggcaggtaccacggcaagccccggcatgaccaccgcacgcgacatcatgaacgcaggtgtgacctgtgttggcgaacacgagacgctaaccgctgccgctcaatacatgcgtgagcacgacatcggcgcgttgccgatctgcggggacgacgaccggctgcacggcatgctcaccgaccgcgacattgtgatcaaaggcctggctgcgggcctagacccgaataccgccacggctggcgagttggcccgggacagcatctactacgtcgatgcgaacgcaagcatccaggagatgctcaacgtcatggaagaacatcaggtccgccgtgttccggtcatctcagagcaccgcttggtcggaatcgtcaccgaagccgacatcgcccgacacctgcccgagcacgccattgtgcagttcgtcaaggcaatctgctcgcccatggccctcgccagcatgacaccgggtttgcttactactgcgggtgctggccgaccacgtgacaggtgcgccaggatcgtatgcacggtgttcatcgaaaccgccgttgtcgcgaccatgtttgtcgcgttgttgggtctgtccaccatcagctcgaaagccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgattag

SEQ ID NO: 98 - protein, 342 aa, synthetic construct
  REGION 1..342: note = Description of Artificial Sequence: Rv2626c-RpfD (19 kDa lipoprotein signal sequence)
MKRGLTVAVAGAAILVAGLSGCSSNKSTTGSGETTTAAGTTASPGMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASMTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 99 - protein, 1062 aa, synthetic construct
  REGION 1..1062: note = Description of Artificial Sequence: Ag85B-ESAT6-Rv1733c-Rv2626c- RpfB (E. coli optimized)
MFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGGSMTEQQWNFAGIEAAASAIQGNVTSIHSLLDEGKQSLTKLAAAWGGSGSEAYQGVQQKWDATATELNNALQNLARTISEAGQAMASTEGNVTGMFAEFMIATTRDREGATMITFRLRLPCRTILRVFSRNPLVRGTDRLEAPGVQDSRSHVYAHQAQTRHPATATVIDHEGVIDSNTTATSAPPRTKITVPARWVVNGIERSGEVNAKPGTKSGDRVGIWVDSAGQLVDEPAPPARAIADSRRAILIRVRNASWQHDIDSLFCTQRELMTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASKLMLRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 100 - protein, 1034 aa, synthetic construct
  REGION 1..1034: note = Description of Artificial Sequence: Ag85B-ESAT6-Rv1733c-Rv2626c- RpfB (human optimized)
MFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGPGTEQQWNFAGIEAAASAIQGNVTSIHSLLDEGKQSLTKLAAAWGGSGSEAYQGVQQKWDATATELNNALQNLARTISEAGQAMASTEGNVTGMFAFEIATTRDREGATMITFRLRLPCRTILRVFSRNPLVRGTDRLEAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADRAILIRVRNASWQHDIDSLFCTQRRSTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASGAPACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

(19) United States
(12) Patent Application Publication
(10) Pub. No.: US 2024/0101612 A1
(43) Pub. Date: Mar. 28, 2024

(54) Tuberculosis Compositions And Methods Of Using The Same

(71) Applicant: International AIDS Vaccine Initiative, Inc., New York, NY (US)

(72) Inventors: Ravi Anantha, New York, NY (US); Nathalie Cadieux, New York, NY (US); Thomas G. Evans, New York, NY (US); Michele Stone, New York, NY (US); Barry Walker, New York, NY (US)

(21) Appl. No.: 18/461,754

(22) Filed: Sep. 6, 2023

Related U.S. Application Data
(63) Continuation of application No. 17/236,409, filed on Apr. 21, 2021, now Pat. No. 11,787,842.
Application No. 17/236,409 is a continuation of application No. 16/289,919, filed on Mar. 1, 2019, now Pat. No. 11,014,969.
Application No. 16/289,919 is a continuation of application No. 14/313,694, filed on Jun. 24, 2014, now Pat. No. 10,266,574.
(60) Provisional application No. 61/838,872, filed on Jun. 25, 2013.

(51) Int. Cl. C07K 14/35; A61K 39/04

(52) U.S. Cl. CPC C07K 14/35; A61K 39/04; A61K 2039/55561; C07K 2319/00

(57) ABSTRACT

The present disclosure provides fusion proteins comprising Mycobacterium tuberculosis (Mtb) antigens, nucleic acid molecules encoding the same, vectors comprising nucleic acid molecules, compositions comprising the same, and methods of eliciting an immune response against tuberculosis.

### REFERENCE TO SEQUENCE LISTING

This application includes a Sequence Listing filed electronically as an XML file named 901513171SEQ, created on Aug. 30, 2023, with a size of 823,687 bytes. The Sequence Listing is incorporated herein by reference.

### FIELD

The present disclosure is directed, in part, to fusion proteins comprising Mycobacterium tuberculosis (Mtb) antigens, nucleic acid molecules encoding the same, vectors comprising nucleic acid molecules, compositions comprising the same, and methods of eliciting an immune response against tuberculosis.

### BACKGROUND

Tuberculosis (TB) is a global health problem resulting in 8 million new cases and 2 million deaths each year. The emergence of multi-drug and totally-drug resistant strains of TB only makes this problem more severe. The life cycle of Mtb has 3 stages. In the acute phase following initial infection the bacteria replicate in the host and virulence factors are expressed, leading to the generation of an immune response by the host. As the immune response begins to control the infection, the Mtb enters a latent, asymptomatic state in which the bacteria become non-replicating and are encased in granulomas. The bacterium can persist in this latent state in infected individuals for many years, making diagnosis and treatment of disease difficult. In some cases, the bacteria are reactivated and begin replicating again, leading back to the disease state. Reactivation can occur for numerous reasons, including immune suppression caused by diseases such as HIV, treatments such as chemotherapy, or the weakening of the immune system due to aging. An estimated 2 billion people are latently infected with Mtb worldwide, and reactivation of latent Mtb accounts for most new cases of active TB disease. Reactivation is associated with inflammation, necrosis and cavitation of the lung, a process that results in draining of the lesions into the bronchus. Aerosols generated when individuals with bronchial lesions cough causes dissemination of the Mtb organism to uninfected, susceptible persons, and the transmission cycle is thus maintained.

The only currently available vaccine against TB, Mycobacterium bovis (Bacille Calmette-Guérin) (BCG), was first introduced in 1921. BCG has been widely utilized and while studies show that for some purposes BCG is effective (e.g. against disseminated TB), it is known to be ineffective with respect to preventing the development, persistence and reactivation of latent TB. There is an ongoing need to develop improved, more effective vaccines against TB. In particular, there is a need to develop vaccines that provide protection against the development, maintenance and/or reactivation of latent tuberculosis infection. With the availability of the entire genomic sequence of Mtb, and the tools for bioinformatic and experimental analysis of Mtb antigens, many new potential Mtb vaccine candidates have been identified in recent years. These include antigens that are involved in acute infection, maintenance of latency, or reactivation of Mtb. There are a range of delivery strategies in clinical development that are comprised of combinations of these and other antigens that have been tested in animal models and are currently or will soon be in clinical trials.

While vaccines are often effective to immunize individuals prophylactically or therapeutically against pathogen infection or human diseases, there is a need for improved vaccines. There is also a need for compositions and methods that produce an enhanced immune response. Likewise, while some immunotherapeutics are useful to modulate immune response in a patient, there remains a need for improved immunotherapeutic compositions and methods.

### SUMMARY

This disclosure describes an antigen cassette (and specified variants) that can be used to create tuberculosis vaccines comprising specified Mycobacterium tuberculosis (Mtb) antigens which are involved with 3 identified stages of disease: 1) infection or acute infection, 2) latency or the latent state, and 3) resuscitation or reactivation of active disease. The disclosure also describes the strategic combination of antigens which are incorporated into a variety of delivery platforms in such a way as to provide pathways to a matrix of matched combinations of antigen delivery to obtain an optimized immune response. The subject matter described herein can be used as a prophylactic or therapeutic TB vaccine. The initial selection of antigens for inclusion into a usable cassette was based on a number of parameters including, for example, a thorough review of the literature, expression data, responses by human T cells, inclusion of human immunogenic regions, mouse protection studies, and conservation in sequence across most strains of TB with full genome sequences. Specific antigens were then probed to be sure they were able to be expressed in a variety of systems (BCG, protein, viral vectors, nucleic acids), that they were immunogenic, and they could be made as fusions in proteins or other vectors to simplify downstream manufacturing concerns. All of the selected antigens were then shown to be immunogenic in mice, either when used alone, or in a variety of combinations, to arrive at the present application.

The constructs described herein have been integrated into a specified range of delivery platforms that include the following classes (but not exhaustive) of representative delivery platforms: 1) viral vector delivery systems, 2) recombinant BCG, 3) recombinant purified protein fusions. 4) DNA plasmid vector systems, and 5) RNA vector systems. These delivery platforms can be used either in a single platform alone or in combinations as matched antigen prime-boost approaches. In addition, the use of these antigens in a single rBCG vector system, which is envisioned to be used as an antigen matched prime for a boost with any of the modalities above, including protein, viral vectors, nucleic acids, or others.

The present disclosure provides fusion proteins that comprise at least three Mycobacterium tuberculosis (Mtb) antigens, wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides nucleic acid molecules encoding fusion proteins that comprise at least three Mycobacterium tuberculosis (Mtb) antigens, wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides: compositions comprising the fusion proteins and a pharmaceutically acceptable carrier; vectors encoding the fusion proteins; compositions comprising the vectors and a pharmaceutically acceptable carrier; cells comprising the vectors; compositions comprising the cells and a pharmaceutically acceptable carrier.

The present disclosure also provides compositions that comprise at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen.

The present disclosure also provides compositions that comprise at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; wherein the composition comprises at least one nucleic acid molecule encoding at least one of the Mtb antigens.

The present disclosure also provides methods of eliciting an immune response against Mycobacterium tuberculosis in a mammal comprising administering to the mammal an immunologically sufficient amount of one or more fusion proteins comprising at least three Mycobacterium tuberculosis (Mtb) antigens, wherein at least one fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides methods of eliciting an immune response against Mycobacterium tuberculosis in a mammal comprising administering to the mammal an immunologically sufficient amount of a composition comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides methods of eliciting an immune response against Mycobacterium tuberculosis in a mammal comprising administering to the mammal an immunologically sufficient amount of a composition comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides fusion proteins for use in the preparation of a medicament for treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides fusion proteins for use in treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides uses of a fusion protein in the preparation of a medicament for treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides uses of a fusion protein in treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides compositions for use in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides composition for use in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides uses of a composition in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides uses of a composition in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides compositions for use in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides compositions for use in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides uses of a composition in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides uses of a composition in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides fusion proteins, compositions, cells, vectors, methods, and uses, as described herein, substantially as described with reference to the accompanying examples and/or figures.

### BRIEF DESCRIPTION OF THE DRAWINGS

FIGS. 1A and 1B show maps of the plasmids used to insert the genes of interest into the chromosome of BCG SSI or other strains of BCG: (A) constructs with the Ag85B signal sequence for secretion of the fusions; and (B) constructs with the 19 kDa signal sequence to anchor the fusions into the membrane.

FIG. 2 shows in vitro antigen responsiveness after vaccination with BCG strains carrying antigen cassette.

FIGS. 3A and 3B show in vitro stimulation: (A) showing INF-γ induction in splenocytes following protein stimulation and ELISpot; and (B) analysis showing number of splenocytes expressing INF-γ from CB6F1 mice immunized twice with 10 μg of the 5 Ag fusion protein (Construct D) and a synthetic poly I:C adjuvant.

FIGS. 4A and 4B show in vitro stimulation: (A) ELISpot; and (B) analysis of splenocytes from CB6F1 mice immunized twice with 3 μg of the 5 Ag fusion protein (Construct D) or 4 Ag fusion protein (Construct A) and a synthetic MPL TLR4 adjuvant; when Ag85B is removed from the 5 Ag fusion protein, the responses to the other 4 antigens in the 4 Ag protein increase.

FIGS. 5A and 5B show in vitro stimulation: (A) ELISpot; and (B) analysis of splenocytes from CB6F1 mice immunized twice with 3 μg of the 5 Ag fusion protein (Construct D) and 4 Ag fusion proteins with either wild-type or modified Rv1733 or the 4 Ag protein with RpfD replaced by RpfB, with RpfB either at the 3′ or 5′ end of the fusion protein; the proteins were adjuvanted with a synthetic poly I:C adjuvant; RpfB induces a much stronger immune response than RpfD, particularly when RpfB is at the 5′ end of the fusion protein; neither modified nor wild-type Rv1733 induced a strong immune response.

FIGS. 6A and 6B show bacterial numbers in: (A) the lungs and (B) spleen of CB6F1 mice primed with BCG and boosted with either the 4 Ag or 5 Ag fusion protein 4 weeks after challenge with Mycobacterium tuberculosis H37Rv.

### DESCRIPTION OF EMBODIMENTS

The terminology used herein is for the purpose of describing particular embodiments only and is not intended to be limiting.

As used herein, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, “acute Mtb antigen” means any Mtb antigen involved in the acute phase tuberculosis infection.

As used herein, “adjuvant” means any molecule added to any composition described herein to enhance the immunogenicity of the Mtb antigens.

As used herein, “coding sequence” or “encoding nucleic acid” means the nucleic acids (RNA or DNA molecule) that comprise a nucleotide sequence which encodes an Mtb antigen. The coding sequence can further include initiation and termination signals operably linked to regulatory elements including a promoter and polyadenylation signal capable of directing expression in the cells of an individual or mammal to which the nucleic acid is administered.

As used herein, “consensus” or “consensus sequence” means a polypeptide sequence based on analysis of an alignment of multiple subtypes of a particular Mtb antigen. Nucleic acid sequences that encode a consensus polypeptide sequence can be prepared. Vaccines comprising Mtb antigens that comprise consensus sequences and/or nucleic acid molecules that encode such antigens can be used to induce broad immunity against multiple subtypes or serotypes of a particular antigen.

As used herein, “electroporation” means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

As used herein, “fragment” with respect to nucleic acid sequences, means a nucleic acid sequence or a portion thereof, that encodes a portion of an Mtb antigen capable of eliciting an immune response in a mammal that cross reacts with a full length wild type Mtb antigen. The fragments can be DNA fragments selected from at least one of the various nucleotide sequences that encode protein fragments set forth below.

As used herein, “fragment” or “immunogenic fragment” with respect to polypeptide sequences, means a portion of an MTB antigen capable of eliciting an immune response in a mammal that cross reacts with a full length wild type strain Mtb antigen. Fragments of consensus or wild type Mtb antigens can comprise at least 10%, at least 20%, at least 30%, at least 40%, at least 50%, at least 60%, at least 70%, at least 80%, at least 90% or at least 95% of a consensus or wild type Mtb antigen. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120) amino acids or more, at least 130 amino acids or more, at least 140 amino acids or more, at least 150 amino acids or more, at least 160 amino acids or more, at least 170 amino acids or more, at least 180 amino acids or more of a consensus or wild type protein.

As used herein, “genetic construct” refers to the DNA or RNA molecules that comprise a nucleotide sequence which encodes an Mtb antigen. The coding sequence includes initiation and termination signals operably linked to regulatory elements including a promoter and polyadenylation signal capable of directing expression in the cells of the individual to whom the nucleic acid molecule is administered.

As used herein, “expressible form” refers to gene constructs that contain the necessary regulatory elements operable linked to a coding sequence that encodes an Mtb antigen such that when present in the cell of the individual, the coding sequence will be expressed.

As used herein, “homology” refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous” refers to a probe that can hybridize to a strand of the double-stranded nucleic acid sequence under conditions of low stringency. When used in reference to a single-stranded nucleic acid sequence, the term “substantially homologous” refers to a probe that can hybridize to (i.e., is the complement of) the single-stranded nucleic acid template sequence under conditions of low stringency.

As used herein, “identical” or “identity” in the context of two or more nucleic acids or polypeptide sequences, means that the sequences have a specified percentage of residues that are the same over a specified region. The percentage can be calculated by optimally aligning the two sequences, comparing the two sequences over the specified region. determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) residues can be considered equivalent. Identity can be performed manually or by using a computer sequence algorithm such as BLAST or BLAST 2.0.

As used herein, “immune response” means the activation of a host's immune system, e.g., that of a mammal, in response to the introduction of an Mtb antigen. The immune response can be in the form of a cellular or humoral response, or both.

As used herein, “latent Mtb antigen” means any Mtb antigen involved in the latent phase tuberculosis infection.

As used herein, “Mtb antigen” means an antigen from Mycobacterium tuberculosis, which may be an isolated antigen, or an antigen that forms part of a fusion protein with other antigen(s).

As used herein, “nucleic acid” or “oligonucleotide” or “polynucleotide” means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “promoter” means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, “resuscitation Mtb antigen” means any Mtb antigen involved in the resuscitation or reactivation of a tuberculosis infection.

As used herein, “signal peptide” and “leader sequence”, used interchangeably, refer to an amino acid sequence that can be linked at the amino terminus of an Mtb antigenic protein set forth herein. Signal peptides/leader sequences typically direct localization of a protein. Signal peptides/leader sequences used herein can facilitate secretion of the protein from the cell in which it is produced or anchor it in the membrane. Signal peptides/leader sequences are often cleaved from the remainder of the protein, often referred to as the mature protein, upon secretion from the cell. Signal peptides/leader sequences are linked at the N terminus of the protein.

As used herein, “stringent hybridization conditions” means conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions can be selected to be about 5 to 10° C. lower than the thermal melting point (Tₘ) for the specific sequence at a defined ionic strength pH. The Tₘ can be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can be those in which the salt concentration is less than about 1.0 M sodium ion, such as about 0.01 to 1.0 M sodium ion concentration (or other salts) at pH 7.0 to 8.3 and the temperature is at least about 30° C. for short probes (e.g., about 10 to 50 nucleotides) and at least about 60° C. for long probes (e.g., greater than about 50 nucleotides). Stringent conditions can also be achieved with the addition of destabilizing agents such as formamide. For selective or specific hybridization, a positive signal can be at least 2 to 10 times background hybridization. Exemplary stringent hybridization conditions include the following: 50% formamide, 5× SSC, and 1% SDS, incubating at 42° C., or, 5× SSC, 1% SDS, incubating at 65° C., with wash in 0.2× SSC, and 0.1% SDS at 65° C.

As used herein, “substantially complementary” means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used herein, “substantially identical” means that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used herein, “variant” with respect to a nucleic acid means: i) a portion or fragment of a referenced nucleotide sequence; ii) the complement of a referenced nucleotide sequence or portion thereof; iii) a nucleic acid that is substantially identical to a referenced nucleic acid or the complement thereof; or iv) a nucleic acid that hybridizes under stringent conditions to the referenced nucleic acid, complement thereof, or a sequences substantially identical thereto.

As used herein, “variant” with respect to a peptide or polypeptide means that it differs in amino acid sequence by the insertion, deletion, or conservative substitution of amino acids, but retains at least one biological activity. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. Amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “vector” means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector.

The present disclosure provides fusion proteins comprising at least three Mycobacterium tuberculosis (Mtb) antigens. In some embodiments, the fusion protein comprises at least one acute Mtb antigen, at least one latent Mtb antigen, and at least one resuscitation Mtb antigen. In some embodiments, the fusion protein comprises at least two latent Mtb antigens and at least one resuscitation Mtb antigen.

In some embodiments, the nucleic acid molecule encoding any particular Mtb antigen can be a mycobacterial sequence, a bacterial codon optimized sequence (such as an E. coli optimized sequence), or a mammalian optimized sequence (such as a human optimized sequence). The E. coli optimized sequences can be used, for example, to produce fusion proteins. The human optimized sequences can be used in, for example, viral vectors. Methods of codon optimization (whether for bacterial or mammalian) are well known to the skilled artisan.

In some embodiments, the acute Mtb antigen is Ag85B, ESAT6, MPT64, PPE15, PPE51, or Rv3615c. In some embodiments, the acute Mtb antigen is Ag85B, ESAT6, or Rv3615c. In some embodiments, the acute Mtb antigen is Ag85B or ESAT6. Additional acute Mtb antigens are well known to the skilled artisan.

The acute Mtb antigen Ag85B is also known as Rv1886c. A nucleotide sequence encoding Ag85B is shown in Table 1 as SEQ ID NO:1 (mycobacterial sequence; not codon optimized), SEQ ID NO:2 (E. coli optimized), and SEQ ID NO:3 (human optimized), and an amino acid sequence of Ag85B is shown in Table 1 as SEQ ID NO:4 (mycobacterial sequence) and SEQ ID NO:5 (E. coli and human optimized).

The acute Mtb antigen ESAT-6 is also known as Rv3875. A nucleotide sequence encoding ESAT-6 is shown in Table 1 as SEQ ID NO:6 (mycobacterial sequence; not codon optimized) and SEQ ID NO:7 (human optimized), and an amino acid sequence of ESAT-6 is shown in Table 1 as SEQ ID NO:8.

The acute Mtb antigen MPT64 is also known as Rv1980c. A nucleotide sequence encoding the acute Mtb antigen MPT64 is shown in Table 1 as SEQ ID NO:9 (mycobacterial sequence; not codon optimized) and as SEQ ID NO: 10 (human optimized), and an amino acid sequence of MPT64 is shown in Table 1 as SEQ ID NO:11.

The acute Mtb antigen PPE15 is also known as Rv1039c. A nucleotide sequence encoding the acute Mtb antigen PPE15 is shown in Table 1 as SEQ ID NO: 12 (mycobacterial sequence; not codon optimized) and as SEQ ID NO: 13 (human optimized), and an amino acid sequence of PPE15 is shown in Table 1 as SEQ ID NO:14.

The acute Mtb antigen PPE51 is also known as Rv3136. A nucleotide sequence encoding the acute Mtb antigen PPE51 is shown in Table 1 as SEQ ID NO: 15 (mycobacterial sequence; not codon optimized), SEQ ID NO:16 (E. coli optimized) and as SEQ ID NO:17 (human optimized), and an amino acid sequence of PPE51 is shown in Table 1 as SEQ ID NO:18.

A nucleotide sequence encoding the acute Mtb antigen Rv3615c is shown in Table 1 as SEQ ID NO:19 (mycobacterial sequence; not codon optimized) and as SEQ ID NO:20 (human optimized), and an amino acid sequence of Rv3615c is shown in Table 1 as SEQ ID NO:21.

TABLE 1

| | nucleotide sequence |
|---|---|
| Construct | amino acid sequence |
| Ag85B | atgacagacgtgagccgaaagattcgagcttggggacgccgattgatgatcggcacggcagcggctgtagt |
| | ccttccgggcctggtggggcttgccgcggagcggcaaccgcgggcgcgttctcccggccggggctgccg |
| | gtcgagtacctgcaggtgccgtcgccgtcgatgggccgcgacatcaaggttcagttccagagcggtgggaa |
| | caactcacctgcggtttatctgctcgacggcctgcgcgcccaagacgactacaacggctgggatatcaacac |
| | cccggcgttcgagtggtactaccagtcgggactgtcgatagtcatgccggtcggcgggcagtccagcttctac |
| | agcgactggtacagcccggcctgcggtaaggctggctgccagacttacaagtgggaaaccttcctgaccag |
| | cgagctgccgcaatggttgtccgccaacagggccgtgaagcccaccggcagcgctgcaatcggcttgtcga |
| | tggccggctcgtcggcaatgatcttggccgcctaccacccccagcagttcatctacgccggctcgctgtcggc |
| | cctgctggacccctctcaggggatggggcctagcctgatcggcctcgcgatgggtgacgccggcggttaca |
| | aggccgcagacatgtggggtccctcgagtgacccggcatgggagcgcaacgaccctacgcagcagatccc |
| | caagctggtcgcaaacaacacccggctatgggtttattgcgggaacggcaccccgaacgagttgggcggtg |
| | ccaacatacccgccgagttcttggagaacttcgttcgtagcagcaacctgaagttccaggatgcgtacaacgc |
| | cgcgggcgggcacaacgccgtgttcaacttcccgcccaacggcacgcacagctgggagtactggggcgct |
| | cagctcaacgccatgaagggtgacctgcagagttcgttaggcgccggctga (SEQ ID NO: 1) |
| | atgtttagccgtcctggcctgccagttgaatacctgcaagttccgagcccgtccatgggtcgtgacattaaggt |
| | gcagttccagagcggcggtaacaatagcccggctgtgtacctgctggacggtctgcgtgcgcaggatgatta |
| | caacggctgggacatcaataccccggcatttgagtggtattaccagtcgggtctgagcattgtgatgccggttg |
| | gcggtcaaagcagcttctatagcgattggtacagcccggcatgcggcaaggctggttgccaaacctacaagt |
| | gggaaactttcttgaccagcgagctgccgcaatggttgagcgccaaccgtgcggtcaaaccgaccggtagc |
| | gctgctattggcctgtccatggccggcagcagcgcgatgatcttggcggcataccatccgcagcagtttatcta |
| | cgccggtagcctgagcgcattgctggacccgagccaaggcatgggtccgagcctgattggtctggcaatgg |
| | gtgacgcaggtggttacaaagcggccgatatgtggggcccatctagcgacccggcatgggagcgtaatgac |
| | ccgacccagcaaattccgaaactggtggcgaataacacgcgcctgtgggtctactgtggcaatggtacgccg |
| | aacgagctgggtggcgcgaatatccctgcggagtttctggaaaactttgttcgcagcagcaacctgaaattcca |
| | ggacgcgtataacgcagccggtggtcacaatgcggttttcaatttcccgccaaatggcactcatagctgggag |
| | tactggggtgcgcagttgaacgcaatgaaaggcgatctgcaatcctctctgggtgcgggc (SEQ ID |
| | NO: 2) |
| | atgttctccaggcccggcctgcctgtcgagtatctgcaggtcccctccccctccatgggcagagacatcaagg |
| | tgcagttccaatccggaggcaacaacagccccgccgtgtatctcctcgacggcctgagggctcaggacgact |
| | acaacggctgggacatcaacacccccgccttcgagtggtactaccagtccggactgagcatcgtcatgcccg |
| | tgggcggccagagctccttctacagcgactggtatagccctgcctgcggcaaagccggatgccagacctaca |
| | agtgggagacctttctgaccagcgaactgccccagtggctgtccgccaatagggccgtcaaacctaccggct |
| | ccgctgccatcggactcagcatggccggaagctccgctatgatcctggccgcctaccacccccagcaatttat |
| | ctacgctggcagcctgtccgctctgctggatcctagccaaggcatgggccctagcctcattggcctggccatg |
| | ggcgatgctggcggctataaggccgccgatatgtggggccctagctccgatcctgcctgggagaggaatga |
| | ccccacccagcagatccccaagctggtggccaacaacacaaggctctgggtgtactgcggcaatggcaccc |
| | ccaacgaactgggcggagccaacattcccgccgagttcctggagaacttcgtcaggagcagcaacctgaag |
| | ttccaggacgcctacaatgccgccggaggccacaacgctgtgttcaacttccctcccaacggcacccacagc |
| | tgggagtattggggcgctcagctgaacgccatgaaaggcgacctccagagctccctgggagctgga |
| | (SEQ ID NO: 3) |
| | MTDVSRKIRAWGRRLMIGTAAAVVLPGLVGLAGGAATAGAFSRPGL |
| | PVEYLQVPSPSMGRDIKVQFQSGGNNSPAVYLLDGLRAQDDYNGWD |
| | INTPAFEWYYQSGLSIVMPVGGQSSFYSDWYSPACGKAGCQTYKWE |
| | TFLTSELPQWLSANRAVKPTGSAAIGLSMAGSSAMILAAYHPQQFIY |
| | AGSLSALLDPSQGMGPSLIGLAMGDAGGYKAADMWGPSSDPAWER |
| | NDPTQQIPKLVANNTRLWVYCGNGTPNELGGANIPAEFLENFVRSSN |
| | LKFQDAYNAAGGHNAVFNFPPNGTHSWEYWGAQLNAMKGDLQSSL |
| | GAG (SEQ ID NO: 4) |
| | MFSRPGLPVEYLQVPSPSMGRDIKVQFQSGGNNSPAVYLLDGLRAQD |
| | DYNGWDINTPAFEWYYQSGLSIVMPVGGQSSFYSDWYSPACGKAGC |
| | QTYKWETFLTSELPQWLSANRAVKPTGSAAIGLSMAGSSAMILAAY |
| | HPQQFIYAGSLSALLDPSQGMGPSLIGLAMGDAGGYKAADMWGPSS |
| | DPAWERNDPTQQIPKLVANNTRLWVYCGNGTPNELGGANIPAEFLE |
| | NFVRSSNLKFQDAYNAAGGHNAVFNFPPNGTHSWEYWGAQLNAMK |
| | GDLQSSLGAG (SEQ ID NO: 5) |
| ESAT6 | atgacagagcagcagtggaatttcgcgggtatcgaggccgcggcaagcgcaatccagggaaatgtcacgtc |
| | cattcattccctccttgacgaggggaagcagtccctgaccaagctcgcagcggcctggggcggtagcggttc |
| | ggaggcgtaccagggtgtccagcaaaaatgggacgccacggctaccgagctgaacaacgcgctgcagaa |
| | cctggcgcggacgatcagcgaagccggtcaggcaatggcttcgaccgaaggcaacgtcactgggatgttcg |
| | catag (SEQ ID NO: 6) |
| | accgagcagcagtggaacttcgccggcatcgaagctgccgctagcgccatccaaggcaacgtgaccagcat |
| | ccacagcctgctggacgagggcaagcagagcctgaccaagctggctgctgcttggggcggatccggaagc |
| | gaagcctaccagggcgtgcagcagaagtgggacgccacagccaccgagctgaacaacgccctgcagaac |
| | ctcgccagaaccatcagcgaggccggacaggctatggccagcacagagggcaatgtgaccggcatgttcg |
| | cc (SEQ ID NO: 7) |
| | TEQQWNFAGIEAAASAIQGNVTSIHSLLDEGKQSLTKLAAAWGGSGS |
| | EAYQGVQQKWDATATELNNALQNLARTISEAGQAMASTEGNVTGM |
| | FA (SEQ ID NO: 8) |
| MPT64 | gtgcgcatcaagatcttcatgctggtcacggctgtcgttttgctctgttgttcgggtgtggccacggccgcgccc |
| | aagacctactgcgaggagttgaaaggcaccgataccggccaggcgtgccagattcaaatgtccgacccggc |
| | ctacaacatcaacatcagcctgcccagttactaccccgaccagaagtcgctggaaaattacatcgcccagacg |
| | cgcgacaagttcctcagcgcggccacatcgtccactccacgcgaagccccctacgaattgaatatcacctcg |
| | gccacataccagtccgcgataccgccgcgtggtacgcaggccgtggtgctcaaggtctaccagaacgccgg |
| | cggcacgcacccaacgaccacgtacaaggccttcgattgggaccaggcctatcgcaagccaatcacctatg |
| | acacgctgtggcaggctgacaccgatccgctgccagtcgtcttccccattgtgcaaggtgaactgagcaagc |
| | agaccggacaacaggtatcgatagcgccgaatgccggcttggacccggtgaattatcagaacttcgcagtca |
| | cgaacgacggggtgattttcttcttcaacccgggggagttgctgcccgaagcagccggcccaacccaggtat |
| | tggtcccacgttccgcgatcgactcgatgctggcctag (SEQ ID NO: 9) |
| | atggtcaggatcaagatcttcatgctcgtgaccgccgtggtgctcctgtgttgttccggcgtggctaccgctgct |
| | cccaagacctactgcgaggagctgaagggaaccgacaccggccaggcctgccagatccaaatgagcgac |
| | cccgcctacaacatcaacatctccctcccctcctactaccccgatcagaagtccctcgagaactacatcgctca |
| | gaccagggacaagttcctgagcgccgccacaagcagcacacccagagaggccccctacgagctgaacatc |
| | acctccgccacctaccagtccgctattcctcccagaggcacccaggctgtggtgctcaaggtctaccaaaacg |
| | ctggcggaacacaccccaccaccacctacaaggccttcgactgggaccaggcctacaggaagcccatcac |
| | atacgacaccctgtggcaggctgataccgatcccctgcccgtggtgttccccatcgtgcagggcgagctctcc |
| | aagcagaccggccagcaagtgagcatcgcccccaatgctggactggaccccgtgaactaccagaacttcgc |
| | cgtcaccaacgacggcgtgatcttcttcttcaatcccggcgaactgctgcctgaagctgctggccccacccaa |
| | gtgctggtgcctagaagcgccatcgactccatgctggcctga (SEQ ID NO: 10) |
| | VRIKIFMLVTAVVLLCCSGVATAAPKTYCEELKGTDTGQACQIQMSD |
| | PAYNINISLPSYYPDQKSLENYIAQTRDKFLSAATSSTPREAPYELNITS |
| | ATYQSAIPPRGTQAVVLKVYQNAGGTHPTTTYKAFDWDQAYRKPIT |
| | YDTLWQADTDPLPVVFPIVQGELSKQTGQQVSIAPNAGLDPVNYQNF |
| | AVTNDGVIFFFNPGELLPEAAGPTQVLVPRSAIDSMLA (SEQ ID NO: 11) |
| PPE15 | atggatttcggagctttaccccctgagatcaactccgcacgcatgtacgccggcgcgggtgcaggaccgatg |
| | atggccgccggggccgcatggaacggcctggccgccgagttgggtacgacggccgcgtcgtatgagtcgg |
| | tgatcacccggctgaccaccgagtcgtggatgggtccggcctcgatggcgatggtcgccgcagcccagccc |
| | tatctggcttggttgacctacaccgccgaagccgctgcgcatgccggctcgcaggccatggcgtcggcggc |
| | cgcctacgaggcggcctatgcgatgacagtgccgccggaggtggtcgcggccaaccgggcgctgctggc |
| | ggccctggtcgcgacgaacgtcctggggatcaacacaccggcaatcatggcgaccgaagccctctatgccg |
| | agatgtgggctcaggacgctctggctatgtacggctacgcggccgcttcgggagccgccgggatgctgcaa |
| | ccgttaagcccgccgtcgcagaccaccaacccgggcgggctggccgcccagtccgccgcggtcggctcg |
| | gctgccgccaccgccgccgtcaaccaggtgagcgtagcggacctgatcagtagcctgcccaacgcggtga |
| | gtgggctcgcctccccagtcacatcggttctcgactcgacggggctgagcggaatcattgccgacatcgacg |
| | ccctgctcgcgaccccgttcgtggcaaacatcatcaacagcgcagtcaacaccgccgcttggtatgtcaacgc |
| | cgccatccccaccgcgatattcctagcaaatgccctgaacagtggggcgccggtagcgatcgccgaaggcg |
| | ccatcgaggctgccgagggtgccgccagtgcggccgccgcggggttggcggactcggtgacgccagcgg |
| | ggctcggcgcaagtttaggcgaggccaccctggtcggccggctgtcagtgccggcggcctggtctacggcc |
| | gcaccggcgacaaccgccggcgccacagcgctcgaaggcagcggctggaccgtcgccgccgaagaagc |
| | cggcccagttaccgggatgatgccgggaatggcctcggccgccaagggcaccggtgcctatgccgggccg |
| | cggtacggattcaagcccactgtcatgcccaaacaggtcgtcgtgtga (SEQ ID NO: 12) |
| | atggattttggcgccctgcctcccgagatcaacagcgctaggatgtatgctggcgctggagccggacctatga |
| | tggccgctggagccgcctggaatggactggctgccgaactgggcacaacagccgcttcctacgagtccgtg |
| | atcaccagactcaccacagagtcctggatgggacctgccagcatggctatggtcgccgctgctcaaccctac |
| | ctggcctggctgacctatacagctgaagccgctgctcacgccggaagccaagctatggctagcgccgccgct |
| | tatgaggccgcttatgccatgaccgtgcctcccgaggtcgtggctgccaacagagctctcctggccgccctcg |
| | tggctaccaacgtgctgggaatcaacacccccgctattatggccaccgaggctctgtacgctgagatgtgggc |
| | ccaggatgccctcgccatgtacggatacgccgctgcttccggagctgctggaatgctgcagcccctgtcccc |
| | cccttcccagaccaccaaccccggaggactggctgctcaaagcgctgctgtgggatccgctgctgctaccgc |
| | tgccgtcaatcaggtcagcgtcgccgacctcatctccagcctgcctaacgctgtgagcggactggcctcccct |
| | gtcacatccgtgctcgatagcaccggcctgtccggcatcatcgccgacattgatgctctcctcgccacccccttt |
| | gtcgccaacatcatcaattccgccgtgaacaccgctgcctggtacgtcaacgctgccattcccaccgccatctt |
| | cctcgccaacgccctgaactccggagctcctgtcgccatcgctgagggcgctattgaggctgctgaaggagc |
| | cgctagcgctgctgctgctggactggctgatagcgtcacccctgctggactcggagctagcctgggagaagc |
| | caccctggtcggcagactgtccgtgcctgctgcttggagcaccgctgctcctgctacaaccgctggagctacc |
| | gctctggagggatccggatggacagtggctgctgaggaagctggacccgtgaccggaatgatgcctggcat |
| | ggccagcgctgctaagggaaccggcgcctatgccggacccagatacggattcaagcccaccgtcatgccca |
| | agcaggtcgtcgtctaa (SEQ ID NO: 13) |
| | MDFGALPPEINSARMYAGAGAGPMMAAGAAWNGLAAELGTTAAS |
| | YESVITRLTTESWMGPASMAMVAAAQPYLAWLTYTAEAAAHAGSQ |
| | AMASAAAYEAAYAMTVPPEVVAANRALLAALVATNVLGINTPAIM |
| | ATEALYAEMWAQDALAMYGYAAASGAAGMLQPLSPPSQTTNPGGL |
| | AAQSAAVGSAAATAAVNQVSVADLISSLPNAVSGLASPVTSVLDSTG |
| | LSGIIADIDALLATPFVANIINSAVNTAAWYVNAAIPTAIFLANALNSG |
| | APVAIAEGAIEAAEGAASAAAAGLADSVTPAGLGASLGEATLVGRLS |
| | VPAAWSTAAPATTAGATALEGSGWTVAAEEAGPVTGMMPGMASA |
| | AKGTGAYAGPRYGFKPTVMPKQVVV (SEQ ID NO: 14) |
| PPE51 | atggatttcgcactgttaccaccggaagtcaactccgcccggatgtacaccggccctggggcaggatcgctgt |
| | tggctgccgcgggcggctgggattcgctggccgccgagttggccaccacagccgaggcatatggatcggt |
| | gctgtccggactggccgccttgcattggcgtggaccggcagcggaatcgatggcggtgacggccgctccct |
| | atatcggttggctgtacacgaccgccgaaaagacacagcaaacagcgatccaagccagggcggcagcgct |
| | ggccttcgagcaagcatacgcaatgaccctgccgccaccggtggtagcggccaaccggatacagctgctag |
| | cactgatcgcgacgaacttcttcggccagaacactgcggcgatcgcggccaccgaggcacagtacgccga |
| | gatgtgggcccaggacgccgccgcgatgtacggttacgccaccgcctcagcggctgcggccctgctgaca |
| | ccgttctccccgccgcggcagaccaccaacccggccggcctgaccgctcaggccgccgcggtcagccag |
| | gccaccgacccactgtcgctgctgattgagacggtgacccaagcgctgcaagcgctgacgattccgagcttc |
| | atccctgaggacttcaccttccttgacgccatattcgctggatatgccacggtaggtgtgacgcaggatgtcga |
| | gtcctttgttgccgggaccatcggggccgagagcaacctaggccttttgaacgtcggcgacgagaatccc |
| | gcggaggtgacaccgggcgactttgggatcggcgagttggtttccgcgaccagtcccggcggtggggtgtc |
| | tgcgtcgggtgccggcggtgcggcgagcgtcggcaacacggtgctcgcgagtgtcggccgggcaaactc |
| | gattgggcaactatcggtcccaccgagctgggccgcgccctcgacgcgccctgtctcggcattgtcgcccgc |
| | cggcctgaccacactcccggggaccgacgtggccgagcacgggatgccaggtgtaccgggggtgccagt |
| | ggcagcagggcgagcctccggcgtcctacctcgatacggggttcggctcacggtgatggcccacccaccc |
| | gcggcagggtaa (SEQ ID NO: 15) |
| | atggattttgcgctgctgccgccggaagtgaacagcgcgcgcatgtataccggcccgggcgcgggcagcct |
| | gctggcggcggcgggcggctgggatagcctggcggcggaactggcgaccaccgcggaagcgtatggca |
| | gcgtgctgagcggcctggcggcgctgcattggcgcggcccggcggcggaaagcatggcggtgaccgcg |
| | gcgccgtatattggctggctgtataccaccgcggaaaaaacccagcagaccgcgattcaggcgcgcgcggc |
| | ggcgctggcgtttgaacaggcgtatgcgatgaccctgccgccgccggtggtggcggcgaaccgcattcagc |
| | tgctggcgctgattgcgaccaacttttttggccagaacaccgcggcgattgcggcgaccgaagcgcagtatg |
| | cggaaatgtgggcgcaggatgcggcggcgatgtatggctatgcgaccgcgagcgcggcggcggcgctgc |
| | tgaccccgtttagcccgccgcgccagaccaccaacccggcgggcctgaccgcgcaggcggcggcggtga |
| | gccaggcgaccgatccgctgagcctgctgattgaaaccgtgacccaggcgctgcaggcgctgaccattccg |
| | agctttattccggaagattttacctttctggatgcgatttttgcgggctatgcgaccgtgggcgtgacccaggatg |
| | tggaaagctttgtggcgggcaccattggcgcggaaagcaacctgggcctgctgaacgtgggcgatgaaaac |
| | ccggcggaagtgaccccgggcgattttggcattggcgaactggtgagcgcgaccagcccgggcggcggc |
| | gtgagcgcgagcggcgcgggcggcgcggcgagcgtgggcaacaccgtgctggcgagcgtgggccgcg |
| | cgaacagcattggccagctgagcgtgccgccgagctgggcggcgccgagcacccgcccggtgagcgcgc |
| | tgagcccggcgggcctgaccaccctgccgggcaccgatgtggcggaacatggcatgccgggcgtgccgg |
| | gcgtgccggtggcggcgggccgcgcgagcggcgtgctgccgcgctatggcgtgcgcctgaccgtgatgg |
| | cgcatccgccggcggcgggcgaattt (SEQ ID NO: 16) |
| | atggatttcgctctgctcccccccgaggtgaatagcgctaggatgtacacaggacccggagctggaagcctc |
| | ctggctgctgctggaggatgggactccctggctgccgagctcgctacaaccgctgaggcttacggaagcgtg |
| | ctctccggcctggctgctctccattggagaggccctgctgccgagtccatggctgtcacagccgctccclacat |
| | tggatggctgtacaccaccgccgagaagacccagcaaaccgctattcaggccagagctgccgccctggcct |
| | tcgaacaggcctacgctatgacactccccccccctgtcgtggctgccaataggatccagctcctggccctcat |
| | cgccaccaacttcttcggccaaaacaccgctgccatcgctgccaccgaagcccagtacgccgaaatgtggg |
| | cccaggatgccgctgctatgtacggctatgccacagctagcgctgccgctgctctgctcacacccttcagccc |
| | ccccaggcaaacaaccaaccctgccggactgacagcccaagctgctgccgtcagccaagctaccgacccc |
| | ctgagcctcctgatcgaaaccgtgacacaggccctgcaggccctgaccattcccagctttatccccgaggact |
| | tcacctttctggacgctatcttcgctggctacgccaccgtgggcgtgacacaagacgtcgagtccttcgtcgcc |
| | ggcacaatcggagccgagtccaacctcggactcctcaacgtcggcgacgaaaatcccgccgaagtgacac |
| | ctggagacttcggcattggagaactcgtcagcgccacatcccctggcggaggagtgagcgcttccggagct |
| | ggaggagctgcttccgtgggcaataccgtgctggccagcgtgggaagggccaactccattggccagctcag |
| | cgtccccccttcctgggctgccccttccacaaggcctgtgtccgctctcagccctgctggactgaccacactcc |
| | ctggcacagacgtggctgagcatggcatgcccggagtgcctggagtccctgtggctgctggcagagcttcc |
| | ggagtcctccctaggtatggcgtgaggctgacagtgatggctcatccccccgctgccggataa (SEQ ID |
| | NO: 17) |
| | MDFALLPPEVNSARMYTGPGAGSLLAAAGGWDSLAAELATTAEAY |
| | GSVLSGLAALHWRGPAAESMAVTAAPYIGWLYTTAEKTQQTAIQAR |
| | AAALAFEQAYAMTLPPPVVAANRIQLLALIATNFFGQNTAAIAATEA |
| | QYAEMWAQDAAAMYGYATASAAAALLTPFSPPRQTTNPAGLTAQA |
| | AAVSQATDPLSLLIETVTQALQALTIPSFIPEDFTFLDAIFAGYATVGV |
| | TQDVESFVAGTIGAESNLGLLNVGDENPAEVTPGDFGIGELVSATSPG |
| | GGVSASGAGGAASVGNTVLASVGRANSIGQLSVPPSWAAPSTRPVSA |
| | LSPAGLTTLPGTDVAEHGMPGVPGVPVAAGRASGVLPRYGVRLTVM |
| | AHPPAAG (SEQ ID NO: 18) |
| Rv3615c | atgacggaaaacttgaccgtccagcccgagcgtctcggtgtactggcgtcgcaccatgacaacgcggcggt |
| | cgatgcctcctcgggcgtcgaagctgccgctggcctaggcgaatctgtggcgatcactcacggtccgtactg |
| | ctcacagttcaacgacacgttaaatgtgtacttgactgcccacaatgccctgggctcgtccttgcatacggccg |
| | gtgtcgatctcgccaaaagtcttcgaattgcggcgaagatatatagcgaggccgacgaagcgtggcgcaag |
| | gctatcgacgggttgtttacctga (SEQ ID NO: 19) |
| | atgaccgagaacctgaccgtgcagcctgagaggctgggagtgctggccagccaccacgacaacgctgccg |
| | tggacgcttccagcggagtggaggctgctgctggactgggagagagcgtggccatcacccacggacccta |
| | ctgcagccagttcaacgacaccctgaacgtgtacctgacagcccacaacgccctgggaagcagcctgcata |
| | cagccggcgtggacctggctaagtccctgaggatcgccgccaagatctacagcgaggccgacgaggcctg |
| | gaggaaagccatcgacggcctgttcacctaa (SEQ ID NO: 20) |
| | MTENLTVQPERLGVLASHHDNAAVDASSGVEAAAGLGESVAITHGP |
| | YCSQFNDTLNVYLTAHNALGSSLHTAGVDLAKSLRIAAKIYSEADEA |
| | WRKAIDGLFT (SEQ ID NO: 21) |

In some embodiments, the latent Mtb antigen is Rv1733c, Rv2626c, Rv3407, or Rv2628c. In some embodiments, the latent Mtb antigen is Rv1733c or Rv2626c. Additional latent Mtb antigens are well known to the skilled artisan.

A nucleotide sequence encoding the wild type latent Mtb antigen Rv1733c is shown in Table 2 as SEQ ID NO:22 (mycobacterial sequence; not codon optimized), SEQ ID NO:23 (E. coli optimized), and an amino acid sequence of wild type Rv1733c is shown in Table 2 as SEQ ID NO:24. These sequences include two transmembrane regions of Rv1733c. A nucleotide sequence encoding Rv1733c, whereby both transmembrane regions are deleted is shown in Table 2 as SEQ ID NO:25 (E. coli optimized) and SEQ ID NO:26 (human optimized), and corresponding amino acid sequences are shown in Table 2 as SEQ ID NO:27 (E. coli optimized) and SEQ ID NO:28 (human optimized) (Rv1733C_{ΔTM}). In some embodiments, only a portion of the first and/or second or both transmembrane regions are deleted. In the E. coli optimized nucleotide sequence (SEQ ID NO:23), an XmaI restriction site was added, corresponding to an addition of amino acids PG; and an XbaI restriction site was added, corresponding to an addition of amino acids SR (see underlined and bolded added sequences).

A nucleotide sequence encoding the latent Mtb antigen Rv2626c is shown in Table 2 as SEQ ID NO:29 (mycobacterial sequence; not codon optimized), SEQ ID NO:30 (E. coli optimized), and SEQ ID NO:31 (human optimized), and an amino acid sequence of Rv2626c is shown in Table 2 as SEQ ID NO:32.

A nucleotide sequence encoding the latent Mtb antigen Rv3407 is shown in Table 2 as SEQ ID NO:33 (mycobacterial sequence; not codon optimized), SEQ ID NO:34 (E. coli optimized), and SEQ ID NO:35 (human optimized), and an amino acid sequence of Rv3407 is shown in Table 2 as SEQ ID NO:36.

A nucleotide sequence encoding the latent Mtb antigen Rv2628c is shown in Table 2 as SEQ ID NO:37 (mycobacterial sequence; not codon optimized) and as SEQ ID NO:38 (human optimized), and an amino acid sequence of Rv2628c is shown in Table 2 as SEQ ID NO:39.

TABLE 2

| | nucleotide sequence |
|---|---|
| Construct | amino acid sequence |
| Rv1733c | atgatcgccacaacccgcgatcgtgaaggagccaccatgatcacgtttaggctgcgcttgccgtgccggac |
| | gatactgcgggtgttcagccgcaatccgctggtgcgtgggacggatcgactcgaggcggtcgtcatgctgc |
| | tggccgtcacggtctcgctgctgactatcccgttcgccgccgcggccggcaccgcagtccaggattcccgc |
| | agccacgtctatgcccaccaggcccagacccgccatcccgcaaccgcgaccgtgatcgatcacgagggg |
| | gtgatcgacagcaacacgaccgccacgtcagcgccgccgcgcacgaagatcaccgtgcctgcccgatgg |
| | gtcgtgaacggaatagaacgcagcggtgaggtcaacgcgaagccgggaaccaaatccggtgaccgcgtc |
| | ggcatagggtcgacagtgccggtcagctggtcgatgaaccagctccgccggcccgtgccattgcggatgc |
| | ggccctggccgccttgagactctagttgagcgtcgccgcggttgcgggcgccctgctggcgctcactcgg |
| | gcgattctgatccgcgttcgcaacgccagtEggcaacacgacatcgacagcctgttctgcacgcagcggtg |
| | a |
| | (SEQ ID NO: 22) |
| | atgattgcgactacccgtgatcgtgagggcacgaccatgatcacgttccatctgcgtctgccgtgtcgcacc |
| | attttgcgcgtgttttcgcgtaacccgctggtccgcggtaccgaccgtctggaggccgttgtcatgctgctgg |
| | cggttaccgtgagcctgctgacgatcccattcgcagcggcagctggcacggccgtccaagacagccgtag |
| | ccatgtgtatgctcaccaggctcaaacccgtcacccggctactgccactgttatcgatcacgaaggcgtgatt |
| | gactccaataccacggcaacctccgcaccgcctcgcaccaagattacggttcctgcgcgttgggtggtgaat |
| | ggtattgaacgcagcggcgaagttaatgccaaaccgggtaccaaaagcggtgaccgtgtgggcatctggg |
| | tcgataggccggtcagctggtcgacgagccggcaccgccagcgcgtgcgatcgccgatgcggcgctgg |
| | ctgccctgggtctgtggctgagcgtggcagcggtcgccggtgcgttgctggcgctgacgcgcgcaattctg |
| | atccgcgttcgcaatgcgagctggcagcacgatattgatagcctotttgcacccaacgt |
| | (SEQ ID NO: 23) |
| | MIATIRDREGATMITFRLRLPCRTILRVFSRNPLAIRGTDRLEAVVML |
| | LAVTAVSLLTIPFAAAAGTAVQDSRSHVYAHQAQTRHPATATVIDHE |
| | GVIDSNTTATSAPPRTKITVPARWVVNGIERSGEVNAKPGTKSGDRV |
| | GIWVDSAGQLVDEPAPPARAIADAALAALGLWLSVAAVAGALLAL |
| | TRAILIRVRNASWQHDIDSLFCTQR |
| | (SEQ ID NO: 24) |
| Rv1733c_{ΔTM} | atgattgcgactacccatgatcgtaagggcacgaccatgatcacgttccgtctgcgtctgccgtgtcgcacc |
| | attttgcgcgtgttttcgcgtaacccgctggtccgcggtaccgaccgtctggaggcccccggggtccaagac |
| | agccgtagccatgtgtatgctcaccaggctcaaacccgtcacccggctactgccactgttatcgatcacgaa |
| | ggcgtgattgactccaataccacggcaacctccgcaccgcctcgcaccaagattacggttcctgcgcgttgg |
| | gtggtgaatggtattgaacgcagcggcgaagttaatgccaaaccgggtaccaaaagcggtgaccgtgtgg |
| | gcatctgggtcgatagcgccggtcagctggtcgacgagccggcaccgccagcgcgtgcgatcgccgatt |
| | ctagacgcgcaattctgatccgcgttcgcaatgcgagctggcagcacgatattgatagcctgttttgcaccca |
| | acgt |
| | (SEQ ID NO: 25) |
| | atcgccaccaccagggacagggaaggcgctaccatgatcaccttcaggctgaggctcccctgcaggacca |
| | tcctgagggtgttcagcaggaaccccctggtgaggggcaccgacagactggaagccgtgcaggacagca |
| | ggagccacgtgtatacccaccaggctcagaccaggcaccctgctaccgccaccgtgatcgaccacgagg |
| | gcgtgatcgactccaacaccaccgccaccagcgctcctcccagaaccaagatcacagtgcccgccaggtg |
| | ggtggtgaacggcatcgagaggagcggcgaggtgaacgccaagcctggaaccaagagcggcgacagg |
| | gtgggcatttgggtcgatagcgccggccagctggtggatgaacctgctccccctgccagagccatcgccg |
| | atagggccatcctgatcaggatgaggaacgccagctggcagcacgacatcgacagcctgttctgcaccca |
| | aagg |
| | (SEQ ID NO: 26) |
| | MIATTRDREGATMITFRLRITCRTILRVFSRNPLVRGTDRLEAPGVQ |
| | DSRSHVYAHQAQTRHPATATVIDHEGVIDSNTTATSAPPRTKITVPA |
| | RWVVNGIERSGEVNAKPGTKSGDRVGIWVDSAGQLVDEPAPPARAI |
| | ADSRRAILIRVRNASWQHDIDSLFCTQR |
| | (SEQ ID NO: 27) |
| | IATTRDREGATMITFRLRLPCRTILRVFSRNPLVRGTDRLEAVQDSRS |
| | HVYAHQAQTRHPATATVIDHEGVIDSNTTATSAPPRTKITVPARWV |
| | VNGIERSGEVNAKPGTKSGDRVGIWVDSAGQLVDEPAPPARAIADR |
| | AILIRVRNASWQHDIDSLFCTQR |
| | (SEQ ID NO: 28) |
| Rv2626c | atgaccaccgcacgcgacatcatgaacgcaggtgtgacctgtgttggcgaacacgagacgctaaccgag |
| | ccgctcaatacatgcgtgagcacgacatcggcgcgagccgatctgcggggacgacgaccggctgcacg |
| | gcatgctcaccgaccgcgacattgtgatcaaaggcctggctgcgggcctagacccgaataccgccacggc |
| | tggcgagaggcccgggacagcatctactacgtcgatgcgaacgcaagcatccaggagatgctcaacgtc |
| | atggaagaacatcaggtccgccgtgttccggtcatctcagagcaccgcttggtcggaatcgtcaccgaagc |
| | cgacatcgcccgacacctgcccgagcacgccattgtgcagttcgtcaaggcaatctgctcgcccatggccc |
| | tcgccagctag |
| | (SEQ ID NO: 29) |
| | ataaccacagcgcatgatatcatgaatgcgggtgtcacctgtgttggcgagcacgaaacgttgaccgcagc |
| | agcacagtacatgcgcgaacatgatatcggcgcattgccgatttgcggcgacgatgatcgtctgcacggtat |
| | gctgaccgaccacgatatcgttatcaagggtctggccgcaggcttggacccgaacaccgcgaccgccggt |
| | gaactggcacgtgacagcatctattacgtcgacgcgaacgccagcattcaagagatgctgaacgtgatgga |
| | agagcatcaggtgcgtcgtgtcccgattatcagcgaacatcgtctggttggtatcgttaccgaagccgacatc |
| | gcacgtcacctgccggagcacgcgattgttcagttcgtgaaagcgatttgcagcccgatggcgttggcgtc |
| | (SEQ ID NO: 30) |
| | acaacagccaggaacatcatgaacgccggcgtgacctgcgtgggagagcatgaaaccctcaccgccgcc |
| | gcccaatacatgagggagcacgacatcggcgccctgcccatctgtggagacgacgacaggctgcacggc |
| | atgctgaccgacagggacatcgtgatcaagggcctggctgccggcctcgatcctaacaccgctacagccg |
| | gcgagctggccagagacagcatctactacgtggacgccaacgccagcatccaggagatgctcaagtgat |
| | ggaggagcaccaggtgagaagggtgcctgtgatcagcgagcacaggctggtgggcatcgtgaccgagg |
| | ccgatatcgctaggcacctgcccgagcacgccatcgtgcagttcgtgaaggccatctgcagccccatggct |
| | ctggccagc |
| | (SEQ ID NO: 31) |
| | MTTARDIMNAGVTCVGEHETLTAAAQYMREHDIGALPICGDDDRL |
| | HGMLTDRDIVIKGLAAGLDPNTATAGELARDSIYYVDANASIQEML |
| | NVMEEHQVRRVPVISEHRLVGIVTEADIARHLPEHAIVQFVKAICSP |
| | MALAS |
| | (SEQ ID NO: 32) |
| Rv3407 | atgcgtgctaccgttgggcttgtggaggcaatcggaatccgagaactaagacagcacgcatcgcgatacct |
| | cgcccgggttgaagccggcgaggaacttggcgtcaccaacaaaggaagacttgtggcccgactcatcccg |
| | gtgcaggccgcggagcgttctcgcgaagccctgattgaatcaggtgtcctgattccggctcgtcgtccacaa |
| | aaccttctcgacgtcaccgccgaaccggcgcgcggccgcaagcgcaccctgtccgatgttctcaacgaaat |
| | gcgcgacgagcagtga |
| | (SEQ ID NO: 33) |
| | atgcgtgcgactgtgggtctggttgaggcgattggcattcgcgagctgcgccaacatgccagccgttacttg |
| | gctcgtgtcgaggcgggtgaagaactgggcgtgacgaataagggtcgtctggtcgcccgtctgattccggt |
| | tcaggcagctgagcattctcgcgaggcgctgattgaatccggcgtcctgatcccggctcgccgtccgcaaa |
| | acctgoggacgtgacggcggagccagctcgtggtcgcaaacgcacgctgEctgatgtcctgaacgaaatg |
| | cgcgacgagcag |
| | (SEQ ID NO: 34) |
| | ataagggcgaccgtcgggctagtggaagcgataggtatccggaagttgcgacagcacacatcacgatatc |
| | tggcacgggtggaagctggggaggaactgggcgtgaccaacaaggggcggctggtcgcgaggctgatc |
| | cccatgcaggccgccaagcggtcccgcaaagccctcatcaagtctggggtgctcattccagcacacagg |
| | ccgcaaaatctcctggacgtcactgcggagcccgccagaggcagaaagaggacgctgagtgacgtgctg |
| | aacgagatgagggacgaacag |
| | (SEQ ID NO: 35) |
| | MRATVGLVEAIGIRELRQHASRYLARVEAGEELGVTNKGRLVARLI |
| | PVQAAERSREALIESGVLIPARRPQNLLDVTAEPARGRKRTLSDVLN |
| | EMRDEQ |
| | (SEQ ID NO: 36) |
| Rv2628c | atgtccacgcaacgaccgaggcactccggtattcgggctgttggcccctacgcatgggccggccgatgtg |
| | gtcggataggcaggtggggggtgcaccaggaggcgatgatgaatctagcgatatggcacccgcgcaagg |
| | tgcaatccgccaccatctatcaggtgaccgatcgctcgcacgacgggcgcacagcacgggtgcctggtga |
| | cgagatcactagcaccgtgtccggttggttgtcggagttgggcacccaaagcccgttggccgatgagcttgc |
| | gcgtgcggtgcggatcggcgactggcccgctgcgtacgcaatcggtgagcacctgtccgttgagattgcc |
| | gttgcggtctaa |
| | (SEQ ID NO: 37) |
| | atgagcacccagagacccaggcacagcggcattagggccgtgggaccttatgcttgggccggcagatgc |
| | ggaaggatcggcagatggggcgtgcaccaagaggccatgatgaacctggccatctggcaccccaggaa |
| | ggtgcagagcgccaccatctaccaaggtgaccgacaggagccatgacggaagaccgccagagtgcccg |
| | gcgatgagatcaccagcaccgtgagcggctggctgagcgaactgggcacccaatcccccctggctgatga |
| | actggccagggctgtgaggatcggcgattggcctgccgcctatgccatcggcgagcatctgagcgtggag |
| | acgccgtggccgtgtaa |
| | (SEQ ID NO: 38) |
| | MSTQRPRHSGIRAVGPYAWAGRCGRIGRWGVHQEAMMNLAIWHP |
| | RKVQSATIYQVTDRSHDGRTARVPGDEITSTVSGWLSELGTQSPLAD |
| | ELARAVRIGDWPAAYAIGEHLSVEIAVAV |
| | (SEQ ID NO: 39) |

In some embodiments, the resuscitation Mtb antigen is RpfB, RpfD, or RpfE. In some embodiments, the resuscitation Mtb antigen is RpfB or RpfD. Additional resuscitation antigens include, but are not limited to, Rv0867c, Rv0288, Rv1009, Rv0685, Rv0824c, Rv2744c, Rv3347c, Rv1130, Rv1169c, Rv1884c, Rv2389c, and Rv2450c. In some embodiments, the resuscitation antigen is Rv0867c, Rv1884c, or Rv2389c. Additional resuscitation Mtb antigens are well known to the skilled artisan. In some embodiments, the resuscitation antigen is not any one or more of the following: Rv0867c, Rv0288, Rv1009, Rv0685, Rv0824c, Rv2744c, Rv3347c, Rv1130, Rv1169c, Rv1884c, Rv2389c, and Rv2450c. In some embodiments, the resuscitation antigen is not Rv0867c, Rv1884c, or Rv2389c.

The resuscitation Mtb antigen RpfB is also known as Rv1009. A nucleotide sequence encoding the resuscitation Mtb antigen RpfB is shown in Table 3 as SEQ ID NO:40 (mycobacterial sequence; not codon optimized) and SEQ ID NO:41 (mycobacterial sequence; signal sequence deleted), and amino acid sequences of RpfB corresponding thereto are shown in Table 3 as SEQ ID NO:42 and SEQ ID NO:43, respectively.

The resuscitation Mtb antigen RpfD is also known as Rv2389c. A nucleotide sequence encoding the resuscitation Mtb antigen RpfD is shown in Table 3 as SEQ ID NO:44 (mycobacterial sequence; not codon optimized), SEQ ID NO:45 (E. coli optimized; leader sequence deleted) and SEQ ID NO:46 (human optimized; leader sequence present), and amino acid sequences of RpfD are shown in Table 3 as SEQ ID NO:47 (E. coli optimized) and SEQ ID NO:48 (mycobacterial sequence and human optimized).

The resuscitation Mtb antigen RpfE is also known as Rv2450c. A nucleotide sequence encoding the resuscitation Mtb antigen RpfE is shown in Table 3 as SEQ ID NO:49 (mycobacterial sequence; not codon optimized), and an amino acid sequence of RpfE is shown in Table 3 as SEQ ID NO:50.

TABLE 3

| | nucleotide sequence |
|---|---|
| Construct | amino acid sequence |
| RpfB | atgttgcgcctggtagtcggtgcgctgctgctggtgttggcgttcgccggtggctatgcggtcgccgcatgca |
| | aaacggtgacgttgaccgtcgacggaaccgcgatgcgggtgaccacgatgaaatcgcgggtgatcgacatc |
| | gtcgaagagaacgggttctcagtcgacgaccgcgacgacctgtatcccgcggccggcgtgcaggtccatga |
| | cgccgacaccatcgtgctgcggcgtagccgtccgctgcagatctcgctggatggtcacgacgctaagcaggt |
| | gtggacgaccgcgtcgacggtggacgaggcgctggcccaactcgcgatgaccgacacggcgccggccgc |
| | ggcttctcgcgccagccgcgtcccgctgtccgggatggcgctaccggtcgtcagcgccaagacggtgcagc |
| | tcaacgacggcgggttggtgcgcacggtgcacttgccggcccccaatgtcgcggggctgctgagtgcggcc |
| | ggcgtgccgctgttgcaaagcgaccacgtggtgcccgccgcgacggccccgatcgtcgaaggcatgcaga |
| | tccaggtgacccgcaatcggatcaagaaggtcaccgagcggctgccgctgccgccgaacgcgcgtcgtgtc |
| | gaggacccggagatgaacatgagccgggaggtcgtcgaagacccgggggttccggggacccaggatgtg |
| | acgttcgcggtagctgaggtcaacggcgtcgagaccggccgtttgcccgtcgccaacgtcgtggtgacccc |
| | ggcccacgaagccgtggtgcgggtgggcaccaagcccggtaccgaggtgcccccggtgatcgacggaag |
| | catctgggacgcgatcgccggctgtgaggccggtggcaactgggcgatcaacaccggcaacgggtattac |
| | ggtggtgtgcagtttgaccagggcacctgggaggccaacggcgggctgcggtatgcaccccgcgctgacct |
| | cgccacccgcgaagagcagatcgccgttgccgaggtgacccgactgcgtcaaggttggggcgcctggccg |
| | gtatgtgctgcacgagcgggtgcgcgctga (SEQ ID NO: 40) |
| | gcatgcaaaacggtgacgttgaccgtcgacggaaccgcgatgcgggtgaccacgatgaaatcgcgggtgat |
| | cgacatcgtcgaagagaacgggttctcagtcgacgaccgcgacgacctgtatcccgcggccggcgtgcag |
| | gtccatgacgccgacaccatcgtgctgcggcgtagccgtccgctgcagatctcgctggatggtcacgacgct |
| | aagcaggtgtggacgaccgcgtcgacggtggacgaggcgctggcccaactcgcgatgaccgacacggcg |
| | ccggccgcggcttctcgcgccagccgcgtcccgctgtccgggatggcgctaccggtcgtcagcgccaaga |
| | cggtgcagctcaacgacggcgggttggtgcgcacggtgcacttgccggcccccaatgtcgcggggctgctg |
| | agtgcggccggcgtgccgctgttgcaaagcgaccacgtggtgcccgccgcgacggccccgatcgtcgaag |
| | gcatgcagatccaggtgacccgcaatcggatcaagaaggtcaccgagcggctgccgctgccgccgaacgc |
| | gcgtcgtgtcgaggacccggagatgaacatgagccgggaggtcgtcgaagacccgggggttccggggac |
| | ccaggatgtgacgttcgcggtagctgaggtcaacggcgtcgagaccggccgtttgcccgtcgccaacgtcgt |
| | ggtgaccccggcccacgaagccgtggtgcgggtgggcaccaagcccggtaccgaggtgcccccggtgat |
| | cgacggaagcatctgggacgcgatcgccggctgtgaggccggtggcaactgggcgatcaacaccggcaa |
| | cgggtattacggtggtgtgcagtttgaccagggcacctgggaggccaacggcgggctgcggtatgcacccc |
| | gcgctgacctcgccacccgcgaagagcagatcgccgttgccgaggtgacccgactgcgtcaaggttgggg |
| | cgcctggccggtatgtgctgcacgagcgggtgcgcgctga (SEQ ID NO: 41) |
| | MLRLVVGALLLVLAFAGGYAVAACKTVTLTVDGTAMRVTTMKSRV |
| | IDIVEENGFSVDDRDDLYPAAGVQVHDADTIVLRRSRPLQISLDGHDA |
| | KQVWTTASTVDEALAQLAMTDTAPAAASRASRVPLSGMALPVVSA |
| | KTVQLNDGGLVRTVHLPAPNVAGLLSAAGVPLLQSDHVVPAATAPI |
| | VEGMQIQVTRNRIKKVTERLPLPPNARRVEDPEMNMSREVVEDPGVP |
| | GTQDVTFAVAEVNGVETGRLPVANVVVTPAHEAVVRVGTKPGTEVP |
| | PVIDGSIWDAIAGCEAGGNWAINTGNGYYGGVQFDQGTWEANGGL |
| | RYAPRADLATREEQIAVAEVTRLRQGWGAWPVCAARAGAR (SEQ |
| | ID NO: 42) |
| | ACKTVTLTVDGTAMRVTTMKSRVIDIVEENGFSVDDRDDLYPAAGV |
| | QVHDADTIVLRRSRPLQISLDGHDAKQVWTTASTVDEALAQLAMTD |
| | TAPAAASRASRVPLSGMALPVVSAKTVQLNDGGLVRTVHLPAPNVA |
| | GLLSAAGVPLLQSDHVVPAATAPIVEGMQIQVTRNRIKKVTERLPLPP |
| | NARRVEDPEMNMSREVVEDPGVPGTQDVTFAVAEVNGVETGRLPV |
| | ANVVVTPAHEAVVRVGTKPGTFVPPVIDGSIWDAIAGCEAGGNWAI |
| | NTGNGYYGGVQFDQGTWEANGGLRYAPRADLATREEQIAVAEVTR |
| | LRQGWGAWPVCAARAGAR (SEQ ID NO: 43) |
| RpfD | atgacaccgggtttgcttactactgcgggtgctggccgaccacgtgacaggtgcgccaggatcgtatgcacg |
| | gtgttcatcgaaaccgccgttgtcgcgaccatgtttgtcgcgttgttgggtctgtccaccatcagctogaaagcc |
| | gacgacatcgattgggacgccatcgcgcaatgcgaatccggcggcaattgggcggccaacaccggtaacg |
| | ggttatacggtggtctgcagatcagccaggcgacgtgggattccaacggtggtgtcgggtcgccggcggcc |
| | gcgagtccccagcaacagatcgaggtcgcagacaacattatgaaaacccaaggcccgggtgcgtggccga |
| | aatgtagttcttgtagtcagggagacgcaccgctgggctcgctcacccacatcctgacgttcctcgcggccga |
| | gactggaggttgttcggggagcagggacgattga (SEQ ID NO: 44) |
| | aagcttttgctgggcctgagcaccattagcagcaaagcggatgacatcgactgggatgcgattgcgcagtgtg |
| | agagcggtggcaattgggcagcgaataccggcaatggcctgtacggcggtctgcagatctcccaggcgac |
| | gtgggacagcaatggtggcgtcggcagcccggctgccgcgtccccacaacaacagatcgaggtggcagat |
| | aacattatgaaaacgcagggtccgggtgcttggccaaaatgctccagctgcagccagggtgacgcaccgct |
| | gggcagcctgacccacattctgacgttcctggcagcggaaaccggtggttgtagcggtagccgcgatgac |
| | (SEQ ID NO: 45) |
| | acccccggactcctcaccacagctggagctggcaggcccagagacagatgcgccaggatcgtgtgcaccg |
| | tgttcatcgagaccgccgtggtggctaccatgttcgtggccctgctgggcctgagcaccatcagcagcaagg |
| | ccgacgacatcgactgggacgccatcgcccagtgtgaatccggcggaaactgggccgccaataccggcaa |
| | tggcctgtacggcggcctgcagatcagccaggctacctgggactccaacggaggagtgggaagccctgcc |
| | gctgcttcccctcagcagcagatcgaggtggccgacaacatcatgaagacccaaggccctggcgcctggcc |
| | taagtgttccagctgtagccagggcgatgctcctctgggcagcctgacccacatcctgacctttctcgccgccg |
| | agacaggcggatgtagcggaagcagggacgactaatga (SEQ ID NO: 46) |
| | LLGLSTISSKADDIDWDAIAQCESGGNWAANTGNGLYGGLQISQAT |
| | WDSNGGVGSPAAASPQQQIEVADNIMKTQGPGAWPKCSSCSQGDAP |
| | LGSLTHILTFLAAETGGCSGSRDD (SEQ ID NO: 47) |
| | TPGLLTTAGAGRPRDRCARIVCTVFIETAVVATMFVALLGLSTISSKA |
| | DDIDWDAIAQCESGGNWAANTGNGLYGGLQISQATWDSNGGVGSP |
| | AAASPQQQIEVADNIMKTQGPGAWPKCSSCSQGDAPLGSLTHILTFL |
| | AAETGGCSGSRDD (SEQ ID NO: 48) |
| RpfE | ttgaagaacgcccgtacgacgctcatcgccgccgcgattgccgggacgttggtgaccacgtcaccagccgg |
| | tatcgccaatgccgacgacgcgggcttggacccaaacgccgcagccggcccggatgccgtgggctttgacc |
| | cgaacctgccgccggccccggacgctgcacccgtcgatactccgccggctccggaggacgcgggctttgat |
| | cccaacctccccccgccgctggccccggacttcctgtccccgcctgcggaggaagcgcctcccgtgcccgt |
| | ggcctacagcgtgaactgggacgcgatcgcgcagtgcgagtccggtggaaactggtcgatcaacaccggta |
| | acggttactacggcggcctgcggttcaccgccggcacctggcgtgccaacggtggctcggggtccgcggc |
| | caacgcgagccgggaggagcagatccgggtggctgagaacgtgctgcgttcgcagggtatccgcgcctgg |
| | ccggtctgcggccgccgcggctga (SEQ ID NO: 49) |
| | LKNARTTLIAAAIAGTLVTTSPAGIANADDAGLDPNAAAGPDAVGFD |
| | PNLPPAPDAAPVDTPPAPEDAGFDPNLPPPLAPDFLSPPAEEAPPVPVA |
| | YSVNWDAIAQCESGGNWSINTGNGYYGGLRFTAGTWRANGGSGSA |
| | ANASREEQIRVAENVLRSQGIRAWPVCGRRG (SEQ ID NO: 50) |

In some embodiments, the fusion protein comprises at least four Mycobacterium tuberculosis (Mtb) antigens. In some embodiments, the fusion protein comprises at least five Mtb antigens. In some embodiments, the fusion protein comprises at least six Mtb antigens. In some embodiments, the fusion protein comprises from at least three to at least six Mtb antigens. In some embodiments, the fusion protein comprises from at least three to at least five Mtb antigens. In some embodiments, the fusion protein comprises at least three or at least four Mtb antigens. In some embodiments, the fusion protein comprises from at least four to at least six Mtb antigens. In some embodiments, the fusion protein comprises at least four or at least five Mtb antigens.

In some embodiments, the fusion protein comprises ESAT6, Rv1733c, Rv2626c, and RpfD Mtb antigens. In some embodiments, the fusion protein comprises ESAT6, Rv1733c, Rv2626c, and RpfB Mtb antigens. In some embodiments, the fusion protein comprises RpfB, ESAT6, Rv1733c, and Rv2626c Mtb antigens. In some embodiments, the fusion protein comprises Ag85B, ESAT6, Rv1733c, Rv2626c, and RpfD Mtb antigens. In some embodiments, the fusion protein comprises Ag85B, ESAT6, Rv1733c, Rv2626c, and RpfB Mtb antigens. In some embodiments, the fusion protein comprises PPE51, Rv1733c, Rv2628c, and RpfD Mtb antigens. In some embodiments, the fusion protein comprises PPE51, Rv1733c, Rv2628c, and RpfB Mtb antigens. In some embodiments, the fusion protein comprises Rv3407, Rv1733c, Rv2626c, and RpfB Mtb antigens. In some embodiments, the fusion protein comprises Rv3407, Rv1733c, Rv2626c, and RpfD Mtb antigens.

In any of the embodiments of fusion proteins set forth herein, the individual Mtb antigens can be present in any order. For example, for a fusion protein comprising ESAT6,

Rv1733c, Rv2626c, and RpfD Mtb antigens, the first (or N-terminal) antigen may be ESAT6, Rv1733c, Rv2626c, or RpfD; the second antigen may be ESAT6, Rv1733c, Rv2626c, or RpfD; the third antigen may be ESAT6, Rv1733c, Rv2626c, or RpfD; and the fourth (or C-terminal) antigen may be ESAT6, Rv1733c, Rv2626c, or RpfD. Likewise for every fusion protein disclosed herein.

Individual Mtb antigens may be linked together in a C-terminus to N-terminus manner without any linker (i.e., the C-terminus of ESAT6 linked directly to the N-terminus of Rv1733c). Alternately, a linker may be present between any two Mtb antigens within any of the fusion proteins disclosed herein. In some embodiments, the linker is a segment of DNA or RNA optionally containing one or more restrictions sites, wherein the linker is inserted between nucleic acid molecules encoding two Mtb antigens of any of the fusion proteins disclosed herein. Table 5 shows representative primers for particular Mtb antigens used to introduce restriction sites into a fusion protein construct.

In some embodiments, the fusion protein comprises ESAT6-Rv1733c-Rv2626c-RpfD (Construct A; nucleotide sequence is SEQ ID NO:51 (E. coli optimized; inserted EcoRI, SacI, and HindIII restriction sites, respectively, are bolded and underlined) and SEQ ID NO:52 (human optimized; inserted BstBI, PvuI, and AscI restriction sites, respectively, are bolded and underlined); corresponding amino acid sequences are SEQ ID NO:53 (E. coli optimized) and SEQ ID NO:54 (human optimized); see Table 4).

In some embodiments, the fusion protein comprises ESAT6-Rv1733c-Rv2626c-RpfB (Construct B; nucleotide sequence is SEQ ID NO:55, wherein inserted EcoRI, SacI, and HindIII restrictions sites, respectively, are bolded and underlined; amino acid sequence is SEQ ID NO:56; see Table 4).

In some embodiments, the fusion protein comprises RpfB-ESAT6-Rv1733c-Rv2626c (Construct C; nucleotide sequence is SEQ ID NO:57, wherein inserted BamHI, EcoRI, and SacI restrictions sites, respectively, are bolded and underlined; amino acid sequence is SEQ ID NO:58; see Table 4).

In some embodiments, the fusion protein comprises Ag85B-ESAT6-Rv1733c-Rv2626c-RpfD (Construct D; nucleotide sequence is SEQ ID NO:59 (E. coli optimized; inserted BamHI, EcoR1, SacI, and HindIII restriction sites, respectively, are bolded and underlined) and SEQ ID NO:60 (human optimized; inserted XmaI, BstBI, PvuI, and AscI restriction sites, respectively, are bolded and underlined); amino acid sequence is SEQ ID NO:61 (E. coli optimized) and SEQ ID NO:62 (human optimized); see Table 4).

In some embodiments, the fusion protein comprises PPE51-Rv1733c-Rv2628c-RpfD (Construct E; nucleotide sequence is SEQ ID NO:63, wherein inserted EcoRI, SacI, and HindIII restrictions sites, respectively, are bolded and underlined; amino acid sequence is SEQ ID NO:64; see Table 4).

In some embodiments, the fusion protein comprises PPE51-Rv1733c-Rv2628c-RpfB (Construct F; nucleotide sequence is SEQ ID NO:65, wherein inserted EcoRI, SalI, and HindIII restrictions sites, respectively, are bolded and underlined; amino acid sequence is SEQ ID NO:66; see Table 4).

In some embodiments, the fusion protein comprises Rv3407-Rv1733c-Rv2626c-RpfB (Construct G; nucleotide sequence is SEQ ID NO:67, wherein inserted EcoRI, SacI, and HindIII restrictions sites, respectively, are bolded and underlined; amino acid sequence is SEQ ID NO:68; see Table 4).

In some embodiments, the fusion protein comprises Rv3407-Rv1733c-Rv2626c-RpfD (Construct H; nucleotide sequence is SEQ ID NO:69, wherein inserted EcoRI, SacI, and HindIII restrictions sites, respectively, are bolded and underlined; amino acid sequence is SEQ ID NO:70; see Table 4).

In some embodiments, the fusion protein comprises PPE51-Rv1733c-Rv2626c-RpfD (Construct I; nucleotide sequence is SEQ ID NO:71, wherein inserted EcoRI, SacI, and HindIII restrictions sites, respectively, are bolded and underlined; amino acid sequence is SEQ ID NO:72; see Table 4).

In some embodiments, the fusion protein comprises PPE51-Rv1733c-Rv2626c-RpfB (Construct J; nucleotide sequence is SEQ ID NO:73, wherein inserted EcoRI, SacI, and HindIII restrictions sites, respectively., are bolded and underlined; amino acid sequence is SEQ ID NO: 74; see Table 4).

TABLE 4

| | nucleotide sequence |
|---|---|
| Construct | amino acid sequence |
| A | ataacagagcagcagtggaatttcgcgggtatcgaggccgcagcaagcgcaatccagggaaatgtcacgtc |
| | cattcattccctccttgacgaggggaagcagtccctgaccaagctcgcagcggcctggggcggtagcggttc |
| | ggaggcgtaccagggtgtccagcaaaaatgggacgccacggctaccgagctgaacaacgcgctgcagaa |
| | cctggcgcggacgatcagcgaagccggtcaggcaatggcttcgaccgaaggcaacgtcactgggatgttcg |
| | cagaattcatgattgcgactacccgtgatcgtgagggcgcgaccatgatcacgttccgtctacgtctgccgtgt |
| | cgcaccattttgcgcgtgttttcgcgtaacccgctggtccgcggtaccgaccgtctggaggcccccggggtcc |
| | aagacagccgtagccatgtgtatgctcaccaggctcaaacccgtcacccggctactgccactgttatcgatca |
| | cgaaggcgtgattgactccaataccacggcaacctccgcaccgcctcgcaccaagattacggttcctgcgcg |
| | agggtggtgaatggtattgaacgcagcggcgaagttaatgccaaaccgggtaccaaaagcggtgaccgtgt |
| | gggcatctgggtcgatagcgccggtcagctggtcgacgagccggcaccgccagcgcgtgcgatcgccgat |
| | tctagacgcgcaattctgatccgcgttcgcaatgcgagctggcagcacgatattgatagcctgttttgcacccaa |
| | cgtgagctcatgaccacggcgcgtgatatcatgaatgcgggtgtcacctgtgttggcgagcacgaaacgttg |
| | accgcagcagcacagtacatgcgcgaacatgatatcggcgcattgccgatttgcggcgacgatgatcgtctg |
| | cacggtatgctgaccgaccgcgatatcgttatcaagagtctggccgcaggcaggacccaaacaccgcgacc |
| | gccggtgaactggcacgtgacagcatctattacgtcgacgcgaacgccagcattcaagagatgctgaacgtg |
| | atggaagagcatcaggtgcgtcgtgtcccggttatcaacgaacatcgtctgattggtatcgttaccgaagcca |
| | acatcgcacgtcacctgccggagcacgcgattgacagttcgtgaaagcgatttgcagcccgatggcgttggc |
| | gtctaagcttttgctgggcctgagcaccattagcagcaaaacggatgacatcgactgggatgcgattacgca |
| | gtgtgagagcggtggcaattgggcagcgaataccggcaatggcctgtacggcggtctgcagatctcccagg |
| | cgacgtgggacagcaatggtggcgtcggcaacccggctgccacgtccccacaacaacagatcgaggtagc |
| | agataacattatgaaaacgcagggtccgggtgcttggccaaaatgctccagctgcagccagggtgacgcacc |
| | gctaggcaacctgacccacattctgacgttcctggcagcggaaaccggtggttatagcggtagccacgatga |
| | c = |
| | (SEQ ID NO: 51) |
| | atgaccgagcagcagtggaacttcgccggcatcgaagctgccgctagcgccatccaaggcaacgtgacca |
| | gcatccacagcctgctggacgagggcaagcagagcctgaccaagctggctgctgcttggggcggatccgg |
| | aagcgaagcctaccagggcgtgcagcagaagtgggacgccacagccaccgagctgaacaacgccctgca |
| | gaacctcgccagaaccatcagcgaggccggacaggctatggccagcacagagggcaatgtgaccggcat |
| | gttcgccttcgaaatcgccaccaccagggacagggaaggcgctaccatgatcaccttcaggctgaggctcc |
| | cctgcaggaccatcctgagggtgttcagcaggaaccccctggtgaggggcaccgacagactggaagccgt |
| | gcaggacagcaggagccacgtgtatgcccaccaggctcagaccaggcaccctgctaccgccaccgtgatc |
| | gaccacgagggcgtgatcgactccaaccaccgccaccagcgctcctcccagaaccaagatcacagtgc |
| | ccgccaggtgggtggtgaacggcatcgagaggagcggcgaggtgaacgccaagcctggaaccaagagc |
| | ggcgacagggtgggcatttgggtcgatagcgccggccagctggtggatgaacctgctccccctgccagagc |
| | catcgccgatagggccatcctgatcagggtgaggaacgccagctggcagcacgacatcgacagctgttct |
| | gcacccaaaggcgatcgacaacagccagggacatcatgaacgccggcgtgacctgcgtgggagagcatg |
| | aaaccctcaccaccgccgcccaatacatgaaggagcacgacatcggcgccctgcccatctgtagagacga |
| | cgacaggctgcacggcatgctgaccgacagggacatcgtgatcaagggcctggctgccggcctcgatccta |
| | acaccgctacagccaacgaactggccagagacagcatctactacgtggacaccaacaccagcatccagga |
| | gatgctcaacgtgatggaggagcaccaggtgagaagggtgcctgtgatcagcgagcacaggctggtgggc |
| | atcgtgaccgagaccgatatcgctaagcacctacccgagcacgccatcgtgcagttcgtgaaggccatctac |
| | agccccatggctctggccagcggcgcgcccacccccggactcctcaccacagctggagctggcaggccca |
| | gagacagatgcgccaggatcgtgtgaccgtgttcatcgagaccgccgtggtggctaccatattcgtggccct |
| | gctgggcctgagcaccatcagcagcaaggccgacgacatcgactgggacgccatcgcccagtgtgaatcc |
| | ggcggaaactgggccgccaataccggcaatggcctgtacggcggcctgcagatcagccaggctacctggg |
| | actccaacggaggagtgggaagccctgccgctgcttcccctcagcagcagatcgaggtggccgacaacatc |
| | atgaagacccaaggccctggcgcctggcctaagtgttccagctgtagccagggcgatgctcctctgggcagc |
| | ctgacccacatcctgacctttctcgccgccgagacaggcggatgtagcggaagcagggacgactaatgatag |
| | (SEQ ID NO: 52) |
| | MTEQQWNFAGIEAAASAIQGNVTSIHSLLDEGKQSLTKLAAAWGGS |
| | GSEAYQGVQQKWDATATELNNALNLARTISEAGQAMASTEGNVT |
| | GMFAEFMIATTRDREGATMITFRLRLPCRTILRVFSRNPLVRGTDRLE |
| | APGVQDSRSHVYAHQAQTRHPATATVIDHEGVIDSNTTATSAPPRTKI |
| | TVPARWVVNGIERSGEVNAKPGTKSGDRVGIWVDSAGQLVDEPAPP |
| | ADRAIADSRRAILIRVRNASWQHDIDSLFCTQRELMTTARDIMNAGVT |
| | CVGEHETLTAAAQYMREHDIGALPICGDDDRLHGMLTDRDIVIKGLA |
| | AGLDPNTATAGELARDSIYYVDANASIQEMLNVMEEHQVRRVPVISE |
| | HRLVGIVTEADIARHLPEHAIVQFVKAICSPMALASKLLLGLSTISSKA |
| | DDIDWDAIAQCESGGNWAANTGNGLYGGLQISQATWDSNGGVGSP |
| | AAASPQQQIEVADNIMKTQGPGAWPKCSSCSQGDAPLGSLTHILTFL |
| | AAETGGCSGSRDD |
| | (SEQ ID NO: 53) |
| | MTEQQWNEAGIEAAASAIQGNVTSIHSLLDEGKQSLTKLAAAWGGS |
| | GSEAYQGVQQKWDATATELNNALNLARTISEAGQAMASTEGNVTG |
| | MFAFEITTRDREGATMITFRLRLPCRTILRVFSRNPLVRGTDRLEAV |
| | QDSRSHVYAHQAQTRHPATATVIDHEGVIDSNTTATSAPPRTKITVPA |
| | RWVVNGIERSGEVNAKPGTKSGDRVGIWVDSAGQLVDEPAPPARAI |
| | ADRAILIRVRNASWQHDIDSLICTQRRSTTARDININAGVTCVGEHET |
| | LTAAAQYNIREHDIGALPICGDDDRLFIGNILTDRDIVIKGLAAGLDPNT |
| | ATAGELARDSIYYVDANASIQEMLNVMEEHQVRRVPVISEHRLVGIV |
| | TEADIARHLPEHAIVQFVKAICSPMALASGAPTPGLLTTAGAGRPRDR |
| | CARIVCTVFIETAVVATMFVALLGLSTISSKADDIDWDAIAQCESGGN |
| | WAANTGNGLYGGLQISQATWDSNGGVGSPAAASPQQQIEVADNIMK |
| | TQGPGAWPKCSSCSQGDAPLGSLTHILTFLAAETGGCSGSRDD |
| | (SEQ ID NO: 54) |
| B | atgacagagcagcagtggaatttcgcgggtatcgaggccgcggcaagcgcaatccagggaaatgtcacgtc |
| | cattcattccctccttgacgaggggaagcagtccctgaccaagctcgcagcgacctggggcggtagcggttc |
| | ggaggcgtaccagggtgtccagcaaaaatgggacgccacggctaccgagctgaacaacgcgctgcagaa |
| | cctggcgcggacgatcagcgaagccggtcaggcaatggcttcaaccgaaggcaacgtcactgggatgttcg |
| | cagaattcatgattgcgactacccgtgatcgtgagggcgcgaccatgatcacgttccgtctgcgtctgccgtgt |
| | cgcaccattttacgcgtgttttcgcgtaacccgctgatccgcggtaccgaccgtctggaggccgttatcatgct |
| | gctggcggttaccgtgagcctgctgacgatcccattcgcagcggcagctggcacggccgtccaagacagcc |
| | gtagccatgtgtatgctcaccaggctcaaacccgtcacccggctactgccactgttatcgatcacgaaggcgt |
| | gattgactccaataccacggcaacctccgcaccgcctcgcaccaagattacggttcctgcgcgttgggtggtg |
| | aatggtattgaacgcagcggcgaagttaatgccaaaccgggtaccaaaagcggtgaccatgtggacatctg |
| | ggtcgatagcgccggtcagctggtcgacgagccggcaccgccagcgcgtgcgatcgccgatgcggcgct |
| | ggctgccctgggtctgtggctgagcgtggcagcggtcgccggtgcgttgctggcgctgacgcgcgcaattct |
| | gatccgcgttcgcaatgcgagctggcagcacgatattgatagcctgttttgcacccaacgtgagctcatgacc |
| | acggcgcgtgatatcatgaatgcgggtgtcacctgtgttggcgagcacgaaacgttgaccgcagcagcaca |
| | gtacatgcgcgaacatgatatcggcgcattgccgatttgcggcgacgatgatcgtctgcacggtatgctgacc |
| | gaccgcgatatcgttatcaagggtctggccgcaggcttggacccgaacaccgcgaccgccggtgaactggc |
| | acgtgacagcatctattacgtcgacgcgaacgccagcattcaagagatgctgaacgtgatggaagagcatca |
| | ggtgcgtcgtgtcccggttatcagcgaacatcgtctggttggtatcgttaccgaagccgacatcgcacgtcacc |
| | tgccggagcacgcgattgttcagttcgtgaaagcgatttgcagcccgatggcgttggcgtctcgtcaaaaggg |
| | cgacacaaaatttattctaaatgcaaagcttgcatgcaaaacggtgacgttgaccgtcgacggaaccgcgatg |
| | cgggtgaccacgatgaaatcgcgggtgatcgacatcgtcgaagagaacgggttctcagtcgacgaccgcga |
| | cgacctgtatcccgcggccggcgtgcaggtccatgacgccgacaccatcgtgctgcggcgtagccgtccgc |
| | tgcagatctcgctggatggtcacgacgctaagcaggtgtggacgaccgcgtcgacggtggacgaggcgctg |
| | gcccaactcgcgatgaccgacacggcgccggccgcggcttctcgcgccagccgcgtcccgctgtccggga |
| | tggcgctaccggtcgtcagcgccaagacggtgcagctcaacgacggcgggttggtgcgcacggtgcacttg |
| | ccggcccccaatgtcgcggggctgctgagtgcggccggcgtgccgctgagcaaagcgaccacgtggtgc |
| | ccgccgcgacggccccgatcgtcgaaggcatgcagatccaggtgacccgcaatcggatcaagaaggtcac |
| | cgaacggctgccgctaccgccgaacgcgcgtcgtatcgaggacccaaagatgaacatgagccgggaggt |
| | cgtcgaagacccgggggttccggggacccaggatgtgacgttcgcggtagctgaggtcaacggcgtcgag |
| | accggccgtttgcccgtcgccaacgtcgtggtgaccccggcccacgaagccgtggtgcgggtgggcacca |
| | agcccggtaccgaggtgcccccggtgatcgacggaagcatctgggacgcgatcgccggctgtgaggccg |
| | gtggcaactgggcgatcaacaccggcaacgggtattacgatggtgtgcagtttgaccagaacacctaggag |
| | gccaacggcgggctgcggtatgcaccccgcgctgacctcgccacccgcgaagagcagatcgccgttgccg |
| | aggtgacccgactgcgtcaaggttggggcgcctggccagtatgtgctgcacgagcgggtgcgcgctga |
| | (SEQ ID NO: 55) |
| | MTEQQWNFAGIEAAASAIQGNVTSIHSLLDEGKQSLTKLAAAWGGS |
| | GSEAYQGVQQKWDATATELNNALQNLARTISEAGQAMASTEGNVT |
| | GMFAEFMIATTRDREGATMITFRLRLPCRTILRVFSRNPLVRGTDRLE |
| | AVVMLLAVTVSLLTIPFAAAAGTAVQDSRSHVYAHQAQTRHPATAT |
| | VIDHEGVIDSNTTATSAPPRTKITVPARWVVNGIERSGEVNAKPGTKS |
| | GDRVGIWVDSAGQLVDEPAPPARAIADAALAALGWLSVAAVAGA |
| | LLALTRAILIRVRNASWQHDIDSLFCTQRELMTTARDIMNAGVTCVG |
| | EHETLTAAAQYMREHDIGALPICGDDDRLHGMLTDRDIVIKGLAAGL |
| | DPNTATAGELARDSIYYVDANASIQEMLNVMEEHQVRRVPVISEHRL |
| | VGIVTEADIARHLPEHAIVQFVKAICSPMALASRQKGDTKFILNAKLA |
| | CKTVTLTVDGTAMRVTTMKSRVIDIVEENGFSVDDRDDLYPAAGVQ |
| | VHDADTIVLRRSRPLQISLDGHDAKQVWTTASTVDEALAQLAMTDT |
| | APAAASRASRVPLSGMALPVVSAKTVQLNDGGLVRTHLPAPNVAG |
| | LLSAAGVPLLQSDHVVPAATAPIVEGMQIQVTRNRIKKVTERLPLPPN |
| | ARRVEDPEMNMSREVVEDPGVPGTQDVTFAVAEVNGVETGRLPVA |
| | NVVVTPAHEAVVRVGTKPGTEVPPVIDGSTWDAIAGCEAGGNWAINT |
| | GNGYYGGVQFDQGTWEANGGLRYAPRADLATREEQIAVAEVTRLR |
| | QGWGAWPVCAARAGAR |
| | (SEQ ID NO: 56) |
| C | atgaagcttgcatgcaaaacggtgacgttgaccgtcgacggaaccgcgatgcgggtgaccacgatgaaatc |
| | gcgggtgatcgacatcgtcgaaaagaacgggttctcagtcaacgaccgcgacgacctatatcccgcggccg |
| | gcgtgcaggtccatgacgccgacaccatcgtgctgcggcgtagccgtccgctgcagatctcgctggatggtc |
| | acgacgctaaacaggtgtggacgaccgcgtcgacgatggacgaggcgctggcccaactcgcgatgaccg |
| | acacggcgccggccgcggcactcgcgccagccgcgtcccgctgtccgggatggcgctaccggtcgtcag |
| | cgccaagacagtgcagctcaacgacggcgggttagtgcacacggtgcacttgccggcccccaatgtcgca |
| | gggctgctgagtgcggccggcgtgccgctgttgcaaagcgaccacgtggtgcccgccgcgacggccccg |
| | atcgtcgaaggcatgcagatccaggtgacccgcaatcggatcaagaaggtcaccgagcggctgccgctgcc |
| | gccgaacgcgcgtcgtgtcgaggacccggagatgaacatgagccgggaggtcgtcgaagacccgggggt |
| | tccggggacccaggatgtgacgttcgcggtagctgaggtcaacggcgtcgagaccggccgtttgcccgtcg |
| | ccaacgtcgtggtgaccccggcccacgaagccgtggtgcgggtgggcaccaagcccggtaccgaggtgc |
| | ccccggtgatcgacggaagcatctgggacgcgatcgccggctgtgaggccggtggcaactgggcgatcaa |
| | caccggcaacgggtattacggtggtgtgcagtttgaccagggcacctgggaggccaacggcgggctgcgg |
| | tatgcaccccgcgctgacctcgccacccgcgaagagcagatcgccgttgccgaggtgacccgactgcgtca |
| | aggttggggcgcctggccggtatgtgctgcacgagcgggtgcgcgcggatccatgacagagcagcagtgg |
| | aatttcgcgggtatcgaggccgcggcaagcgcaatccagggaaatgtcacgtccattcattccctccttgacg |
| | aggggaagcagtccctgaccaagctcgcagcggcctggggcgatagcggttcggaggcgtaccagggtgt |
| | ccagcaaaaatgggacgccacggctaccgagctgaacaacgcgctgcagaacctggcgcggacgatcag |
| | cgaagccggtcaggcaatggcttcgaccgaaggcaacgtcactgggatattcgcagaattcatgattgcgac |
| | tacccgtgatcgtgagggcgcgaccatgatcacgttccgtctgcgtctgccgtgtcgcaccattttgcgcgtgtt |
| | ttcgcgtaacccgctggtccgcggtaccgaccgtctggaggccgttgtcatgctgctggcggttaccgtgagc |
| | ctgctgacgatcccattcgcagcggcagctggcacggccgtccaagacagccgtagccatgtgtatgctcac |
| | caggctcaaacccgtcacccggctactgccactgttatcgatcacgaaggcgtgattgactccaataccacgg |
| | caacctccgcaccgcctcgcaccaagattacggttcctgcgcgttgggtggtgaatggtattgaacgcagcg |
| | gcgaagttaatgccaaaccgggtaccaaaagcggtgaccgtgtgggcatctgggtcgatagcgccggtcag |
| | ctggtcgacgagccggcaccgccagcgcgtgcgatcgccgatacggcactggctgccctgggtctgtaact |
| | gagcgtggcagcggtcgccggtgcgttgctggcgctgacgcgcgcaattctgatccgcgttcgcaatgcga |
| | gctggcagcacgatattgatagcctOttacacccaacgtgagctcatgaccacggcgcgtgatatcatgaat |
| | gcgggtgtcacctgtgttggcgagcacgaaacgttgaccgcagcagcacagtacatgcgcgaacatgatatc |
| | ggcgcattgccgatttgcggcgacaatgatcatctgcacggtatgctgaccgaccgcgatatcgttatcaaag |
| | gtctggccgcaggcttggacccgaacaccgcgaccgccggtgaactggcacgtgacagcatctattacgtc |
| | gacgcgaacgccagcattcaagagatgctgaacgtgatggaagagcatcaggtgcgtcgtgtcccggttatc |
| | agcgaacatcgtctggttggtatcgttaccgaagccgacatcgcacgtcacctgccggagcacgcgattgac |
| | agttcgtgaaagcgatttgcagcccgatggcgttggcgtctcgtcaaaagggcgacacaaaatttattctaaat |
| | gcatga |
| | (SEQ ID NO: 57) |
| | MKLACKTVTLTVDGTAMRVTTMKSRVIDIVEENGFSVDDRDDLYPA |
| | AGVQVHDADTIVLRRSRPLQISLDGHDAKQVWTTASTVDEALAQLA |
| | MTDTAPAAASRASRVPLSGMALPVVSAKTVQLNDGGLVRTVHLPAP |
| | NVAGLLSAAGVPLLQSDHVVPAATAPIVEGMQIQVTRNRIKKVTERL |
| | PLPPNARRVEDPEMNMSREVVEDPGVPGTQDVTFAVAEVNGVETGR |
| | LPVANVVVTPAHEAVVRVGTKPGTEVPPVIDGSIWDAIAGCEAGGN |
| | WAINTGNGYYGGVQFDQGTWEANGGLRYAPRADLATREEQIAVAE |
| | VTRLRQGWGAWPVCAARAGARGSMTEQQWNFAGIEAAASAIQGNV |
| | TSIHSLLDEGKQSLTKLAAAWGGSGSEAYQGVQQKWDATATELNNA |
| | LQNLARTISEAGQAMASTEGNVTGMFAEFMIATTRDREGATMITFRL |
| | RLPCRTILRVFSRNPLVRGTDRLEAVVMLLAVTVSLLTIPFAAAAGTA |
| | VQDSRSHVYAHQAQTRHPATATVIDHEGVIDSNTTATSAPPRTKITVP |
| | ARWVVNGIERSGEVNAKPGTKSGDRVGIWVDSAGQLVDEPAPPARA |
| | IADAALAALGLWLSVAAVAGALLALTRAILIRVRNASWQHDIDSLFC |
| | TQRELMTTARDIMNAGVTCVGEHETLTAAAQYMREHDIGALPICGD |
| | DDRLHGMLTDRDIVIKGLAAGLDPNTATAGELARDSIYYVDANASIQ |
| | EMLNVMEEHQVRRVPVISEHRLVGIVTEADIARHLPEHAIVQFVKAIC |
| | SPMALASRQKGDTKFILNA |
| | (SEQ ID NO: 58) |
| D | atgatagccgtcctggcctgccagttgaatacctgcaagttccgagcccgtccatgggtcgtgacattaaggt |
| | gcaattccaaagcgacggtaacaatagcccggctgtgtacctactggacggtctacgtgcgcaggataatta |
| | caacggctgggacatcaataccccggcatttgagtggtattaccagtcgggtctgagcattgtgatgccggttg |
| | gcggtcaaagcagcttctatagcgattggtacagcccggcatgcggcaaggctgattgccaaacctacaagt |
| | gggaaactttcttgaccagcgagctgccgcaatggttgagcgccaaccgtgcggtcaaaccgaccggt |
| | agcgctgctattggcctgtccatggccggcagcagcgcgatgatcttggcggcataccatccgcagcaattta |
| | tctacgccggtagcctgagcgcattgctggacccgagccaaggcatgggtccgagcctgattggtctggcaa |
| | tgggtgacgcaggtggttacaaagcggccgatatgtggggcccatctagcgacccggcatgggagcgtaat |
| | gacccgacccagcaaattccgaaactggtggcgaataacacgcgcctgtgggtctactgtggcaatggtacg |
| | ccgaacgagctaggtggcgcgaatatccctgcggagtttctggaaaactttgttcgcagcagcaacctgaaat |
| | tccaggacgcgtataacgcagccggtggtcacaatgcggttttcaatttcccgccaaatggcactcatagctgg |
| | gagtactggggtgcgcagttgaacgcaatgaaaggcgatctgcaatcctctctgggtgcgggcggatccatg |
| | acagagcagcagtggaatttcgcgggtatcgaggccgcggcaagcgcaatccagggaaatgtcacgtccat |
| | tcattccctccttgacgaggggaagcagtccctgaccaagctcgcagcggcctggggcggtagcggttcgg |
| | aggcgtaccagggtgtccagcaaaaatgggacgccacggctaccgagctgaacaacgcgctgcagaacct |
| | ggcgcggacgatcagcgaagccggtcaggcaatggcttcgaccgaaggcaacgtcactgggatgttcgca |
| | gaattcatgattgcgactacccgtgatcgtgagggcgcgaccatgatcacgaccgtctgcgtctgccgtgtc |
| | gcaccattttgcgcatgttttcgcgtaacccgctaatccgcggtaccgaccgtctggaggcccccggggtcca |
| | agacagccgtagccatgtgtatgctcaccaggctcaaacccgtcacccggctactgccactgttatcgatcac |
| | gaaagcgtgattgactccaataccacggcaacctccgcaccacctcgcaccaaaattacaattcctacgcgtt |
| | gggtggtgaatggtattgaacgcagcggcgaagttaatgccaaaccgggtaccaaaagcggtgaccgtgtg |
| | ggcatctgggtcgatagcgccggtcagctggtcgacaagccggcaccaccagcgcatgcaatcgccaatt |
| | ctagacgcgcaattctgatccgcgttcgcaatgcgagctggcagcacgatattgatagcctgttttgcacccaa |
| | cgtgagctcatgaccacggcgcgtgatatcatgaatgcgggtgtcacctgtgttggcgagcacgaaacgttg |
| | accgcagcagcacagtacatgcgcgaacatgatatcggcgcattgccgatttgcggcgacgatgatcgtctg |
| | cacgatatgctgaccgaccgcgatatcgttatcaagggtctggccgcaggcttggacccgaacaccgcgacc |
| | gccggtgaactggcacgtgacagcatctattacgtcgacgcgaacgccagcattcaagagatgctgaacgtg |
| | atggaagagcatcaggtgcgtcgtgtcccggttatcagcgaacatcgtctggttggtatcgttaccgaagccg |
| | acatcgcacgtcacctgccggagcacgcgattgttcagttcgtgaaagcgatttgcagcccgatggcgttggc |
| | gtctaagcttttgctgggcctgagcaccattagcagcaaagcggatgacatcgactgggatgcgattgcgca |
| | gtgtgagagcggtggcaattgggcagcgaataccggcaatggcctgtacggcggtctgcagatctcccagg |
| | cgacgtgggacagcaatggtggcgtcggcagcccggctgccgcgtccccacaacaacagatcgaggtggc |
| | agataacattatgaaaacgcagggtccgggtgcttggccaaaatgctccagctgcagccagggtgacgcacc |
| | gctgggcagcctgacccacattctgacgttcctggcagcggaaaccggtggttgtagcggtagccgcgatga |
| | c |
| | (SEQ ID NO: 59) |
| | atgttctccaggcccggcctgcctgtcgagtatctgcaggtcccctccccctccatgggcagagacatcaagg |
| | tgcagttccaatccggaggcaacaacagccccgccgtgtatctcctcgacggcctgagggctcaggacgact |
| | acaacggctgggacatcaacacccccgccttcgagtggtactaccagtccggactgagcatcgtcatgcccg |
| | tgggcggccagagctccttctacagcgactggtatagccctgcctgcggcaaagccggatgccagacctaca |
| | agtaagagacctttctgaccagcgaactgccccagtggctgtccgccaatagggccgtcaaacctaccggct |
| | ccgctgccatcggactcagcatggccggaagctccgctatgatcctggccgcctaccacccccagcaatttat |
| | ctacgctagcagcctgtccgctctgctggatcctagccaaggcataagccctagcctcattggcctggccatg |
| | ggcgatgctggcggctataaggccgccgatatgtggggccctagctccgatcctgcctgggagaggaatga |
| | ccccacccagcagatccccaagctggtaaccaacaacacaaggctctgggtgtactgcaacaatggcaccc |
| | ccaacgaactgggcggagccaacattcccgccgagacctggagaacttcgtcaggagcagcaacctgaag |
| | ttccaggacgcctacaatgccgccggaggccacaacgctgtgttcaacttccctcccaacgacacccacagc |
| | tgggagtattggggcgctcagctgaacgccatgaaaggcgacctccagagctccctgggagctggacccg |
| | ggaccgagcagcagtggaacttcgccggcatcgaagctgccgctagcgccatccaaggcaacgtgaccag |
| | catccacagcctgctggacgagggcaagcagagcctgaccaagctggctgctgcttggggcggatccgga |
| | agcgaagcctaccagggcgtgcagcagaagtgggacgccacagccaccgagctgaacaacgccctgcag |
| | aacctcgccagaaccatcagcgaggccggacaggctatggccagcacagagggcaatgtgaccggcatgt |
| | tcgccttcgaaatcgccaccaccagggacagggaaggcgctaccatgatcaccttcaggctgaggctcccc |
| | tgcaggaccatcctgagggtgacagcaggaaccccaggtgaggggcaccgacagactggaagccgtgc |
| | aggacagcaggagccacgtgtatgcccaccaggctcagaccaggcaccctgctaccgccaccgtgatcga |
| | ccacgagggcgtgatcgactccaacaccaccgccaccagcgctcctcccagaaccaagatcacagtgccc |
| | gccaggtgggtggtgaacggcatcgagaggagcggcgaggtgaacgccaagcctggaaccaagagcgg |
| | cgacagggtgggcatagggtcgatagcgccggccagctggtggatgaacctgctccccctgccagagcca |
| | tcgccgatagggccatcctgatcagggtgaggaacgccagctggcagcacgacatcgacagcctgttctgc |
| | acccaaaggcgatcgacaacagccagggacatcatgaacgccggcgtgacctgcgtgggagagcatgaa |
| | accctcaccgccgccgcccaatacatgagggagcacgacatcggcgccctgcccatagtggagacgacg |
| | acaggctgcacggcatgctgaccgacagggacatcgtgatcaagggcctggctgccggcctcgatcctaac |
| | accgctacagccggcgagctggccagagacagcatctactacgtggacgccaacgccagcatccaggaga |
| | tgctcaacgtgatggaggagcaccaggtgagaagggtgcctgtgatcagcgagcacaggctggtgggcatc |
| | gtgaccgaggccgatatcgctaggcacctgcccgagcacgccatcgtgagttcgtgaaggccatagcag |
| | ccccatggctctggccagcggcgcgcccacccccggactcctcaccacagctggagctggcaggcccaga |
| | gacagatgcgccaggatcgtgtgaccgtgttcatcaagaccgccgtgatggctaccatgttcgtggccctgc |
| | tgggctgagcaccatcagagcaaggcgacgacatcgactgggacgccatcgcccagtgtgaatccgg |
| | cggaaactgggccgccaataccggcaatggcctgtacggcggcctgagatcaaccaggctacctgggact |
| | ccaacggaggagtgggaagccctgccgctgcttcccctcagcagcagatcgaggtggccgacaacatcatg |
| | aagacccaagaccctggcacctggcctaagtgttccagctgtagccagggcgatgctcctctgggcagcctg |
| | acccacatcctgacctttctcgccgccgagacaggcggatgtagggaagcagggacgactaatgatag |
| | (SEQ ID NO: 60) |
| | MFSRPGLPVEYLQVPSPSMGRDIKVQFQSGGNNSPAVYLLDGLRAQD |
| | DYNGWDINTPAFEWYYQSGLSIVMPVGGQSSFYSDWYSPACGKAGC |
| | QTYKWETFLTSELPQWLSANRAVKPTGSAAIGLSMAGSSANILAAY |
| | HPQQFIYAGSLSALLDPSQGMGPSLIGLAMGDAGGYKAADMWGPSS |
| | DPAWERNDPTQQIPKLVANNTRLWVYCGNGTPNELGGANIPAEFLE |
| | NFVRSSNLKFQDAYNAAGGHNAVFNFPPNGTHSWEYWGAQLNAMK |
| | GDLQSSLGAGGSMTEQQWNFAGIEAAASAIQNVSIHSILLDEGKQS |
| | LTKLAAAWGGSGSEAYQGVQQKWDATATELNNALQNLARTISEAG |
| | QAMASTEGNVTGMFAEFMIATTRDREGATMITFRLRLPCRTILRVFSR |
| | NPLVRGTDRLEAPGVQDSRSHVYAHQAQTRHPATATVIDHEGVIDSN |
| | TTATSAPPRTKITVPARWVVNGIERSGEVNAKPGTKSGDRVGIWVDS |
| | AGQLVDEPAPPARAIADSRRAILIRVRNASWQHDIDSLFCTQRELMTT |
| | ARDIMNAGVTCVGEHETLTAAAQYMREHDIGALPICGDDDRLHGML |
| | TDRDIVIKGLAAGLDPNTATAGELARDSIYYVDANASIQEMLNVMEE |
| | HQVRRVPVISEHRLVGIVTEADIARHLPEHAIVQFVKAICSPMALASK |
| | LLLGLSTISSKADDIDWDAIAQCESGGNWAANTGNGLYGGLQISQAT |
| | WDSNGGVGSPAAASPQQQIEVADNIMKTQGPGAWPKCSSCSQGDAP |
| | LGSLTHILTFLAAETGGCSGSRDD |
| | (SEQ ID NO: 61) |
| | MFSRPGLPVEYLQVPSPSMGRDIKVQFQSGGNNSPAVYLLDGLRAQD |
| | DYNGWDINTPAFEWYYQSGLSIVMPVGGQSSFYSDWYSPACGKAGC |
| | QTYKWETFLTSELPQWLSANRAVKPTGSAAIGLSMAGSSAMILAAY |
| | HPQQFIYAGSLSALLDPSQGMGPSLIGLAMGDAGGYKAADMWGPSS |
| | DPAWERNDPTQQIPKLVANNTRLWVYCGNGTPNELGGANIPAEFLE |
| | NFVRSSNLKFQDAYNAAGGHNAVFNFPPNGTHSWEYWGAQLNAMK |
| | GDLQSSLGAGPGTEQQWNFAGIEAAASAIQGNVTSIHSLLDEGKQSL |
| | TKLAAAWGGSGSEAYQGVQQKWDATATELNNALQNLARTISEAGQ |
| | AMASTEGNVTGMFAFEIATTRDREGATMITFRLRLTCRTILRVFSRNP |
| | LVRGTDRLEAVQDSRSHVYAHQAQTRHPATATVIDHEGVIDSNTTAT |
| | SAPPRTKITVPARWVVNGIERSGEVNAKPGTKSGDRVGIWVDSAGQL |
| | VDEPAPPARAIADRAILIRVRNASWQHDIDSLFCTQRRSTTARDIMNA |
| | GVTCVGEHETLTAAAQYMREHDIGALPICGDDDRLHGMLTDRDIVIK |
| | GLAAGLDPNTATAGELARDSIYYVDANASIQEMLNVMEEHQVRRVP |
| | VISEHRLVGIVTEADIARHLPEHAIVQFVKAICSPMALASGAPTPGLLT |
| | TAGAGRPRDRCARIVCTVFIETAVVATMFVALLGLSTISSKADDIDW |
| | DAIAQCESGGNWAANTGNGLYGGLQISQATWDSNGGVGSPAAASPQ |
| | QQIEVADNIMKTQGPGAWPKCSSCSQGDAPLGSLTHILTFLAAETGG |
| | CSGSRDD |
| | (SEQ ID NO: 62) |
| E | atggattttgcgctgctgccgccggaagtgaacagcgcgcgcatgtataccggcccgggcgcgggcagcct |
| | gctggcggcggcgggcggctgggatagcctggcggcggaactggcgaccaccgcggaagcgtatggca |
| | gcgtgctgagcggcctggcggcgctgcattggcgcggcccggcggcggaaagcatggcggtgaccgcg |
| | gcgccgtatattggctggctgtataccaccgcggaaaaaacccagcagaccgcgattcaggcgcgcgcggc |
| | ggcgctggcgtttgaacaggcgtatgcgatgaccctgccgccgccggtggtggcggcgaaccgcattcagc |
| | tgctggcgctgattgcgaccaactttttggccagaacaccgcggcgattgcggcgaccgaagcgcagtatg |
| | cggaaatgtgggcgcaggatacggcgacgatgtatggctatacgaccgcgagcgcggcagcggcgctgc |
| | tgaccccgtttagcccgccgcgccagaccaccaacccggcgggcctgaccgcgcaggcggcggcggtga |
| | gccaaacgaccgatccgctgagcctgctgattgaaaccgtgacccaggcgctgcaggcgctgaccattccg |
| | agctttattccggaagattttacctttctggatgcgatttttgcgggctatgcgaccgtgggcgtgacccaggatg |
| | tggaaagctttgtggcgggcaccattggcgcggaaagcaacctggacctgctaaacgtgggcgataaaaac |
| | ccggcggaagtgaccccgggcgattttggcattggcgaactggtgagcgcgaccagcccgggcggcggc |
| | gtgagcacgagcggcgcgggcggcgcggcgagcgtgggcaacaccgtactggcgagcgtgggccgcg |
| | cgaacagcattggccagctgagcgtgccgccgagogggcggcgccgagcacccgcccggtgagcgcgc |
| | tgaacccggcgggcctaaccaccctgccaggcaccgatgtagcggaacatggcatgccgggcgtgccgg |
| | gcgtgccggtggcggcgggccgcgcgagcggcgtgctgccgcgctatggcgtgcgcctgaccgtgatgg |
| | cgcatccgccgacggcgggcgaattcatgattgcgactacccgtgatcatgagggcgcgaccatgatcacg |
| | ttccgtagcgtctgccgtgtcgcaccattttgcgcgtgttttcgcgtaacccgctggtccgcggtaccgaccgt |
| | ctggaggccgttgtcatgctgctggcggttaccgtgagcctgctgacgatcccattcgcagcggcagctggc |
| | acggccgtccaagacagccgtagccatgtgtatgctcaccaggctcaaacccgtcacccggctactgccact |
| | gttatcgatcacgaaggcgtgattgactccaataccacggcaacctccgcaccgcctcgcaccaagattacgg |
| | ttcctgcgcgttgggtggtgaatggtattgaacgcagcggcgaagttaatgccaaaccgggtaccaaaagcg |
| | gtgaccgtgtgggcatctgggtcgatagcgccggtcagctggtcgacgagccggcaccgccagcgcgtgc |
| | gatcgccgatgcggcgctggctgccctgggtctgtggctgagcgtggcagcggtcgccggtgcgttgctgg |
| | cgctgacgcgcgcaattctgatccgcgttcgcaatgcgagctggcagcacgatattgatagcctgttttgcacc |
| | caacgtgagctcatgtccacgcaacgaccgaggcactccggtattcgggctgttggcccctacgcatgggcc |
| | ggccgatgtggtcggataggcaggtggggggtgcaccaggaggcgatgatgaatctagcgatatggcacc |
| | cgcacaaggtgcaatccgccaccatctatcaggtgaccgatcgctcgcacgacgggcgcacagcacgggt |
| | gcctggtgacgagatcactagcaccgtgtccggttggttgtcggagttgggcacccaaagcccgttggccga |
| | taagcttgcgcgtacggtacggatcggcgactggcccgctgcatacgcaatcggtgagcacctgtccgttga |
| | gattgccgttgcggtcaagcttttagctgggcctgagcaccattagcagcaaagcggatgacatcgactgggat |
| | gcgattgcgcagtgtaagagcggtggcaattgggcagcgaataccggcaatggcctgtacggcggtctgca |
| | gatctcccaggcgacgtgggacagcaatggtggcgtcggcagcccggctgccgcgtccccacaacaacag |
| | atcgaggtggcagataacattatgaaaacgcagggtccgggtgcttggccaaaatgctccagctgcagccag |
| | ggtgacgcaccgctgggcagcctgacccacattctgacgttcctggcagcggaaaccggtggttgtagcggt |
| | agccgcgatgactga |
| | (SEQ ID NO: 63) |
| | MDFALLPPEVNSARMYTGPGAGSLLAAAGGWDSLAAELATTAEAY |
| | GSVLSGLAALHWRGPAAESMAVTAAPYIGWLYTTAEKTQQTAIQAR |
| | AAALAFEQAYAMTLPPVVAANRIQLLALIATNFFGQNTAAIAATEA |
| | QYAEMWAQDAAAMYGYATASAAAALLTPFSPPRQTTNPAGLTAQA |
| | AAVSQATDPLSLLIETVTQALQALTIPSFIPEDFTFLDAIFAGYATVGV |
| | TQDVESFVAGTIGAESNLGLLNVGDENPAEVTPGDFGIGELVSATSPG |
| | GGVSASGAGGAASVGNTVLASVGRANSIGQLSVPPSWAAPSTRPVSA |
| | LSPAGLTTLPGTDVAEHGMPGVPGVPVAAGRASGVLPRYGVRLTVM |
| | AHPPAAGEFEFMIATTRDREGATMITFRLRLPCRTILRVFSRNPLVRGT |
| | DRLEAVVMLLAVTVSLLTIPFAAAAGTAVQDSRSHVYAHQAQTRHP |
| | ATATVIDHEGVIDSNTTATSAPPRTKITVPARWVVNGIERSGEVNAKP |
| | GTKSGDRVGIWVDSAGQLVDEPAPPARAIADAALAALGLWLSVAAV |
| | AGALLALTRAILIRVRNASWQHDIDSLFCTQRELMSTQRPRHSGIRAV |
| | GPYAWAGRCGRIGRWGVHQEAMMNLAIWHPRKVQSATIYQVTDRS |
| | HDGRTARVPGDEITSTVSGWLSELGTQSPLADELARAVRIGDWPAAY |
| | AIGEHLSVEIAVAVKLLLGLSTISSKADDIDWDAIAQCESGGNWAAN |
| | TGNGLYGGLQISQATWDSNGGVGSPAAASPQQQIEVADNIMKTQGP |
| | GAWPKCSSCSQGDAPLGSLTHILTFLAAETGGCSGSRDD |
| | (SEQ ID NO: 64) |
| F | ataaattttacgctgctaccgccggaagtaaacagcgcgcacatgtataccggcccggacgcggacagcct |
| | gctggcggcggcgggcggctgggatagcctggcggcggaactggcgaccaccgcggaagcgtatggca |
| | gcgtgctgagcggcctggcggcgctgcattaacgcgacccggcggcggaaagcatggcggtgaccgcg |
| | gcgccgtatattggctggctgtataccaccgcggaaaaaacccagcagaccgcgattcaggcgcgcgcggc |
| | ggcgctggcatttgaacaggcgtatgcaatgaccctgccgccgccggtggtggcggcgaaccgcattcagc |
| | tgctggcgctgattgcgaccaactttatggccagaacaccgcggcgattgcggcgaccgaagcgcagtatg |
| | cggaaatgtgggcgcaggatgcgacggcgatgtataactatgcaaccgcgagcgcagcggcagcgctgc |
| | tgaccccgtttagcccgccgcgccagaccaccaacccggcgggcctgaccgcgcaggcggcggcggtga |
| | gccaggcaaccgatccgctgagcctgctgattgaaaccgtgacccaggcgctacaggcgctgaccattccg |
| | agctttattccggaagatatacctttctggatgcgaatttgcgggctatgcgaccgtgggcgtgacccaggatg |
| | tagaaagctttatggcggacaccattggcgcggaaaacaacctgggcctgctgaacgtgggcgatgaaaac |
| | ccggcggaagtgaccccgggcgattttggcattggcgaactggtgagcgcgaccagcccgggcggcggc |
| | gtgagcgcgagcggcgcgggcggcgcggcgagcgtgggcaacaccgtgctggcgagcgtgggccgcg |
| | cgaacagcattggccagctgagcgtgccgccgagctgggcggcgccgagcacccgcccggtgagcgcgc |
| | tgagcccggcgggcctgaccaccctgccgggcaccgatgtggcggaacatggcatgccgggcgtgccgg |
| | gcgtgccggtggcggcgggccgcgcgagcggcgtgctgccgcgctatggcgtgcgcctgaccgtgatgg |
| | cgcatccgccggcggcgggcgaattcatgattgcgactacccgtgatcgtgagggcgcgaccatgatcacg |
| | ttccgtctgcgtctgccgtgtcgcaccattttgcgcgtgttttcgcgtaacccgctggtccgcggtaccgaccgt |
| | ctggaggccgagtcatgctgctggcggttaccgtgagcctgctgacgatcccattcgcagcggcagctggc |
| | acggccgtccaagacagccgtagccatgtgtatgctcaccaggctcaaacccgtcacccggctactgccact |
| | gttatcgatcacgaaggcgtgattgactccaataccacggcaacctccgcaccgcctcgcaccaagattacgg |
| | ttcctgcgcattgggtggtgaatagtattgaacgcagcggcgaagttaatgccaaaccgggtaccaaaagcg |
| | gtgaccgtgtgggcaEctgggtcgatagcgccggtcagctggtcgacgagccggcaccgccagcgcgtgc |
| | gatcgccgatgcaacgctggctgccctgggtctgtggctgagcgtggcagcggtcaccggtgcgttgctgg |
| | cgctgacgcgcgcaattctgatccgcgttcgcaatgcgagctggcagcacgatattgatagcctgttttgcacc |
| | caacgtgagctcatgtcatatccacgcaacgaccgaggcactccggtattcgggctgttggcccctacgcatgggcc |
| | ggccgatgtggtcggataggcaggtggggggtgcaccaggaggcgatgatgaatctagcgatatggcacc |
| | cgcgcaaggtgcaatccgccaccatctatcaggtgaccgatcgctcgcacaacgaacgcacaacacgggt |
| | gcctggtgacgagatcactagcaccgtgtccggttggttgtcggagttgggcacccaaagcccgttggccga |
| | tgaacttgcgcgtgcaatgcgaatcggcgactgacccgctacgtacgcaatcggtgagcacctgtccgttga |
| | gattgccgttgcggtcaagcttgcatgcaaaacggtgacgttgaccgtcgacggaaccgcgatgcgggtga |
| | ccacgatgaaatcgcgggtgatcgacatcgtcgaagagaacgggttctcagtcgacgaccgcgacgacctg |
| | tatcccgcggccggcgtgcaggtccatgacgccgacaccatcgtgctgcggcgtagccgtccgctgcagat |
| | ctcgctggatggtcacgacgctaagcaggtgtggacgaccgcgtcgacggtggacgaggcgctggcccaa |
| | ctcgcgatgaccgacacggcgccggccgcggcttctcgcgccagccgcgtcccgctgtccgggatggcgc |
| | taccggtcgtcagcgccaagacggtgcagctcaacgacggcgggttggtgcgcacggtgcacttgccggc |
| | ccccaatgtcgcggggctgctgagtgcggccggcgtgccgctgttgcaaagcgaccacgtggtgcccgcc |
| | gcgacggccccgatcgtcgaaggcatgcagatccaggtgacccgcaatcggatcaagaaggtcaccgagc |
| | ggctgccgctgccgccgaacgcgcgtcgtgtcgaggacccggagatgaacatgagccgggaggtcgtcg |
| | aagacccgggggttccggggacccaggatgtgacgttcgcggtagctgaggtcaacggcgtcgagaccgg |
| | ccgtttgcccgtcgccaacgtcgtggtgaccccggcccacgaagccgtggtgcgggtgggcaccaagccc |
| | ggtaccgaggtgcccccggtgatcgacggaagcatctgggacgcgatcgccggctgtgaggccggtggca |
| | actgggcgatcaacaccggcaacgggtattacggtggtgtgcagtttgaccagggcacctgggaggccaac |
| | ggcgggctgcggtatgcaccccgcgctgacctcgccacccgcgaagagcagatcgccgttgccgaggtga |
| | cccgactgcgtcaaggttggggcgcaggccggtatgtgctgacgagcgggtgcgcgctga |
| | (SEQ ID NO: 65) |
| | MDFALLPPEVNSARMYTGPGAGSLLAAAGGWDSLAAELATTAEAY |
| | GSVLSGLAALHWRGPAAESMAVTAAPYIGWLYTTAEKTQQTAIQAR |
| | AAALAFEQAYAMTLPPVVAANRIQLLALIATNFFGQNTAAIAATEA |
| | QYAEMWAQDAAAMYGYATASAAAALLTPFSPPRQTTNPAGLTAQA |
| | AAVSQATDPLSLLIETVTQALQALTIPSFIPEDFTFLDAIFAGYATVGV |
| | TQDVESFVAGTIGAESNLGLLNVGDENPAEVTPGDFGIGELVSATSPG |
| | GGVSASGAGGAASVGNTVLASVGRANSIGQLSVPPSWAAPSTRPVSA |
| | LSPAGLTTLPGTDVAEHGMPGVPGVPVAAGRASGVLPRYGVRLTVM |
| | AHPPAAGEFMIATTRDREGATMITFRLRLPCRTILRVFSRNPLVRGTD |
| | RLEAVVMLLAVTVSLLTIPFAAAAGTAVQDSRSHVYAHQAQTRHPA |
| | TATVIDHEGVIDSNTTATSAPPRTKITVPARWVVNGIERSGEVNAKPG |
| | TKSGDRVGIWVDSAGQLVDEPAPPARAIADAALAALGLWLSVAAVA |
| | GALLALTRAILIRVRNASWQHDIDSLFCTQRELMSTQRPRHSGIRAVG |
| | PYAWAGRCGRIGRWGVHQEAMMNLAIWHPRKVQSATIYQVTDRSH |
| | DGRTARVPGDEITSTVSGWLSELGTQSPLADELARAVRIGDWPAAYA |
| | IGEHLSVEIAVAVKLACKTVTLTVDGTAMRVTTMKSRVIDIVEENGF |
| | SVDDRDDLYPAAGVQVHDADTIVLRRSRPLQISLDGHDAKQVWTTA |
| | STVDEALAQLAMTDTAPAAASRASRVPLSGMALPVVSAKTVQLNDG |
| | GLVRTVHLPAPNVAGLLSAAGVRLLQSDHVVPAATAPIVEGMQIQVT |
| | RNRIKKVTERLPLPPNARRVEDPEMNMSREVVEDPGVPGTQDVTFAV |
| | AEVNGVETGRLPVANVVVTPAHEAVVRVGTKPGTEVPPVIDGSIWD |
| | AIAGCEAGGNWAINTGNGYYGGVQFDQGTWEANGGLRYAPRADLA |
| | TREEQIAVAEVTRLRQGWGAWPVCAARAGAR |
| | (SEQ ID NO: 66) |
| G | atgcgtgcgactatgggtctggttgaagcgattggcattcgcgagctgcgccaacatgccagccgttacttaa |
| | ctcgtgtcgaggcgggtgaagaactgggcgtgacgaataagggtcgtctggtcgcccgtctgattccggttca |
| | ggcagctgagcgttctcgcgaggcgctaattgaatccggcgtcctgatcccggctcgccgtccgcaaaacct |
| | gctggacgtgacggcggagccagctcgtggtcgcaaacgcacgcttgtctgatgtcctgaacgaaatgcgcg |
| | acgagcaggaattcatgattgcgactacccgtaatcgtgagggcgcgaccatgatcacgttccgtctgcgtct |
| | gccgtgtcgcaccattttagcgcgtgattttcgcgtaacccgctggtccgcggtaccgaccgtctggaggccgttg |
| | tcatgctgctggcggttaccgtgagcctactgacgatcccattcgcagcggcagctggcacggccgtccaag |
| | acagccgtagccatgtgtatgctcaccaggctcaaacccgtcacccggctactgccactgttatcgatcacga |
| | aggcgtaattgactccaataccacggcaacctccgcaccacctcgcaccaaaattacaattcctacgcgttgg |
| | gtggtgaatggtattgaacgcagcggcgaagttaatgccaaaccgggtaccaaaagcggtgaccgtgtggg |
| | catctgggtcaatagcgccggtcagctggtcgacgagccggcaccgccagcacgtgcgatcgccgatgcg |
| | gcgctggctgccctgagtctgtggctgagcgtggcagcggtcgccggtgcgttgctggcgctgacgcgcgc |
| | aattctgatccgcgttcgcaatgcgagctggcagcacgatattgatagcctgttttgcacccaacgtgagctcat |
| | gaccacggcgcgtgatatcatgaatgcgggtgtcacctgtgttggcgagcacgaaacgttgaccgcagcag |
| | cacagtacatgcgcgaacatgatatcggcgcattgccgatttgcggcgacgatgatcgtctgcacggtatgct |
| | gaccgaccgcgatatcgttatcaagggtctggccgcaggcttggacccgaacaccgcgaccgccggtgaac |
| | tggcacgtgacagcatctattacgtcgacgcgaacgccagcattcaagagatgctgaacgtgatggaagagc |
| | atcaggtgcgtcgtgtcccggttatcagcgaacatcgtaggttggtatcgttaccgaagccgacatcgcacgt |
| | cacctgccggagcacgcgattgttcagttcgtgaaagcgatttgcagcccgatggcgttggcgtctcgtcaaa |
| | agggcgacacaaaatttattctaaatgcaaagccttgcatgcaaaacggtgacgttgaccgtcgacggaaccg |
| | cgatgcgggtgaccacgatgaaatcgcgggtgatcgacatcgtcgaagagaacgggttctcagtcgacgac |
| | cgcgacgacctgtatcccgcggccggcgtgcaggtccatgacgccgacaccatcgtgctgcggcgtagcc |
| | gtccgctgcagatctcgctggatggtcacgacgctaagcaggtgtggacgaccgcgtcgacggtggacgag |
| | gcgctggcccaactcgcgatgaccgacacggcgccggccgcggcttctcgcgccagccgcgtcccgctgt |
| | ccgggatggcgctaccggtcgtcagcgccaagacggtgcagctcaacgacggcgggttggtgcgcacggt |
| | gcacttgccggcccccaatgtcgcggggctgctgagtgcggccggcgtgccgctgttgcaaagcgaccac |
| | gtggtgcccgccgcgacggccccgatcgtcgaaggcatgcagatccaggtgacccgcaatcggatcaaga |
| | aggtcaccgagcggctgccgctgccgccgaacgcgcgtcgtgtcgaggacccggagatgaacatgagcc |
| | gggaggtcgtcgaagacccaagggttccggggacccagaatgtgacattcgcggtagctgaggtcaacgg |
| | cgtcgagaccggccgtttgcccgtcgccaacgtcgtggtgaccccggcccacgaagccgtggtgcgggtg |
| | ggcaccaagcccggtaccaaggtacccccagtgatcgacggaagcatctgggacgcgatcaccggctgtg |
| | aggccggtggcaactgggcgatcaacaccggcaacgggtattacggtggtgtgcagtttgaccagggcacc |
| | tgggaggccaacggcgggctgcagtatgcaccccgcgctaacctcaccacccgcgaagagcaaatcgcc |
| | gttgccgaggtgacccgactgcgtcaaggttggggcgcctggccggtatgtgctgcacgagcgggtgcgcg |
| | ctga |
| | (SEQ ID NO: 67) |
| | MRATVGLVEAIGIRELRQHASRYLARVEAGEELGVTNKGRLVARLIP |
| | VQAAERSREALIESGVLIPARRPQNLLDVTAEPARGRKRTLSDVLNE |
| | MRDEQEFMIATTRDREGATMITFRLRLPCRTILRVFSRNPLVRGTDRL |
| | EAVVMLLAVTVSLLTIPFAAAAGTAVQDSRSHVYAHQAQTRHPATA |
| | TVIDHEGVIDSNTTATSAPPRTKITVPARWVVNGIERSGEVNAKPGTK |
| | SGDRVGIWVDSAGQLNDEPAPPARAIADAALAALGLWLSVAAVAGA |
| | LLALTRAILIRVRNASWQHDIDSLFCTQRELMTTARDIMNAGVTCVG |
| | EHETLTAAAQYMREHDIGALPICGDDDRLHGMLTDRDIVIKGLAAGL |
| | DPNTATAGELARDSIYYVDANASIQEMLNVMEEHQVRRVPVISEHRL |
| | VGIVTEADIARHLTERAIVQFVKAICSPMALASRQKGDTKFILNAKLA |
| | CKTVTLTVDGTAMRVTTMKSRVIDIVEENGFSVDDRDDLYPAAGVQ |
| | VHDADTIVLARRSRPLQISLDGHDAKQVWTTASTVDEALAQLAMTDT |
| | APAAASRASRVPLSGMALPVVSAKTVQLNDGGLVRTVHLPAPNVAG |
| | LLSAAGVPLLQSDHVVPAATAPIVEGMQIQVTRNRIKKVTERLPLPPN |
| | ARRVEDPEMNMSREVVEDPGVPGTQDVTFAVAEVNGVETGRLPVA |
| | NVVVTPAHEAVVRVGTKPGTEVPPVIDGSIWDAIAGCEAGGNWAINT |
| | GNGYYGGVQFDQGTWEANGGLRYAPRADLATREEQIAVAEVTRLR |
| | QGWGAWPVCAARAGAR |
| | (SEQ ID NO: 68) |
| H | atgcgtgcgactatgggtctggttgaagcgattggcattcgcgagctgcgccaacatgccagccgttacttaa |
| | ctcgtgtcgaggcgggtgaagaactgggcgtgacgaataagggtcgtctggtcgcccgtctgattccggttca |
| | ggcagctgagcgttctcgcgaggcgctaattgaatccggcgtcctgatcccggctcgccgtccgcaaaacct |
| | gctggacgtgacggcggagccagctcgtggtcgcaaacgcacgctgtctgatgtcctgaacgaaatgcgcg |
| | acgagcaggaattcatgattgcgactacccgtaatcgtgagggcgcgaccatgatcacgttccgtctgcgtct |
| | gccgtgtcgcaccattagcgcgtgattcgcgtaacccgctggtccgcggtaccgaccgtctggaggccgttg |
| | tcatgctgctggcggttaccgtgagcctactgacgatcccattcgcagcggcagctggcacggccgtccaag |
| | acagccgtagccatgtgtatgctcaccaggctcaaacccgtcacccggctactgccactgttatcgatcacga |
| | aggcgtgattgactccaataccacggcaacctccgcaccgcctcgcaccaagattacggttcctgcgcgttgg |
| | gtggtgaatggtattgaacgcagcggcgaagttaatgccaaaccgggtaccaaaagcggtgaccgtgtggg |
| | catctgggtcgatagcgccggtcagctggtcgacgagccggcaccgccagcgcgtgcgatcgccgatgcg |
| | gcgctggctgccctgggtctgtggctgagcgtggcagcggtcgccggtgcgttgctggcgctgacgcgcgc |
| | aattctgatccgcgttcgcaatgcgagctggcagcacgatattgatagcctgattgcacccaacgtgagctcat |
| | gaccacggcgcgtgatatcatgaatgcgggtgtcacctgtgttggcgagcacgaaacgttgaccgcagcag |
| | cacagtacatgcgcgaacatgatatcggcgcattgccgatttgcggcgacgatgatcgtctgcacggtatgct |
| | gaccgaccgcgatatcgttatcaagggtctggccgcaggcttagacccgaacaccgcgaccgccggtgaac |
| | tggcacgtgacagcatctattacgtcgacgcgaacgccagcattcaagagatgctgaacgtgatggaagagc |
| | atcaggtgcgtcgtgtcccggttatcagcgaacatcgtctggttggtatcgttaccgaagccgacatcgcacgt |
| | cacctgccggagcacgcgattgttcagttcgtgaaagcgatttgcagcccgatggcgttggcgtctcgtcaaa |
| | agggcgacacaaaatttattctaaatgcaaagcttttgctgggcctgagcaccattagcagcaaagcggatga |
| | catcgactgggatgcgattgcgcagtgtgagagcggtggcaattgggcagcgaataccggcaatggcctgt |
| | acggcggtctgcagatctcccaggcgacgtgggacagcaatggtggcgtcggcagcccggctgccgcgtc |
| | cccacaacaacagatcgaggtggcagataacattatgaaaacgcagggtccgggtgcttggccaaaatgctc |
| | cagctgcagccagggtgacgcaccgctgggcagcctgacccacattctgacgttcctggcagcggaaaccg |
| | gtggttgtagcggtagccgcgatgactga |
| | (SEQ ID NO: 69) |
| | MRATVGLVEAIGIRELRQRASRYLARVEAGEELGVTNKGRLVARLIP |
| | VQAAERSREALIESGVLIPARRPQNLLDVTAEPARGRKRTLSDVLNE |
| | MRDEQEFMIATTRDREGATMITFRLRLPCRTILRVFSRNPLVRGTDRL |
| | EAVVMLLAVTVSLLTIPFAAAAGTAVQDSRSHVYAHQAQTRHPATA |
| | TVIDHEGVIDSNTTATSAPPRTKITVPARWVVNGIERSGEVNAKPGTK |
| | SGDRVGIWVDSAGQLVDEPAPPARAIADAALAALGLWLSVAAVAGA |
| | LLALTRAILIRVRNASWQHDIDSLFCTQRELMTTARDIMNAGVTCVG |
| | EHETLTAAAQYMREHDIGALPICGDDDRLHGMLTDRDIVIKGLAAGL |
| | DPNTATAGELARDSIYYVDANASIQEMLNVMEEHQVRRVPVISEHRL |
| | VGIVTEADIARHLPEHAIVQFVKAICSPMALASRQKGDTKFILNAKLL |
| | LGLSTISSKADDIDWDAIAQCESGGNWAANTGNGLYGGLQISQATW |
| | DSNGGVGSPAAASPQQQIEVADNIMKTQGPGAWPKCSSCSQGDAPL |
| | GSLTHILTFLAAETGGCSGSRDD |
| | (SEQ ID NO: 70) |
| I | atggattttgcgctgctgccgccggaagtgaacagcgcgcgcatgtataccggcccgggcgcgggcagcct |
| | gctggcggcggcgggcggctgggatagcctggcggcggaactggcgaccaccgcggaagcgtatggca |
| | gcgtgctgagcggcctggcggcgctgcattggcgcggcccggcggcggaaagcatggcggtgaccgcg |
| | gcgccgtatattggctggctgtataccaccgcggaaaaaacccagcagaccgcgattcaggcgcgcgcggc |
| | ggcgctggcgtttgaacaggcgtatgcgatgaccctgccgccgccggtggtggcggcgaaccgcattcagc |
| | tgaggcgctgattgcgaccaacttttttggccagaacaccgcggcgattgcggcgaccgaagcgcagtatg |
| | cggaaatgtgggcgcaggatgcggcggcgatgtatggctatgcgaccgcgagcgcggcggcggcgctgc |
| | tgaccccgtttagcccgccgcgccagaccaccaacccggcgggcctgaccgcgcaggcggcggcggtga |
| | gccaggcgaccgatccgctgagcctgctgattgaaaccgtgacccaggcgctgcaggcgctgaccattccg |
| | aptttattccagaagattttacctttctggatgcgatttttgcgaactatpaaccgtgggcgtgacccaggatg |
| | tggaaagattgtggcgggcaccattggcgcggaaagcaacctggpctgctgaacgtgggcgatgaaaac |
| | ccggcaaaagtgaccccgggcgattttggcattggcgaactggtaagcgcgaccaacccgggcggcggc |
| | gtgagcgcgagcggcgcgggcggcgcggcgagcgtgggcaacaccgtgctggcgagcgtgggccgcg |
| | cgaacagcattggccaactgagcgtgccgccgagctgggcggcgccgagcacccgcccggtgagcgcgc |
| | tgagcccggcgggcctgaccaccctgccgggcaccgatgtggcggaacatggcatgccgggcgtgccgg |
| | gcgtgccggtgacggcgaaccgcgcgagcggcgtgctgccgcgctatggcgtgcgcctgaccgtgatgg |
| | cgcatccgccggcggcgggcgaatttatgacagagcagcagtggaatttcgcgggtatcgaggccgcggc |
| | aaacgcaatccagggaaatgtcacgtccattcattccctccttgacgaggggaagcagtccctgaccaagctc |
| | gcagcggcctggggcggtagcggttcggaggcgtaccagggtgtccagcaaaaatgggacgccacggct |
| | accgagctgaacaacgcgctgcagaacctggcgcggacgatcagcgaagccggtcaggcaatggcttcga |
| | ccgaaggcaacgtcactgggatgttcgcagaattcatgattgcgactacccgtgatcgtgagggcgcgacca |
| | tgatcacgttccgtctgcgtctgccgtgtcgcaccattttgcgcgtgttttcgcgtaacccgctggtccgcggtac |
| | cgaccgtctggaggccgttgtcatgctgctggcggttaccgtgagcctgctgacgatcccattcgcagcggca |
| | gctggcacggccgtccaagacagccgtagccatgtgtatgctcaccaggctcaaacccgtcacccggctact |
| | gccactgttatcgatcacgaaggcgtgattgactccaataccacggcaacctccgcaccgcctcgcaccaag |
| | attacggttcctgcgcgttgggtggtgaatggtattgaacgcagcggcgaagttaatgccaaaccgggtacca |
| | aaagcggtgaccgtgtgggcatctgggtcgatagcgccggtcagctggtcgacgagccggcaccgccagc |
| | gcgtgcgatcgccgatgcggcgctggctgccctgggtctgtggctgagcgtggcagcggtcgccggtgcgt |
| | tgctggcgctgacgcgcgcaattctgatccgcgttcgcaatgcgagctggcagcacgatattgatagcctgttt |
| | tgcacccaacgtgagctcatgaccacggcgcgtgatatcatgaatgcgggtgtcacctgtgttggcgagcac |
| | gaaacgttgaccgcagcagcacagtacatgcgcgaacatgatatcggcgcattgccgatttgcggcgacgat |
| | gatcgtctgcacggtatgctgaccgaccgcgatatcgttatcaagggtctggccgcaggcttggacccgaac |
| | accgcgaccgccggtgaactggcacgtgacagcatctattacgtcgacgcgaacgccagcattcaagagat |
| | gctgaacgtgatggaagagcatcaggtgcgtcgtgtcccggttatcagcgaacatcgtctggaggtatcgtta |
| | ccgaagccgacatcgcacgtcacctgccggagcacgcgattgttcagttcgtgaaagcgatttgcagcccga |
| | tggcgttgacgtctcatcaaaagggcaacacaaaatttattctaaatgcaaagcttttgctgggcctaagcacc |
| | attagcagcaaagcggatgacatcgactgggatgcgattgcgcagtgtgagagcggtggcaattgggcagc |
| | gaataccagcaatggcctatacggcggtctgcagatctcccaaacgacgtgggacaacaatggtggcgtcg |
| | gcagcccggctgccgcgtccccacaacaacagatcgaggtggcagataacattatgaaaacgcagggtccg |
| | ggtgcttggccaaaatgctccagctgcagccagggtgacacaccgctgggcagcctgacccacattctgac |
| | gttcctggcagcggaaaccggtggttgtagcggtagccgcgatgactga |
| | (SEQ ID NO: 71) |
| | MDEALLPPEVNSARMYTGPGAGSLLAAAGGWDSLAAELATTAEAY |
| | GSVLSGLAALHWRGPAAESMAVTAAPYIGWYLYTTAEKTQQTAIQAR |
| | AAALAFEQAYAMTLPPPVVAANRIQLLALIATNFFGQNTAAIAATEA |
| | QYAEMWAQDAAAMYGYATASAAAALLTPFSPPRQTTNPAGLTAQA |
| | AAVSQATDPLSLLIETVTQALQALTIPSFIPEDFTFLDAIFAGYATVGV |
| | TQDVESFVAGTIGAESNLGLLNVGDENPAEVTPGDFGIGELVSATSPG |
| | GGVSASGAGGAASVGNTVLASVGRANSIGQLSVPPSWAAPSTRPVSA |
| | LSPAGLTTLPGTDVAEHGMPGVPGVPVAAGRASGVLPRYGVRLTVM |
| | AHPPAAGEFMIATTRDREGATMITFRLRLPCRTILRVFSRNPLVRGTD |
| | RLEAVVMLLAVTVSLLTIPFAAAAGTAVQDSRSHVYAHQAQTRHPA |
| | TATVIDHEGVIDSNTTATSAPPRTKITVPARWVVNGIERSGEVNAKPG |
| | TKSGDRVGIWVDSAGQLVDEPAPPARAIADAALAALGLWLSVAAVA |
| | GALLALTRAILIRVRNASWQHDIDSLFCTQRELMTTARDIMNAGVTC |
| | VGEHETLTAAAQYMREHDIGALPICGDDDRLHGMLTDRDIVIKGLAA |
| | GLDPNTATAGELARDSIYYVDANASIQEMLNVMEEHQVRRVPVISEH |
| | RLVGIVTEADIARHLPEHAIVQFVKAICSPMALASRQKGDTKFILNAK |
| | LLLGLSTISSKADDIDWDAIAQCESGGNWAANTGNGLYGGLQISQAT |
| | WDSNGGVGSPAAASPQQQIEVADNIMKTQGPGAWPKCSSCSQGDAP |
| | LGSLTHILTFLAAETGGCSGSRDDKMK |
| | (SEQ ID NO: 72) |
| J | atggattttgcgctgctgccgccggaagtgaacagcgcacgcatgtataccggcccgggcgcgggcagcct |
| | gctggcggcggcgggcggctgggatagcctggcggcggaactggcgaccaccgcggaagcgtatggca |
| | gcgtgctgagcggcctggcggcgctgcattggcacggcccggcggcggaaaacatggcggtgaccgcg |
| | gcgccgtatattggctggctgtataccaccgcggaaaaaacccagcagaccgcgattcaggcgcgcgcggc |
| | ggcgctagcgtttgaacaggcgtatacgatgaccctgccgccgccggtggtggcggcgaaccgcattcagc |
| | tgctggcgctgattgcgaccaacttttttggccagaacaccgcggcgattgcggcgaccgaagcgcagtatg |
| | cggaaatgtgggcgcaggatacggcgacgatgtatggctatacgaccgcgagcgcggcagcggcgctgc |
| | tgaccccgatagcccgccgcgccagaccaccaacccggcgggcctgaccgcgcaggcggcggcggtga |
| | gccaggcgaccgatccgctgagcctgctgattgaaaccgtgacccaggcgctgcaggcgctgaccattccg |
| | agctttattccggaagattttacctttctggatgcgatttttgcgggctatgcgaccgtgggcgtgacccaggatg |
| | tggaaagctttgtggcgggcaccattggcgcggaaagcaacctgggcctgctgaacgtgggcgatgaaaac |
| | ccggcggaagtgaccccgggcgattttggcattggcgaactggtgagcgcgaccagcccgggcggcggc |
| | gtgagcgcgagcggcgcgggcggcgcggcgagcgtgggcaacaccgtgctggcgagcgtgggccgcg |
| | cgaacagcattggccagctgagcgtgccgccgagctgggcggcgccgagcacccgcccggtgagcgcgc |
| | tgagcccggcgggcctgaccaccctgccgggcaccgatgtggcggaacatggcatgccgggcgtgccgg |
| | gcgtgccggtggcggcgggccgcgcgagcggcgtgctgccgcgctatggcgtgcgcctgaccgtgatgg |
| | cgcatccgccggcggcgggcgaatttatgacagagcagcagtggaatttcgcgggtatcgaggccgcggc |
| | aagcgcaatccagggaaatgtcacgtccattcattccctccttgacgaggggaagcagtccctgaccaagctc |
| | gcagcggcctggggcggtagcggttcggaggcgtaccagggtgtccagcaaaaatgggacgccacggct |
| | accgagctgaacaacgcgctgcagaacctggcgcggacgatcagcgaagccggtcaggcaatggcttcga |
| | ccgaaggcaacgtcactgggatgttcgcagaattcatgattgcgactacccgtgatcgtgagggcgcgacca |
| | tgatcacgttccgtctgcgtctgccgtgtcgcaccattttgcgcgtgttttcgcgtaacccgctggtccgcggtac |
| | cgaccgtctggaggccgttgtcatgctgctggcggttaccgtgagcctgctgacgatcccattcgcagcggca |
| | gctggcacggccgtccaagacagccgtagccatgtgtatgctcaccaggctcaaacccgtcacccggctact |
| | gccactgttatcgatcacgaaaacgtaattgactccaataccacggcaacctccgcaccacctcgcaccaaa |
| | attacggttcctgcgcgttgggtggtgaatggtattgaacgcagcggcgaagttaatgccaaaccgggtacca |
| | aaagcggtgaccatgtgaacatctaagtcaatagcgccggtcagctggtcgacgagccggcaccgccagc |
| | gcgtgcgatcgccgatgcggcgctggctgccctgggtctgtggctgagcgtggcagcggtcgccggtgcgt |
| | tgctggcgctgacgcacgcaattctgatccgcgttcgcaatgcgagctggcagcacgatattgataacctgttt |
| | tgcacccaacgtgagctcatgaccacggcgcgtgatatcatgaatgcgggtgtcacctgtgttggcgagcac |
| | gaaacgttgaccgcagcaacacagtacatgcgcgaacatgatatcggcacattgccgatttacggcgacgat |
| | gatcgtctgcacggtatgctgaccgaccgcgatatcgttatcaagggtctggccgcaggcaggacccgaac |
| | accgcgaccgccggtgaactggcacgtgacagcatctattacgtcgacgcgaacgccagcattcaagagat |
| | gctgaacgtgatggaagagcatcaggtgcgtcgtgtcccggttatcagcgaacatcgtctggttggtatcgtta |
| | ccgaagccgacatcgcacgtcacctgccggagcacgcgattgttcagttcgtgaaagcgatttgcagcccga |
| | tggcgttggcgtctcgtcaaaagggcgacacaaaatttattctaaatgcaaagcttgcatgcaaaacggtgac |
| | gttgaccgtcgacggaaccgcgatgcgggtgaccacgatgaaatcgcgggtgatcgacatcgtcgaagaga |
| | acgggttctcagtcgacgaccgcgacgacctgtatcccgcggccggcgtgcaggtccatgacgccgacacc |
| | atcgtgctgcggcgtagccgtccgctgcagatctcgctggatggtcacgacgctaagcaggtgtggacgacc |
| | gcgtcgacggtggacgaggcgctggcccaactcgcgatgaccgacacggcgccggccgcggcttctcgc |
| | gccagccgcgtcccgctgtccgggatggcgctaccggtcgtcagcgccaagacggtgcagctcaacgacg |
| | gcgggttggtgcgcacggtgcacttgccggcccccaatgtcgcggggctgctgagtgcggccggcgtgcc |
| | gctgttgcaaagcgaccacgtggtgcccgccgcgacggccccgatcgtcgaaggcatgcagatccaggtg |
| | acccgcaatcggatcaagaaggtcaccgagcggctgccgctgccgccgaacgcgcgtcgtgtcgaggacc |
| | cggagatgaacatgagccgggaggtcgtcgaagacccgggggttccggggacccaggatgtgacgttcgc |
| | ggtagctgaggtcaacggcatcgagaccggccgtttgcccgtcgccaacgtcgtggtgaccccggcccacg |
| | aagccgtggtgcgggtgggcaccaagcccggtaccgaggtgcccccggtgatcgacggaagcatctggg |
| | acgcgatcgccgactgtgaggccggtggcaactgggcgatcaacaccggcaacaaatattacggtgatgtg |
| | cagatgaccagggcacctgggaggccaacggcgggctgcggtatgcaccccgcgctgacctcgccaccc |
| | gcgaagagcagatcaccgttgccgaggtgacccgactgcgtcaaggttggggcgcctgaccggtatatgct |
| | gcacgagcgggtgcgcgctga |
| | (SEQ ID NO: 73) |
| | MDFALLPPEYNSARMYTGPGAGSLLAAAGGWDSLAAELATTAEAY |
| | GSVLSGLAALHWRGPAAESMAVTAAPYIGWLYTTAEKTQQTAIQAR |
| | AAALAFEQAYAMTLPPPVVAANRIQLLALIATNFFGQNTAAIAATEA |
| | QYAEMWAQDAAAMYGYATASAAAALLTPFSPPRQTTNPAGLTAQA |
| | AAVSQATDPLSLLIETVTQALQALTIPSFIPEDFTFLDAIFAGYATVGV |
| | TQDVESFVAGTIGAESNLGLLNVGDENPAEVTPGDFGIGELVSATSPG |
| | GGVSASGAGGAASVGNTVLASVGRANSIGQLSVPPSWAAPSTRPVSA |
| | LSPAGLTTLPGTDVAEHGMPGVPGVPVAAGRASGVLPRYGVRLTVM |
| | AHPPAAGEFMIATTRDREGATMITFRLRLTCRTILRVFSRNPLVRGTD |
| | RLEAVVMLLAVTVSLLTIPFAAAAGTAVQDSRSHVYAHQAQTRHPA |
| | TATVIDHEGVIDSNTTATSAPPRTKITVPARWVVNGIERSGEVNAKPG |
| | TKSGDRVGIWVDSAGQLVDEPAPPARALNDAALAALGLWLSVAAVA |
| | GALLALTRAILIRVRNASWQHDIDSLFCTQRELMTTARDIMNAGVTC |
| | VGEHETLTAAAQYMREHDIGALPICGDDDRLHGMLTDRDIVIKGLAA |
| | GLDPNTATAGELARDSIYYVDANASIQEMLNVMEEHQVRRVPVISEH |
| | RLVGIVTEADIARHLPEHAIVQFVKAICSPMALASRQKGDTKFILNAK |
| | LACKTVTLTVDGTAMRVTTMKSRVIDIVEENGFSVDDRDDLYPAAG |
| | VQVHDADTIVLRRSRPLQISLDGHDAKQVWTTASTVDEALAQLAMT |
| | DTAPAAASRASRVPLSGMALPVVSAKTVQLNDGGLVRTVHLPAPNV |
| | AGLLSAAGVPLLQSDHVVPAATAPIVEGMQIQVTRNRIKKVTERLPLP |
| | PNARRVEDPEMNMSREVVEDPGVPGTQDVTFAVAEVNGVETGRLPV |
| | ANVVVTPAHEAVVRVGTKPGTEVPPVIDGSIWDAIAGCEAGGNWAI |
| | NTGNGYYGGVQFDQGTWEANGGLRYAPRADLATREEQIAVAEVTR |
| | LRQGWGAWPVCAARAGAR |
| | (SEQ ID NO: 74) |

Any Mtb antigen, including any Mtb antigen within any of the fusion proteins described herein, can have an amino acid sequence that is 100%, or from 70% to 99.9%, identical to the particular amino acid sequence listed in Tables 1-4. The amino acid sequence of any individual Mtb antigen, including any Mtb antigen within any of the fusion proteins described herein, can be at least 70%, at least 75%, at least 80%, at least 85%, at least 90%, at least 95%, at least 96%, at least 97%, at least 98%, or at least 99% identical to the particular amino acid sequence listed in Tables 1-4. Identity or similarity with respect to an amino acid or nucleotide sequence is defined herein as the percentage of amino acid residues (or nucleotide residues as the case may be) in the particular Mtb antigen that are identical (i.e., same residue) with the amino acid or nucleotide sequence for the Mtb antigen shown in Tables 1-4, after aligning the sequences and introducing gaps, if necessary, to achieve the maximum percent sequence identity. Percent sequence identity can be determined by, for example, the Gap program (Wisconsin Sequence Analysis Package, Version 8 for Unix,

Genetics Computer Group, University Research Park, Madison WI), using default settings, which uses the algorithm of Smith and Waterman (Adv. Appl. Math., 1981, 2, 482-489). Any amino acid number calculated as a % identity can be rounded up or down, as the case may be, to the closest whole number.

Any Mtb antigen, including any Mtb antigen within any of the fusion proteins described herein, can be fragments of the particular amino acid sequence listed in Tables 1-3. The amino acid sequence of any individual Mtb antigen, including any Mtb antigen within any of the fusion proteins described herein, can be missing consecutive amino acids constituting at least 20%, at least 15%, at least 10%, at least 5%, at least 4%, at least 3%, at least 2%, or at least 1%, of the particular amino acid sequence listed in Tables 1-3. The omitted consecutive amino acids may be from the C-terminus or N-terminus portion of the antigen. Alternately, the omitted consecutive amino acids may be from the internal portion of the antigen, thus retaining at least its C-terminus and N-terminus amino acids of the antigen.

Any Mtb antigen, including any Mtb antigen within any of the fusion proteins described herein, can have one or more amino acid additions, deletions, or substitutions compared to the particular amino acid sequence listed in Tables 1-3. Any individual Mtb antigen, including any Mtb antigen within any of the fusion proteins described herein, can have at least one, at least two, at least three, at least four, at least five, at least six, at least seven, at least eight, at least nine, at least ten, at least eleven, or at least twelve amino acid additions, deletions, or substitutions compared to the particular amino acid sequence listed in Tables 1-3. The amino acid additions, deletions, or substitutions can take place at any amino acid position within the Mtb antigen.

Where a particular Mtb antigen, including any Mtb antigen within any of the fusion proteins described herein, comprises at least one or more substitutions, the substituted amino acid(s) can each be, independently, any naturally occurring amino acid or any non-naturally occurring amino acid. Thus, a particular Mtb antigen may comprise one or more amino acid substitutions that are naturally occurring amino acids and/or one or more amino acid substitutions that are non-naturally occurring amino acids. Individual amino acid substitutions are selected from any one of the following: 1) the set of amino acids with nonpolar sidechains, for example, Ala, Cys, Ile, Leu, Met, Phe, Pro, Val; 2) the set of amino acids with negatively charged side chains, for example, Asp, Glu; 3) the set of amino acids with positively charged sidechains, for example, Arg, His, Lys; and 4) the set of amino acids with uncharged polar sidechains, for example. Asn, Cys, Gln, Gly, His, Met, Phe, Ser, Thr, Trp, Tyr, to which are added Cys, Gly, Met and Phe. Substitutions of a member of one , with another member of the same class are contemplated herein. Naturally occurring amino acids include, for example, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val), Non-naturally occurring amino acids include, for example, norleucine, omithine, norvaline, homoserine, and other amino acid residue analogues such as those described in Ellman et al., Meth. Enzym., 1991, 202, 301-336. To generate such non-naturally occurring amino acid residues, the procedures of Noren et al., Science, 1989, 244, 182 and Ellman et al., supra, can be used. Briefly, these procedures involve chemically activating a suppressor tRNA with a non-naturally occurring amino acid residue followed by in vitro transcription and translation of the RNA.

The Mtb antigens, including any Mtb antigen within any of the fusion proteins described herein, which are modified as described herein retain their ability to elicit an immune response against Mycobacterium tuberculosis. That is, modification of a particular Mtb antigen, including any Mtb antigen within any of the fusion proteins described herein, will still allow the resultant Mtb antigen, or fusion protein comprising the same, to elicit an immune response against Mycobacterium tuberculosis.

The present disclosure also provides nucleic acid molecules encoding any of the fusion proteins described herein that comprise at least three Mycobacterium tuberculosis (Mtb) antigens. In some embodiments, the fusion protein comprises at least one acute Mtb antigen, at least one latent Mtb antigen, and at least one resuscitation Mtb antigen. In some embodiments, the fusion protein comprises at least two latent Mtb antigens and at least one resuscitation Mtb antigen.

The nucleic acid molecules described herein and in Tables 1-4 are representative. That is, the specific sequences recited in Tables 1-4 are simply one example of a nucleic acid molecule that can encode a particular Mtb antigen within a fusion protein. One skilled in the art having knowledge of the genetic code can routinely prepare and design a plethora of nucleic acid molecules encoding the same Mtb antigen. The length and nucleotide content of any particular nucleic acid molecule is dictated by the desired amino acid sequence of the encoded Mtb antigen. The nucleic acid molecule sequences shown in Tables 1-4 are DNA, although RNA nucleic acid molecules are also contemplated.

TABLE 5

| Primer name | Sequence |
|---|---|
| 85B For NdeI | ata gat cat ata ttt agc cgt cct ggc ctg c (SEQ ID NO: 75) |
| 85B Rev EcoRI nostop | tta aga gaa ttc gcc cgc acc cag aga gga t (SEQ ID NO: 76) |
| ESAT-6 For BamHI | aac gtt gga tcc atg aca gag cag cag tgg aa SEQ ID NO: 77) |
| ESAT-6 Rev EcoRI ns | ata cta gaa ttc tgc gaa cat ccc agt gac gt (SEQ ID NO: 78) |
| 1733 For EcoRI | aac tta gaa ttc atg att gcg act acc cgt gat (SEQ ID NO: 79) |
| 1733 In1 Rev Xma | gat ata ccc ggg ggc ctc cag acg gtc ggt (SEQ ID NO: 80) |
| 1733 Out For Xma | aac gaa ccc ggg atc caa gac agc cgt agc c (SEQ ID NO: 81) |
| 1733 Out Rev Xba | taa gta tct aga atc ggc gat cgc acg cgc t (SEQ ID NO: 82) |
| 1733 In2 For Xba | ata gaa tct aga cgc gca att ctg atc cgc gt (SEQ ID NO: 83) |
| 1733 Rev ns SacI | aga taa gag ctc acg ttg ggt gca aaa cag gc (SEQ ID NO: 84) |
| 2626 For SacI | ata gaa gag ctc atg acc acg gcg cgt gat a (SEQ ID NO: 85) |
| 2626 Rev HindIII ns | taa aga aag ctt tgc att tag aat aaa ttt tgt gtc (SEQ ID NO: 86) |
| RpfD For HindIII | taa cta aag ctt ttg ctg ggc ctg agc acc (SEQ ID NO: 87) |
| RpfD Rev XhoI stop | atc taa ctc gag cta gtc atc gcg gct acc gct (SEQ ID NO: 88) |
| ESAT6 For NdeI | taa gat cat atg aca gag cag cag tgg aat ttc (SEQ ID NO: 89) |
| ESAT-6 Rev EcoRI ns | ata cta gaa ttc tgc gaa cat ccc agt gac gt (SEQ ID NO: 90) |

The present disclosure also provides vectors encoding any of the Mtb antigens, including Mtb antigens within any of the fusion proteins described herein, including any of the modified versions described herein. The vector can be capable of expressing an Mtb antigen in the cell of a mammal in a quantity effective to elicit an immune response in the mammal. The vector can be recombinant. The vector can comprise heterologous nucleic acid encoding the antigen. The vector can be a plasmid. The vector can be useful for transfecting cells with nucleic acid encoding an Mtb antigen, which the transformed host cell is cultured and maintained under conditions wherein expression of the antigen takes place.

In some embodiments, coding sequences can be optimized for stability and high levels of expression. In some instances, codons are selected to reduce secondary structure formation of the RNA such as that formed due to intramolecular bonding.

In some embodiments, the vectors can comprise regulatory elements for gene expression of the coding sequences of the nucleic acid. The regulatory elements can be a promoter, an enhancer an initiation codon, a stop codon, or a polyadenylation signal. In some embodiments, the vector can comprise heterologous nucleic acid encoding an Mtb antigen and can further comprise an initiation codon, which is upstream of the antigen coding sequence, and a stop codon, which is downstream of the antigen coding sequence. The initiation and termination codon are in frame with the antigen coding sequence.

The vector can also comprise a promoter that is operably linked to the antigen coding sequence. The promoter operably linked to the Mtb antigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter, or the like. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Representative examples of promoters include the bacteriophage T7 promoter, bacteriophage T3 promoter, SP6 promoter, lac operator-promoter, tac promoter, mycobacterial Hsp60 promoter, SV40 late promoter, SV40 early promoter, RSV-LTR promoter, CMV IE promoter, SV40 early promoter or SV40 late promoter and the CMV IE promoter.

The vector can also comprise a polyadenylation signal, which can be downstream of the antigen coding sequence. The polyadenylation signal can be a SV40 polyadenylation signal, LTR polyadenylation signal, CMV polyadeylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, CA).

The vector can also comprise an enhancer upstream of the consensus BoNT-A. BONT-B, BONT-E, and BoNT-F antigen sequences. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. The vector can be pVAX1, pCEP4 or pREP4 from Invitrogen (San Diego, CA), which can comprise the Epstein Barr virus origin of replication and nuclear antigen EBNA-1 coding region, which can produce high copy episomal replication without integration. The vector can be pVAX1or a pVax1variant with changes such as the variant plasmid described herein. The variant pVax1plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

The vector can also comprise a regulatory sequence, which can be well suited for gene expression in a mammalian or human cell into which the vector is administered. The consensus coding sequence can comprise a codon, which can allow more efficient transcription of the coding sequence in the host cell.

The vector can be pSE420 (Invitrogen, San Diego, Calif.) or pET28b (EMD Millipore, Billerca, Mass.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which may be used for protein production in mammalian cells such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989), which is incorporated fully by reference.

In some embodiments, the vector is a viral vector. Suitable viral vectors include, but are not limited to, an adenovirus vector, vaccinia virus vector, and paramyxovirus vector. Suitable adenovirus vectors include, but are not limited to, adenovirus 4, adenovirus 5, chimpanzee adenovirus 3, chimpanzee adenovirus 63, and chimpanzee adenovirus 68. A suitable vaccinia virus vector includes, but is not limited to, modified vaccinia Ankara (MVA). Suitable paramyxovirus vectors include, but are not limited to, modified parainfluenza virus (PIV2) and recombinant human parainfluenza virus (rHPIV2). In some embodiments, the vector is present within a composition comprising a pharmaceutically acceptable carrier. One skilled in the art is readily familiar with numerous vectors, many of which are commercially available.

The present disclosure also provides host cells comprising any of the nucleic acid molecules or vectors disclosed herein. The host cells can be used, for example, to express the Mtb antigens, or fragments of thereof. The Mtb antigens, or fragments thereof, can also be expressed in cells in vivo. The host cell that is transformed (for example, transfected) to produce the Mtb antigens, or fragments of thereof can be an immortalised mammalian cell line, such as those of lymphoid origin (for example, a myeloma, hybridoma, trioma or quadroma cell line). The host cell can also include normal lymphoid cells, such as B-cells, that have been immortalized by transformation with a virus (for example, the Epstein-Barr virus).

In some embodiments, the host cells include, but are not limited to: bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda (for example, Sf9) and Sf21 cell lines, and expresSF™ cells (Protein Sciences Corp., Meriden, CT, USA)), Drosophila S2 cells, and Trichoplusia in High FiveR Cells (Invitrogen, Carlsbad, CA, USA); and mammalian cells, such as COSI and COS7 cells, Chinese hamster ovary (CHO) cells, NSO myeloma cells, NIH 3T3 cells, 293 cells, Procell92S, perC6, HEPG2 cells, HeLa cells, L cells, HeLa, MDCK, HEK293, WI38, murine ES cell lines (for example, from strains 129/SV, C57/BL6, DBA-1, 129/SVJ), K562, Jurkat cells, and BW5147. Other useful mammalian cell lines are well known and readily available from the American Type Culture Collection (“ATCC”) (Manassas, VA, USA) and the National Institute of General Medical Sciences (NIGMS) Human Genetic Cell Repository at the Coriell Cell Repositories (Camden, NJ, USA). In some embodiments, the cell is a recombinant BCG. These cell types are only representative and are not meant to be an exhaustive list.

Among other considerations, some of which are described above, a host cell strain may be chosen for its ability to process the expressed Mtb antigens, or fragment thereof, in the desired fashion. Post-translational modifications of the polypeptide include, but are not limited to, glycosylation, acetylation, carboxylation, phosphorylation, lipidation, and acylation, and it is an aspect of the present disclosure to provide Mtb antigens thereof with one or more of these post-translational modifications.

In some embodiments, the recombinant BCG has been genetically engineered to express a functional endosomalytic protein that is bioactive at pH values near neutrality (e.g. about pH 6-8 or about 6.5 to 7.5). The endosomalytic protein is active within Mycobacteria-containing endosomes, which typically have an internal pH near neutrality. The activity of the endosomalytic protein produced by the rBCG results in disruption of the endosome, permitting the rBCG to escape from the endosome and into the cytoplasm of the cell. In some embodiments, the endosomalytic protein that is introduced into the rBCG by genetic engineering is Perfringolysin O (PfoA) from Clostridium perfringens or a mutant thereof, such as PfoAG1370, as described in WO 2007/058663, which is incorporated herein by reference in its entirety.

In some embodiments, the Mycobacteria are attenuated, as exemplified by BCG. However, those of skill in the art will recognize that other attenuated and nonattenuated Mycobacteria exist which would also be suitable for use herein. Examples of additional types of Mycobacteria include, but are not limited to, M. tuberculosis strain CDC1551, M. tuberculosis strain Beijing, M. tuberculosis strain H37Ra (ATCC #: 25177), M. tuberculosis strain H37Rv (ATCC #:25618), M. bovis (ATCC #: 19211 and 27291), M. fortuitum (ATCC #: 15073), M. smegmatis (ATCC #: 12051 and 12549), M. intracellulare (ATCC #: 35772 and 13209), M. kansasii (ATCC #: 21982 and 35775) M. avium (ATCC #: 19421 and 25291), M. gallinarum (ATCC #: 19711), M. vaccae (ATCC #: 15483 and 23024), M. leprae (ATCC #:), M. marinarum (ATCC #: 11566 and 11567), and M. microtti (ATCC #: 11152).

Examples of attenuated Mycobacterium strains include, but are not restricted To, M. tuberculosis pantothenate auxotroph strain, M. tuberculosis rpoV mutant strain, M. tuberculosis leucine auxotroph strain, BCG Danish strain (ATCC # 35733), BCG Japanese strain (ATCC # 35737), BCG Chicago strain (ATCC # 27289), BCG Copenhagen strain (ATCC #: 27290), BCG Pasteur strain (ATCC #: 35734), BCG Glaxo strain (ATCC #: 35741), BCG Connaught strain (ATCC # 35745), BCG Montreal (ATCC # 35746), BCG1331 strain, BCG Tokyo strain, BCG Moreau strain, BCG-Pasteur Aeras, and BCG Moscow strain.

In some embodiments, the cell comprising the one or more vector(s) is present within a composition comprising a pharmaceutically acceptable carrier.

In some embodiments, the Mtb antigen, or fragment thereof, is labeled with a detectable marker. Detectable markers include, but are not limited to, radioactive isotopes (such as P³² and S³⁵), enzymes (such as horseradish peroxidase, chloramphenicol acetyltransferase (CAT), β-galactosidase (β-gal), and the like), fluorochromes, chromophores, colloidal gold, dyes, and biotin. The labeled Mtb antigens, or fragments thereof, can be used to carry out diagnostic procedures in a variety of cell or tissue types. For imaging procedures, in vitro or in vivo, the Mtb antigens can be labeled with additional agents, such as NMR contrasting agents, X-ray contrasting agents, or quantum dots. Methods for attaching a detectable agent to polypeptides are known in the art. The Mtb antigens can also be attached to an insoluble support (such as a bead, a glass or plastic slide, or the like).

In some embodiments, the Mtb antigens, or fragment thereof, can be conjugated to a therapeutic agent including, but not limited to, radioisotopes (such as ¹¹¹In or ⁹⁰Y), toxins (such as tetanus toxoid or ricin), toxoids, and chemotherapeutic agents.

In some embodiments, the Mtb antigens, or fragments thereof, can be conjugated to an imaging agent. Imaging agents include, for example, a labeling moiety (such as biotin, fluorescent moieties, radioactive moieties, histidine tag or other peptide tags) for easy isolation or detection.

The present disclosure also provides compositions comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen. In some embodiments, the at least three Mtb antigens are not present in a fusion protein. In some embodiments, the at least three Mtb antigens are in the form of a protein and not nucleic acid molecules encoding the Mtb antigens.

In some embodiments, the acute Mtb antigen is Ag85B, ESAT6, MPT64, PPE15, PPE51, or Rv3615c. In some embodiments, the latent Mtb antigen is Rv1733c, Rv2626c. Rv3407, or Rv2628c. In some embodiments, the first and/or second transmembrane region of Rv1733c is deleted (Rv1733cΔTM). In some embodiments, the resuscitation Mtb antigen is RpfB, RpfD, or RpfE. In some embodiments, the composition comprises at least four Mtb antigens. In some embodiments, the composition comprises: ESAT6, Rv1733c, Rv2626c, and RpfD Mtb antigens; ESAT6, Rv1733c, Rv2626c, and RpfB Mtb antigens; RpfB, ESAT6, Rv1733c, and Rv2626c Mtb antigens; Ag85B, ESAT6, Rv1733c, Rv2626c, and RpfD Mtb antigens; Ag85B, ESAT6, Rv1733c, Rv2626c, and RpfB Mtb antigens; PPE51, Rv1733c, Rv2628c, and RpfD Mtb antigens; PPE51, Rv1733c, Rv2628c, and RpfB Mtb antigens; Rv3407, Rv1733c, Rv2626c, and RpfB Mtb antigens; or Rv3407, Rv1733c, Rv2626c, and RpfD Mtb antigens. In some embodiments, the composition further comprises a pharmaceutically acceptable carrier.

The present disclosure also provides compositions comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; wherein the composition comprises at least one nucleic acid molecule encoding at least one of the Mtb antigens. In some embodiments, the composition comprises one Mtb antigen in protein form and one or two nucleic acid molecules encoding two Mtb antigens. In some embodiments, the composition comprises two Mtb antigens in protein form, optionally as a fusion protein, and one nucleic acid molecule encoding one Mtb antigen. Thus, the present composition is a mixture of a protein Mtb antigen(s) and nucleic acid molecule(s) encoding an Mtb antigen(s).

In some embodiments, at least two Mtb antigens are encoded by one or more nucleic acid molecules within one or more vectors. In some embodiments, the one or more vectors is one or more viral vectors. In some embodiments. the one or more viral vectors are any one or more of adenovirus vector, vaccinia virus vector, or paramyxovirus vector. In some embodiments, the adenovirus vector is adenovirus 4, adenovirus 5, chimpanzee adenovirus 3, chimpanzee adenovirus 63, or chimpanzee adenovirus 68. In some embodiments, the one or more vaccinia virus vector is modified vaccinia Ankara (MVA). In some embodiments, the one or more paramyxovirus vectors are any one or more of modified parainfluenza virus (PIV2 or PIV3) or recombinant human parainfluenza virus (rHPIV2). In some embodiments, the at least two Mtb antigens are encoded by a single nucleic acid molecule within the same expression vector as a fusion protein.

In some embodiments, the acute Mtb antigen is Ag85B, ESAT6, MPT64, PPE15, PPE51, or Rv3615c. In some embodiments, the latent Mtb antigen is Rv1733c, Rv2626c, Rv3407, or Rv2628c. In some embodiments, the first and/or second transmembrane region of Rv1733c is deleted. In some embodiments, the resuscitation Mtb antigen is RpfB, RpfD, or RpfE. In some embodiments, the composition comprises at least four Mtb antigens. In some embodiments, the composition comprises: ESAT6, Rv1733c, Rv2626c, and RpfD Mtb antigens; ESAT6, Rv1733c, Rv2626c, and RpfB Mtb antigens; RpfB, ESAT6, Rv1733c, and Rv2626c Mtb antigens; Ag85B, ESAT6, Rv1733c, Rv2626c, and RpfD Mtb antigens; Ag85B, ESAT6, Rv1733c, Rv2626c, and RpfB Mtb antigens; PPE51, Rv1733c, Rv2628c, and RpfD Mtb antigens; PPE51, Rv1733c, Rv2628c, and RpfB Mtb antigens; Rv3407. Rv1733c, Rv2626c, and RpfB Mtb antigens; or Rv3407, Rv1733c, Rv2626c, and RpfD Mtb antigens; wherein any two or more Mtb antigens can be within a fusion protein. In some embodiments, the composition comprises at least four Mtb antigens. In some embodiments. the composition further comprises a pharmaceutically acceptable carrier.

In some embodiments, where a rBCG is used as the vehicle to deliver the Mtb antigens, or fusion proteins, or nucleic acids and or vectors comprising or encoding the same. expression of all or part of the Dos R regulon is not up-regulated in the rBCG. In some embodiments, one or more of the following Dos R regulon antigens are not up-regulated in the rBCG: Rv1738, Rv2623, Rv2031c, Rv2032, Rv2626c, Rv2005c, Rv3127, Rv1733c, Rv1996, Ry2628c, Rv0079, Rv3130c, Rv3131, Rv1813c, Rv2006, Rv2029c, Rv2627c, Rv2030c, Rv3132c, and Rv2629. In some embodiments, the rBCG does not comprise up-regulation of: 1) one or more Mtb antigens, including “classical” Mtb antigens such as 85A, 85B and TB 10.4; and 2) at least one Mtb resuscitation antigen selected from Rv0867c, Rv1009, Rv1884c, Rv2389c, Rv2450c, Rv0288, Rv1009, Rv0685, Rv0824c, Rv1349, Rv2744c, Rv3347c, Rv1130, and Rv1169c. In some embodiments, the rBCG does not include the expression of the following combinations; classical antigens Rv1886c, Rv3804c; resuscitation antigens Rv0867c, Rv1884c, Rv2389c; and Mtb-specific antigen Rv3407. In some embodiments, the rBCG does not include the expression of the following combination: Rv3804c, Rv1886c, and Rv3407, or in addition with Rv3133c, and with the combination of Rv0867c, Rv1884c, and Rv2389c. In some embodiments, the rBCG does not include the expression of the following combination; TB10.4, Ag85B, Ag85A, and Rv3407. In some embodiments, the cell is not a rBCG.

The present disclosure also provides compositions comprising any one or more of the fusion proteins. Mtb antigens, nucleic acid molecules encoding Mtb antigens, including fusion proteins thereof, cells, and/or vectors and a pharmaceutically acceptable carrier. Compositions include, for example, pharmaceutical compositions. A pharmaceutically acceptable carrier refers to at least one component of a pharmaceutical preparation that is normally used for administration of active ingredients. As such, a carrier can contain any pharmaceutical excipient used in the art and any form of vehicle for administration. Carriers include, but are not limited to, phosphate buffered saline. physiological saline, water, citrate/sucrose/Tween formulations and emulsions such as, for example, oil/water emulsions.

The compositions can also include an active therapeutic agent and a variety of other pharmaceutically acceptable components. See Remington's Pharmaceutical Science (15th ed., Mack Publishing Company, Easton, Pennsylvania (1980))). The desired form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents include, but are not limited to, distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica. Additional excipients include, for example, colorants, taste-masking agents, solubility aids, suspension agents, compressing agents, enteric coatings, sustained release aids, and the like.

In some embodiments, the compositions can be administered in the form of a depot injection or implant preparation, which can be formulated in such a manner as to permit a sustained release. An exemplary composition comprises any one or more of the compositions described herein formulated in aqueous buffer.

In some embodiments, liquid formulations of a pharmaceutical composition for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. Liquid formulations of pharmaceutical compositions can also include solutions, emulsions, syrups and elixirs containing, together with the active compound(s), wetting agents, sweeteners, and coloring and flavoring agents. Various liquid and powder formulations of the pharmaceutical compositions can be prepared by conventional methods for inhalation into the lungs of the mammal to be treated.

In some embodiments, liquid formulations of a pharmaceutical composition for injection can comprise various carriers such as vegetable oils, dimethylacetamide, dimethylformamide, ethyl lactate, ethyl carbonate, isopropyl myristate, ethanol, polyols such as, for example, glycerol, propylene glycol, liquid polyethylene glycol, and the like. In some embodiments, the composition includes a citrate/sucrose/tween carrier. For intravenous injections, water soluble versions of the compositions can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution or other suitable excipients. A suitable insoluble form of the composition can be prepared and administered as a suspension in an aqueous base or a pharmaceutically acceptable oil base, such as an ester of a long chain fatty acid such as, for example, ethyl oleate.

The compositions can be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. In some embodiments, for topical applications, the pharmaceutical compositions can be formulated in a suitable ointment. In some embodiments, a topical semi-solid ointment formulation typically comprises a concentration of the active ingredient from about 1 to 20%, or from 5 to 10%, in a carrier, such as a pharmaceutical cream base. Some examples of formulations of a composition for topical use include, but are not limited to, drops, tinctures, lotions, creams, solutions, and ointments containing the active ingredient and various supports and vehicles.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or microparticles such as polylactide, polyglycolide, or copolymer for enhanced adjuvant effect (see Langer, Science, 1990, 249, 1527 and Hanes, Advanced Drug Delivery Reviews, 1997, 28, 97). A sterile injectable preparation such as, for example, a sterile injectable aqueous or oleaginous suspension can also be prepared. This suspension may be formulated according to techniques known in the art using suitable dispersing, wetting, and suspending agents. In some embodiments, the pharmaceutical composition can be delivered in a microencapsulation device so as to reduce or prevent a host immune response against the protein.

Effective doses of the compositions of the present disclosure, for the treatment of a condition vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

In some embodiments, the compositions can be administered to a subject by injection intravenously, subcutaneously, intraperitoneally, intramuscularly, intramedullarily, intraventricularly, intraepidurally, intraarterially, intravascularly, intraarticularly, intrasynovially, intrasternally, intrathecally, intrahepatically, intraspinally, intratumorly, intracranially, enteral, intrapulmonary, transmucosal, intrauterine, sublingual, or locally at sites of inflammation or tumor growth by using standard methods. Alternately, the compositions can be administered to a subject by routes including oral, nasal, ophthalmic, rectal, or topical. The most typical route of administration is intravascular, subcutaneous, or intramuscular, although other routes can be effective. In some embodiments, compositions are administered as a sustained release composition or device, such as a Medipad™ device. The composition can also be administered via the respiratory tract, for example, using a dry powder inhalation device, nebulizer, or a metered dose inhaler. The composition can also be administered by traditional syringes, needleless injection devices, “microprojectile bombardment gone guns,” or other physical methods such as electroporation (“EP”), “hydrodynamic method”, or ultrasound.

In some embodiments, the composition can be administered to a subject by sustained release administration, by such means as depot injections of erodible implants directly applied during surgery or by implantation of an infusion pump or a biocompatible sustained release implant into the subject. Alternately, the composition can be administered to a subject by injectable depot routes of administration, such as by using 1-, 3-, or 6-month depot injectable or biodegradable materials and methods, or by applying to the skin of the subject a transdermal patch containing the composition, and leaving the patch in contact with the subject's skin, generally for 1 to 5 hours per patch.

In some embodiments, the compositions comprise about 1 nanogram to about 10 mg of nucleic acid. In some embodiments, the compositions comprise: 1) at least 10, 15, 20, 25, 30, 35, 40, 45, 50, 55, 60, 65, 70, 75, 80, 85, 90, 95 or 100 nanograms, or at least 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg.

In some embodiments, the compositions comprise about 5 nanograms to about 10 mg of nucleic acid molecule. In some embodiments, the compositions comprise about 25 nanograms to about 5 mg of nucleic acid molecule. In some embodiments, the compositions contain about 50 nanograms to about 1 mg of nucleic acid molecule. In some embodiments, the compositions contain about 0.1 to about 500 micrograms of nucleic acid molecule. In some embodiments, the compositions contain about 1 to about 350 micrograms of nucleic acid molecule. In some embodiments, the compositions contain about 5 to about 250 micrograms of nucleic acid molecule. In some embodiments, the compositions contain about 10 to about 200 micrograms of nucleic acid molecule. In some embodiments, the compositions contain about 15 to about 150 micrograms of nucleic acid molecule. In some embodiments, the compositions contain about 20 to about 100 micrograms of nucleic acid molecule. In some embodiments, the compositions contain about 25 to about 75 micrograms of nucleic acid molecule. In some embodiments, the compositions contain about 30 to about 50 micrograms of nucleic acid molecule. In some embodiments, the compositions contain about 35 to about 40 micrograms of nucleic acid molecule. In some embodiments, the compositions contain about 100 to about 200 micrograms of nucleic acid molecule. In some embodiments, the compositions comprise about 10 to about 100 micrograms of nucleic acid molecule. In some embodiments, the compositions comprise about 20 to about 80 micrograms of nucleic acid molecule. In some embodiments, the compositions comprise about 25 to about 60 micrograms of nucleic acid molecule. In some embodiments, the compositions comprise about 30 nanograms to about 50 micrograms of nucleic acid molecule. In some embodiments, the compositions comprise about 35 nanograms to about 45 micrograms of nucleic acid molecule. In some embodiments, the compositions contain about 0.1 to about 500 micrograms of nucleic acid molecule. In some embodiments, the compositions contain about 1 to about 350 micrograms of nucleic acid molecule. In some embodiments, the compositions contain about 25 to about 250 micrograms of nucleic acid molecule. In some embodiments, the compositions contain about 100 to about 200 micrograms of nucleic acid molecule.

The compositions can be formulated according to the mode of administration to be used. In cases where compositions are injectable pharmaceutical compositions, they are sterile, pyrogen free and particulate free. An isotonic formulation can be used. Generally, additives for isotonicity can include sodium chloride, dextrose, mannitol, sorbitol and lactose. In some cases, isotonic solutions such as phosphate buffered saline are suitable. Stabilizers include gelatin and albumin. In some embodiments, a vasoconstriction agent is added to the formulation.

The compositions can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freund's incomplete adjuvant, LPS analog including monophosphoryl lipid A, muramyl peptides, quinone analogs, vesicles such as squalene and squalane, hyaluronic acid, lipids, liposomes, calcium ions, viral proteins, polyanions. polycations, or nanoparticles, or other known transfection facilitating agents, The transfection facilitating agent is a polyanion, polycation, including poly-L-glutamate (LGS), or lipid. The transfection facilitating agent is poly-L-glutamate, and more suitably, the poly-L-glutamate is present in the composition at a concentration less than 6 mg/ml. The transfection facilitating agent can also include surface active agents such as immune-stimulating complexes (ISCOMS). Freunds incomplete adjuvant, LPS analog including monophosphoryl lipid A, muramyl peptides, quinone analogs and vesicles such as squalene and squalane, and hyaluronic acid can also be used administered in conjunction with the genetic construct. In some embodiments, the plasmid compositions can also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant may be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes which may be useful adjuvants include those encoding: MCP-1, MIP-1a, MIP-1p, IL-8, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFKB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The plasmid compositions can further comprise a genetic vaccine facilitator agent as described in U.S. Ser. No. 021,579 filed Apr. 1, 1994, which is fully incorporated by reference.

The present disclosure also provides kits comprising any of the Mtb antigens, fragments thereof, fusion proteins, nucleic acid molecules, vectors, or cells, described herein. The kit can include, for example, container(s), package(s) or dispenser(s) along with labels and instructions for administration or use.

The present disclosure also provides methods of eliciting an immune response against Mycobacterium tuberculosis in a mammal comprising administering to the mammal an immunologically sufficient amount of one or more fusion proteins comprising at least three Mycobacterium tuberculosis (Mtb) antigens, wherein at least one fusion protein comprises: at least one acute Mtb antigen, at least one latent Mtb antigen, and at least one resuscitation Mtb antigen; or at least two latent Mtb antigens, and at least one resuscitation Mtb antigen. Any of the fusion proteins described herein can be administered. In some embodiments, the fusion protein comprises at least four Mtb antigens.

The present disclosure also provides methods of eliciting an immune response against Mycobacterium tuberculosis in a mammal comprising administering to the mammal an immunologically sufficient amount of a composition comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier. Any of the compositions comprising three or more Mtb antigens can be administered. In some embodiments, the composition comprises at least four Mtb antigens.

The present disclosure also provides methods of eliciting an immune response against Mycobacterium tuberculosis in a mammal comprising administering to the mammal an immunologically sufficient amount of a composition comprising at least three Mycobacterium tuberculosis (Mtb) antigens, wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens. Any of the compositions comprising a mixture of one or more Mtb antigen proteins and one of more nucleic acid molecules encoding one or more Mtb antigens described herein can be administered.

The fusion proteins and compositions described herein can be used to treat or prevent tuberculosis. In some embodiments, the method comprises administering to a human a therapeutically- or prophylactically-effective amount of any of the fusion proteins or compositions described herein such that the tuberculosis infection is diminished or prevented.

In some embodiments, the subject being treated will have been previously diagnosed as having tuberculosis. Such subjects will, thus, have been diagnosed as being in need of such treatment. Alternately, the treatment may be intended to prevent a tuberculosis infection in a subject that does not yet have tuberculosis or to a subject that is travelling to an area where tuberculosis is prevalent.

Treatment of a subject suffering from tuberculosis can be monitored using standard methods. Some methods entail determining a baseline value, for example, of an antibody level or profile in a subject, before administering a dosage of agent, and comparing this with a value for the profile or level after treatment. A significant increase such as, for example, greater than the typical margin of experimental error in repeat measurements of the same sample, expressed as one standard deviation from the mean of such measurements in value of the level or profile signals a positive treatment outcome (i.e., that administration of the agent has achieved a desired response). If the value for immune response does not change significantly, or decreases, a negative treatment outcome is indicated.

In other embodiments, a control value such as a mean and standard deviation, of level or profile is determined for a control population. Typically the individuals in the control population have not received prior treatment. Measured values of the level or profile in a subject after administering a therapeutic agent are then compared with the control value. A significant increase relative to the control value, such as greater than one standard deviation from the mean, signals a positive or sufficient treatment outcome. A lack of significant increase or a decrease signals a negative or insufficient treatment outcome. Administration of the therapeutic is generally continued while the level is increasing relative to the control value. As before, attainment of a plateau relative to control values is an indicator that the administration of treatment can be discontinued or reduced in dosage and/or frequency.

In other embodiments, a control value of the level or profile, such as a mean and standard deviation, is determined from a control population of individuals who have undergone treatment with a therapeutic agent and whose levels or profiles have plateaued in response to treatment. Measured values of levels or profiles in a subject are compared with the control value. If the measured level in a subject is not significantly different, such as by more than one standard deviation, from the control value, treatment can be discontinued. If the level in a subject is significantly below the control value, continued administration of agent is warranted. If the level in the subject persists below the control value, then a change in treatment may be indicated.

In other embodiments, a subject who is not presently receiving treatment but has undergone a previous course of treatment is monitored for antibody levels or profiles to determine whether a resumption of treatment is required. The measured level or profile in the subject can be compared with a value previously achieved in the subject after a previous course of treatment. A significant decrease relative to the previous measurement, such as greater than a typical margin of error in repeat measurements of the same sample, is an indication that treatment can be resumed. Alternately, the value measured in a subject can be compared with a control value (mean plus standard deviation) determined in a population of subjects after undergoing a course of treatment. Alternately, the measured value in a subject can be compared with a control value in populations of prophylactically treated subjects who remain free of symptoms of disease, or populations of therapeutically treated subjects who show amelioration of disease characteristics. In all of these cases, a significant decrease relative to the control level, such as more than a standard deviation, is an indicator that treatment should be resumed in a subject.

In some methods, a baseline measurement of antibody to a given antigen in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline, such as 50%, 25% or 10%, administration of a further dosage of antigen is administered. In some embodiments, peak or subsequent measured levels less background are compared with reference levels previously determined to constitute a beneficial prophylactic or therapeutic treatment regime in other subjects. If the measured antibody level is significantly less than a reference level, such as less than the mean minus one standard deviation of the reference value in population of subjects benefiting from treatment, administration of an additional dosage of antigen is indicated.

In some embodiments, the subject(s) that can be treated by the above-described methods is an animal, such as a mammal, including, but are not limited to, humans, non-human primates, rodents (including rats, mice, hamsters and guinea pigs) cow, horse, sheep, goat, pig, dog and cat. In most instances, the mammal is a human.

The present disclosure also provides fusion proteins for use in the preparation of a medicament for treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides fusion proteins for use in treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides uses of a fusion protein in the preparation of a medicament for treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides uses of a fusion protein in treating or preventing a Mycobacterium tuberculosis infection, wherein the fusion protein comprises at least three Mycobacterium tuberculosis (Mtb) antigens, and wherein the fusion protein comprises: at least one acute Mtb antigen, at least one latent Mtb antigen, and at least one resuscitation Mtb antigen; or at least two latent Mtb antigens, and at least one resuscitation Mtb antigen.

The present disclosure also provides compositions for use in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides compositions for use in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides uses of a composition in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides uses of a composition in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier.

The present disclosure also provides compositions for use in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides compositions for use in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides uses of a composition in the preparation of a medicament for treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides uses of a composition in treating or preventing a Mycobacterium tuberculosis infection, wherein the composition comprises at least three Mycobacterium tuberculosis (Mtb) antigens, and wherein the composition comprises: at least one acute Mtb antigen, at least one latent Mtb antigen, and at least one resuscitation Mtb antigen; or at least two latent Mtb antigens, and at least one resuscitation Mtb antigen; and a pharmaceutically acceptable carrier; wherein the composition comprises at least one nucleic acid molecule encoding at least one of the Mtb antigens.

The present disclosure also provides any of the fusion proteins described herein, or any of the compositions described herein, or any of the cells described herein, or any of the vectors described herein, or any of the methods described herein, or any of the uses described herein, substantially as described with reference to the accompanying examples and/or figures.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Construction of the Antigen Cassette as the Basic Tool for Subsequent Platform Insertion

The 5 antigen cassette (Construct D), which was human codon optimized, was synthesized commercially by Aldevron and cloned into pVAX-1. For use in MVA vectors, antigen 85B was synthesized with its native leader sequence. For viral vectors other than MVA, antigen 85B was replaced with genes either containing or not containing the leader sequence, this being achieved using the unique EcoRI and XmaI nuclease target sequences. To clone the 5 antigen cassette into adenoviral or CMV vectors, primers with homology arms were used to PCR amplify the cassette, and this PCR product was recombined into the appropriate region of the BAC.

For recombinant protein expression of Construct D, the 85B, Rv1733, Rv2626, and RpfD genes were synthesized by DNA2.0 and codon optimized for E. coli expression. Antigen 85B and RpfD were synthesized without the native leader sequences. Each gene was PCR amplified from the respective DNA2.0 vector with appropriate restriction sites added and cloned into pET28b sequentially. ESAT-6 was PCR amplified from H37Rv DNA.

More specifically, the genes encoding the protein antigens were PCR amplified using the primers in Table 5 and cloned into the pET28b vector (Novagen) via the indicated restriction enzyme sites. ESAT6 was PCR amplified from Mtb and first cloned into the pET23b vector (Novagen). It was subsequently PCR amplified and cloned into pET28b. The genes for antigen 85B, Rv1733c, Rv2626c, and RpfD were all synthesized with their codons optimized for expression in E. coli (DNA2.0). Antigen Ag85B and rpfD were synthesized without the bases encoding the N-terminal signal sequence, and rpfB was PCR amplified from Mtb without the N-terminal signal sequence. The codon optimized genes were PCR amplified and cloned into pET28b creating N-terminal 6xHis-tagged fusion proteins. The genes were cloned with no spacer sequences, only the restriction enzyme sites between each gene. To remove the 2 transmembrane regions of Rv1733c, it was PCR amplified in 3 pieces which were ligated together.

In another embodiment the 4 Ag and 5 Ag proteins were constructed with wild type Rv1733c including the transmembrane regions. The pET28b constructs were cloned in E. coli cloning strains, screened by restriction digest and sequenced to verify each construct.

### Example 2: Construction of Recombinant BCG (rBCG) Strains

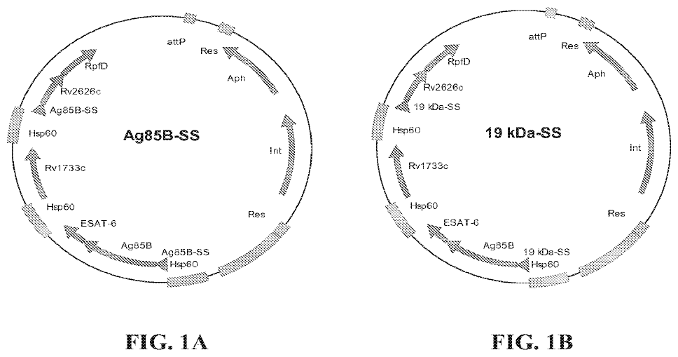

rBCG strains over-expressing antigens involved with active infection, latency, and resuscitation were constructed. The genes of interest were first cloned in a plasmid which allows for their insertion in the chromosome of BCG at the attB integration site (pJFINT-RIAR). Since this plasmid has three different cloning sites, the 5 genes were not fused together but rather split into three groups. Ag85B was fused to ESAT-6 (Ag85B-ESAT6; SEQ ID NO:91 (nucleotide) and SEQ ID NO:92 (amino acid) with Ag85B signal sequence; SEQ ID NO:93 (nucleotide) and SEQ ID NO:94 (amino acid) with 19 kDa lipoprotein signal sequence) and placed in the first cloning site, Rv1733c was cloned by itself, and Rv2626c was fused with RpfD (Rv2626c-RpfD; SEQ ID NO:95 and SEQ ID NO:96 with Ag85B signal sequence; SEQ ID NO:97 and SEQ ID NO:98 with 19 kDa lipoprotein signal sequence) then placed into the third site to create the following construct; Ag85B-ESAT6+Rv1733c+Rv2626c-RpfD. Each insert was placed under the control of the Hsp60 promoter and a signal sequence was added to both fusions, except Rv1733c since it is already a mycobacterial membrane protein. Constructs with two different signal sequences were made; the Ag85B signal sequence which allows for secretion of the fusions, and the 19 kDa lipoprotein signal sequence which anchors them into the membrane to examine which one would allow better expression and/or immunogenicity of the antigens. Both replicating and non-replicating versions of those rBCG strains were constructed. The maps of the plasmids that were used to construct the rBCG strains are shown in FIG. 1A (fusions with the Ag85B signal sequence) and 1B (fusions with the 19 kDa signal sequence).

After the rBCG strains were constructed by integration of the shuttle plasmids in the chromosome of BCG SSI. BCG SSIAPanCD or other BCG strains, cell lysates and supernatants were prepared to evaluate the relative expression of the different antigens as well as their localization in the rBCG cells. Western blot data using a monoclonal antibody against ESAT-6 as a probe showed the following: the BCG SSI control, which does not have the gene for ESAT-6, showed no reactivity with the monoclonal antibody in either the cell lysate or the culture supernatant; for rBCG expressing the Ag85B-ESAT6 fusion linked to the 19 kDa signal sequence, low levels of the fusion were detected, but only in the cell lysates and not in the culture supernatant of both the replicating and non-replicating rBCG strains, showing that this signal sequence does not result in secretion of the fusion, as expected; the rBCG strain expressing the Ag85B-ESAT6 fusion linked to the Ag85B signal sequence showed a very high level of expression both in the cell lysate and the culture supernatant confirming that this signal sequence does result in the secretion of this fusion (data not shown).

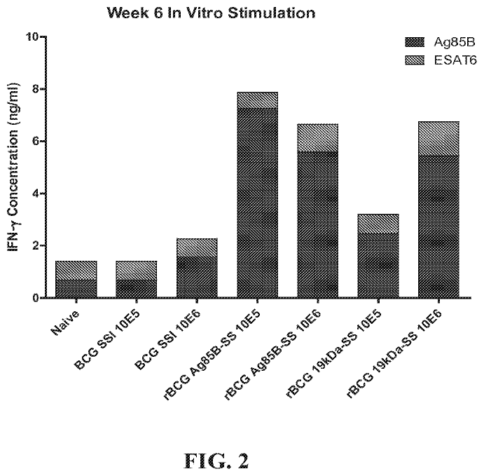

Following the antigen expression studies, a preliminary immunogenicity experiment was carried out in C57/BL6 mice comparing the BCG SSI control and the rBCG strains expressing the fusions with the two different signal sequences. Mice were immunized once sc with either 10⁵ or 10⁶ CFUs of the different BCG strains and were sacrificed 6 weeks later. The splenocytes were stimulated for 72 hours with recombinant Ag85B or ESAT-6 proteins and the levels of antigen specific IFNγ released in the culture supernatants were measured using an ELISA assay. The following results were obtained with the Ag85B-ESAT6 fusions (see, FIG. 2): the BCG SSI control given at the lowest dose showed background levels of IFNγ, similar to what was obtained with the naïve mice, whereas the mice given the highest dose showed higher levels of Ag85B specific IFNγ, but no ESAT6 specific response which was expected since the BCG SSI control does not have the gene for ESAT-6; in contrast, the rBCG strain expressing the Ag85B-ESAT6 fusion linked to the Ag85B signal sequence gave a much stronger Ag85B specific response at both doses, but an ESAT-6 response above background only at the higher dose; similar results were obtained with the rBCG strain expressing the fusion linked to the 19 kDa signal sequence, but only at the higher dose, which is not surprising considering the expression levels were much lower in that strain (data not shown).
DNA sequence of the Ag85B-ESAT6 fusion with the Ag85B signal sequence:

(SEQ ID NO: 91)
atgacagacgtgagccgaaagattcgagcttggggacgccgattgatga
tcggcacggcagcggctgtagtccttccgggcctggtggggcttgccgg
cggagcggcaaccgcgggcgcgttctcccggccggggctgccggtcgag
tacctgcaggtgccgtcgccgtcgatgggccgcgacatcaaggttcagt
tccagagcggtgggaacaactcacctgcggtttatctgctcgacggcct
gcgcgcccaagacgactacaacggctgggatatcaacaccccggcgttc
gagtggtactaccagtcgggactgtcgatagtcatgccggtcggcgggc
agtccagcttctacagcgactggtacagcccggcctgcggtaaggctgg
ctgccagacttacaagtgggaaaccttcctgaccagcgagctgccgcaa
tggttgtccgccaacagggccgtgaagcccaccggcagcgctgcaatcg
gcttgtcgatggccggctcgtcggcaatgatcttggccgcctaccaccc
ccagcagttcatctacgccggctcgctgtcggccctgctggacccctct
caggggatggggcctagcctgatcggcctcgcgatgggtgacgccggcg
gttacaaggccgcagacatgtggggtccctcgagtgacccggcatggga
gcgcaacgaccctacgcagcagatccccaagctggtcgcaaacaacacc
cggctatgggtttattgcgggaacggcaccccgaacgagttgggcggtg
ccaacatacccgccgagttcttggagaacttcgttcgtagcagcaacct
gaagttccaggatgcgtacaacgccgcgggcgggcacaacgccgtgttc
aacttcccgcccaacggcacgcacagctgggagtactggggcgctcagc
tcaacgccatgaagggtgacctgcagagttcgttaggcgccggcatgac
agagcagcagtggaatttcgcgggtatcgaggccgcggcaagcgcaatc
cagggaaatgtcacgtccattcattccctccttgacgaggggaagcagt
ccctgaccaagctcgcagcggcctggggcggtagcggttcggaggcgta
ccagggtgtccagcaaaaatgggacgccacggctaccgagctgaacaac
gcgctgcagaacctggcgcggacgatcagcgaagccggtcaggcaatgg
cttcgaccgaaggcaacgtcactgggatgttcgcatga.

Amino acid sequence of the Ag85B-ESAT6 fusion with the Ag85B signal sequence:

(SEQ ID NO: 92)
MTDVSRKIRAWGRRLMIGTAAAVVLPGLVGLAGGAATAGAFSRPGLPVE
YLQVPSPSMGRDIKVQFQSGGNNSPAVYLLDGLRAQDDYNGWDINTPAF
EWYYQSGLSIVMPVGGQSSFYSDWYSPACGKAGCQTYKWETFLTSELPQ
WLSANRAVKPTGSAAIGLSMAGSSAMILAAYHPQQFIYAGSLSALLDPS
QGMGPSLIGLAMGDAGGYKAADMWGPSSDPAWERNDPTQQIPKLVANNT
RLWVYCGNGTPNELGGANIPAEFLENFVRSSNLKFQDAYNAAGGHNAVF
NFPPNGTHSWEYWGAQLNAMKGDLQSSLGAGMTEQQWNFAGIEAAASAI
QGNVTSIHSLLDEGKQSLTKLAAAWGGSGSEAYQGVQQKWDATATELNN
ALQNLARTISEAGQAMASTEGNVTGMFA.

DNA sequence of the Ag85B-ESAT6 fusion with the 19 kDa signal sequence:

(SEQ ID NO: 93)
atgaagcgtggactgacggtcgcggtagccggagccgccattctggtcg
caggtctttccggatgttcaagcaacaagtcgactacaggaagcggtga
gaccacgaccgcggcaggtaccacggcaagccccggccggccggggctg
ccggtcgagtacctgcaggtgccgttcgccgtcgatgggccgcgacatc
aaggttcagttccagagcggtgggaacaactcacctgcggtttatctgc
tcgacggcctgcgcgcccaagacgactacaacggctgggatatcaacac
cccggcgttcgagtggtactaccagtcgggactgtcgatagtcatgccg
gtcggcgggcagtccagcttctacagcgactggtacagcccggcctgcg
gtaaggctggctgccagacttacaagtgggaaaccttcctgaccagcga
gctgccgcaatggttgtccgccaacagggccgtgaagcccaccggcagc
gctgcaatcggcttgtcgatggccggctcgtcggcaatgatcttggccg
cctaccacccccagcagttcatctacgccggctcgctgtcggccctgct
ggacccctctcaggggatggggcctagcctgatcggcctcgcgatgggt
gacgccggcggttacaaggccgcagacatgtggggtccctcgagtgacc
cggcatgggagcgcaacgaccctacgcagcagatccccaagctggtcgc
aaacaacacccggctatgggtttattgcgggaacggcaccccgaacgag
ttgggcggtgccaacatacccgccgagttcttggagaacttcgttcgta
gcagcaacctgaagttccaggatgcgtacaacgccgcgggcgggcacaa
cgccgtgttcaacttcccgcccaacggcacgcacagctgggagtactgg
ggcgctcagctcaacgccatgaagggtgacctgcagagttcgttaggcg
ccggcatgacagagcagcagtggaatttcgcgggtatcgaggccgcggc
aagcgcaatccagggaaatgtcacgtccattcattccctccttgacgag
gggaagcagtccctgaccaagctcgcagcggcctggggcggtagcggtt
cggaggcgtaccagggtgtccagcaaaaatgggacgccacggctaccga
gctgaacaacgcgctgcagaacctggcgcggacgatcagcgaagccggt
caggcaatggcttcgaccgaaggcaacgtcactgggatgttcgcatga.

Amino acid sequence of the Ag85B-ESAT6 fusion with the 19 kDa signal sequence:

(SEQ ID NO: 94)
MKRGLTVAVAGAAILVAGLSGCSSNKSTTGSGETTTAAGTTASPGRPGL
PVEYLQVPSPSMGRDIKNOFQSGGNNSPAVYLLDGLRAQDDYNGWDINT
PAFEWYYQSGLSIVMPVGGQSSFYSDWYSPACGKAGCQTYKWETFLTSE
LPQWLSANRAVKPTGSAAIGLSMAGSSAMILAAYHPQQFIYAGSLSALL
DPSQGMGPSLIGLAMGDAGGYKAADMWGPSSDPAWERNDPTQQIPKLVA
NNTRLWVYCGNGTPNELGGANIPAEFLENFVRSSNLKFQDAYNAAGGHN
AVFNFPPNGTHSWEYWGAQLNAMKGDLQSSLGAGMTEQQWNFAGIEAAA
SAIQGNVTSIHSLLDEGKQSLTKLAAAWGGSGSEAYQGVQQKWDATATE
LNNALQNLARTISEAGQAMASTEGNVTGMFA.

DNA sequence of Rv2626c-RpfD fusion with the Ag85B signal sequence:

(SEQ ID NO: 95)
atgacagacgtgagccgaaagattcgagcttggggacgccgattgatga
tcggcacggcagcggctgtagtccttccgggcctggtggggcttgccgg
cggagcggcaaccgcgggcgcgttctccatgaccaccgcacgcgacatc
atgaacgcaggtgtgacctgtgttggcgaacacgagacgctaaccgctg
ccgctcaatacatgcgtgagcacgacatcggcgcgttgccgatctgcgg
ggacgacgaccggctgcacggcatgctcaccgaccgcgacattgtgatc
aaaggcctggctgcgggcctagacccgaataccgccacggctggcgagt
tggcccgggacagcatctactacgtcgatgcgaacgcaagcatccagga
gatgctcaacgtcatggaagaacatcaggtccgccgtgttccggtcatc
tcagagcaccgcttggtcggaatcgtcaccgaagccgacatcgcccgac
acctgcccgagcacgccattgtgcagttcgtcaaggcaatctgctcgcc
catggccctcgccagcatgacaccgggtttgcttactactgcgggtgct
ggccgaccacgtgacaggtgcgccaggatcgtatgcacggtgttcatcg
aaaccgccgttgtcgcgaccatgtttgtcgcgttgttgggtctgtccac
catcagctcgaaagccgacgacatcgattgggacgccatcgcgcaatgc
gaatccggcggcaattgggcggccaacaccggtaacgggttatacggtg
gtctgcagatcagccaggcgacgtgggattccaacggtggtgtcgggtc
gccggcggccgcgagtccccagcaacagatcgaggtcgcagacaacatt
atgaaaacccaaggcccgggtgcgtggccgaaatgtagttcttgtagtc
agggagacgcaccgctgggctcgctcacccacatcctgacgttcctcgc
ggccgagactggaggttgttcggggagcagggacgattag.

Amino acid sequence of the Rv2626c-RpfD fusion with the Ag85B signal sequence:

(SEQ ID NO: 96)
MTDVSRKIRAWGRRLMIGTAAAVVLPGLVGLAGGAATAGAFSMTTARDI
MNAGVTCVGEHETLTAAAQYMREHDIGALPICGDDDRLHGMLTDRDIVI
KGLAAGLDPNTATAGELARDSIYYVDANASIQEMLNVMEEHQVRRVPVI
SEHRLVGIVTEADIARHLPEHAIVQFVKAICSPMALASMTPGLLTTAGA
GRPRDRCARIVCTVFIETAVVATMFVALLGLSTISSKADDIDWDAIAQC
ESGGNWAANTGNGLYGGLQISQATWDSNGGVGSPAAASPQQQIEVADNI
MKTQGPGAWPKCSSCSQGDAPLGSLTHILTFLAAETGGCSGSRDD.

DNA sequence of the Rv2626c-RpfD fusion with the 19 kDa signal sequence:

(SEQ ID NO: 97)
Atgaagcgtggactgacggtcgcggtagccggagccgccattctggtcg
caggtctttccggatgttcaagcaacaagtcgactacaggaagcggtga
gaccacgaccgcggcaggtaccacggcaagccccggcatgaccaccgca
cgcgacatcatgaacgcaggtgtgacctgtgttggcgaacacgagacgc
taaccgctgccgctcaatacatgcgtgagcacgacatcggcgcgttgcc
gatctgcggggacgacgaccggctgcacggcatgctcaccgaccgcgac
attgtgatcaaaggcctggctgcgggcctagacccgaataccgccacgg
ctggcgagttggcccgggacagcatctactacgtcgatgcgaacgcaag
catccaggagatgctcaacgtcatggaagaacatcaggtccgccgtgtt
ccggtcatctcagagcaccgcttggtcggaatcgtcaccgaagccgaca
tcgcccgacacctgcccgagcacgccattgtgcagttcgtcaaggcaat
ctgctcgcccatggccctcgccagcatgacaccgggtttgcttactact
gcgggtgctggccgaccacgtgacaggtgcgccaggatcgtatgcacgg
tgttcatcgaaaccgccgttgtcgcgaccatgtttgtcgcgttgttggg
tctgtccaccatcagctcgaaagccgacgacatcgattgggacgccatc
gcgcaatgcgaatccggcggcaattgggcggccaacaccggtaacgggt
tatacggtggtctgcagatcagccaggcgacgtgggattccaacggtgg
tgtcgggtcgccggcggccgcgagtccccagcaacagatcgaggtcgca
gacaacattatgaaaacccaaggcccgggtgcgtggccgaaatgtagtt
cttgtagtcagggagacgcaccgctgggctcgctcacccacatcctgac
gttcctcgcggccgagactggaggttgttcggggagcagggacgatta
g.

Amino acid sequence of the Rv2626c-RpfD fusion with the 19 kDa signal sequence:

(SEQ ID NO: 98)
MKRGLTVAVAGAAILVAGLSGCSSNKSTTGSGETTTAAGTTASPGMTTA
RDIMNAGVTCVGEHETLTAAAQYMREHDIGALPICGDDDRLHGMLTDRD
IVIKGLAAGLDPNTATAGELARDSIYYVDANASIQEMLNVMEEHQVRRV
PVISEHRLVGIVTEADIARHLPEHAIVQFVKAICSPMALASMTPGLLTT
AGAGRPRDRCARIVCTVFIETAVVATMFVALLGLSTISSKADDIDWDAI
AQCESGGNWAANTGNGLYGGLQISQATWDSNGGVGSPAAASPQQQIEVA
DNIMKTQGPGAWPKCSSCSQGDAPLGSLTHILTFLAAETGGCSGSRDD.

### Example 3: Cloning and Overexpression of Fusion Proteins of the Cassette and Variants

### Preparation of the Antigen Cassette and its Variants as Fusion Protein Required a Modified Strategy Outline Below

Cloning: Multiple recombinant fusion proteins were created, of which two are exemplified here: one with four Mtb antigens (ESAT6-Rv1733c-Rv2626c-RpfD), and one with five antigens (Ag85B-ESAT6-Rv1733c-Rv2626c-RpfD). The genes encoding the protein antigens were PCR amplified using the primers in Table 5 and cloned into the pET28b vector (Novagen) via the indicated restriction enzyme sites. ESAT6 was PCR amplified from Mtb and first cloned into the pET23b vector (Novagen). It was subsequently

PCR amplified and cloned into pET28b. The genes for antigen 85B, Rv1733c, Rv2626c, and RpfD were all synthesized with their codons optimized for expression in E. coli (DNA2.0). Ag85B, and rpfD were synthesized without the bases encoding the N-terminal signal sequence, and rpfB was PCR amplified from Mtb without the N-terminal signal sequence.

The codon optimized genes were PCR amplified and cloned into pET28b creating N-terminal 6xHis-tagged fusion proteins. The genes were cloned with no spacer sequences, only the restriction enzyme sites between each gene. To remove the 2 transmembrane regions of Rv1733c, it was PCR amplified in 3 pieces which were ligated together. In another embodiment the 4 Ag and 5 Ag proteins were constructed with wild type Rv1733c including the transmembrane regions. The pET28b constructs were cloned in E. coli cloning strains, screened by restriction digest and sequenced to verify each construct. The DNA and amino acid sequences of the 4 Ag and 5 Ag fusions were prepared without the transmembrane regions of Rv1733c. Later versions of these fusions replaced RpfD with RpfB in the 4 Ag fusion, with RpfB placed either at the 5′ or the 3′ end of the fusion.

Expression: The plasmids encoding Construct D and its variant fusion proteins were transformed into E. coli T7 express (NEB) or E. coli BL21 DE3 (Novagen). Multiple colonies of each fusion construct were picked and grown overnight shaking at 37° C. in Tryptic Soy Broth (TSB) (Sigma). Overnight cultures were diluted 1:100 in TSB and grown shaking at 37° C. to OD600=0.6. Cultures were induced with 1 mM IPTG and grown shaking at 37° C. for 3 hours. Induced and uninduced aliquots of each culture were run on 4-12% Bis/Tris SDS-PAGE gels to verify induction of the fusion proteins. Colonies expressing each of the fusion proteins were frozen in TSB+20% glycerol at −80° C. as research stocks.

### Purification of Fusion Proteins

10 ml cultures were inoculated from glycerol stocks of the BE1726D and its variant fusion constructs and grown overnight shaking at 37° C. The overnight cultures were diluted 1:100 in 250 ml TSB and grown shaking at 37° C. to OD600=0.6. Cultures were induced with 1 mM IPTG and grown shaking at 37ºC for 3 hours. An aliquot of the induced sample was run on a 4-12% Bis/Tris SDS-PAGE gel to confirm induction of the protein. The induced culture was centrifuged at 6,000× g for 10 m and pellets were frozen at −80° C. Pellets were thawed and resuspended in 10 ml BPER buffer (Thermo Scientific), and an aliquot was taken for testing (lysate). Lysozyme (20 u/ml) and DNase I (25 U/ml) was added to help complete cell lysis. The lysed cells were centrifuged at 12,000× g for 10 minutes and the supernatant was collected (soluble fraction). The insoluble pellet was resuspended in 10 ml BPER buffer and a 100 μl aliquot was removed (insoluble fraction). The cells in the resuspended pellet were diluted with 10 ml 10% BPER buffer and the suspension was centrifuged at 12,000× g for 10 minutes. The supernatant was discarded and the pellet was washed again with 10 ml 10% BPER buffer 3 more times. The lysate, soluble and insoluble fractions and washes were run on a 4-12% Bis/Tris SDS-PAGE gel to confirm expression and determine the subcellular localization of the protein. The fusion proteins were found localized to the insoluble pellet in inclusion bodies. The insoluble pellet was resuspended in 10 ml denaturing binding buffer (DBB) (8 M urea, 92 mM Na₂HPO₄, 7 mM NaH₂PO₄, 10 mM Tris) pH 7.8. The inclusion bodies were lysed by sonication, and the lysate was cleared of debris by centrifugation at 12,000× g for 20 minutes.

Proteins with the transmembrane regions of Rv1733c deleted were purified by column purification. Five (5) ml of HisPur Cobalt resin (Thermo Scientific) was equilibrated 10) with DBB and incubated with 5 ml of cleared lysate. The mixture was rocked at room temperature for 90 minutes. The lysate/resin mixture was then loaded on a 30 ml column and washed with 25 volumes of denaturing wash buffer (8 M urea, 25 mM Na₂HPO₄, 75 mM NaH₂PO₄, 10 mM Tris, 12 mM sodium deoxycholate, pH 7.8). His-tagged protein was eluted from the Co+column by eluting with elution buffer (8 M urea, 10 mM Tris, 5% glycerol) pH 8.0 with 50, 100, 350, 500, and 1000 mM imidazole. Eluted proteins were run on a 4-12% Bis/Tris SDS-PAGE gel and clean fractions were dialyzed stepwise from 8M urea, 10 mM Tris, 5% glycerol to 10 mM Tris, 5% glycerol. Dialyzed protein was analyzed by SDS-PAGE for purity, western blot for the presence of each antigen, and was assayed for the presence of residual endotoxin. Pure samples with <0.25 U endotoxin/ml were aliquoted and frozen at 20 -80° C.

Proteins which have wild type Rv1733c were purified using an AKTA purifier (GE Healthcare). After the inclusion bodies were separated by BPER washes as above, the insoluble pellet was resuspended in 10 ml denaturing binding buffer (DBB)+20mM imidazole. The inclusion bodies were lysed by sonication, and the lysate was cleared of debris by centrifugation at 12,000× g for 20 minutes. Five (5) ml of Ni Sepharose High Performance (GE Healthcare) resin was equilibrated with DBB and incubated with 10 ml of cleared lysate. The mixture was rocked at room temperature for 2 hours. The mixture was then loaded on the AKTA purifier. All the lines used on AKTA purifier were equilibrated with DBB+20 mM imidazole, denaturing wash buffer (8 M urea, 25 mM Na₂HPO₄, 75 mM NaH₂PO₄, 10 mM Tris, 12 mM sodium deoxycholate, 20mM imidazole) pH 7.8, or elution buffer (8 M urea, 10 mM Tris, 5% glycerol, 20 mM imidazole), as needed. Proteins were eluted by gradient elution (elution buffer 1: 8 M urea, 10 mM Tris, 5% glycerol, 20 mM imidazole, run from 100% to 0; elution buffer 2: 8 M urea, 10 mM Tris, 5% glycerol, 500 mM imidazole, fun from 0 to 100%) and fractions were collected. The positive fractions were run on a 4-12% Bis-Tris SDS-PAGE gel and were dialyzed stepwise from 8 M urea, 10 mM Tris, 5% glycerol to 10 mM Tris, 5% glycerol. Dialyzed protein was analyzed by SDS-PAGE for purity, western blot for the presence of each antigen, and was assayed for the presence of residual endotoxin. Pure samples with <0.25 U endotoxin/ml were aliquoted and frozen at −80° C.

The foregoing describes the purification of the 4 Ag and 5 Ag proteins that were expressed with 6xHis tags. The proteins can also be expressed without tags. The untagged proteins can be purified by combining chromatographic methods including ion exchange and size exclusion chromatography and filtration methods such as tangential flow.

### Example 4: Immunogenicity of the 5 Ag and 4 Ag Fusion Proteins in Mice

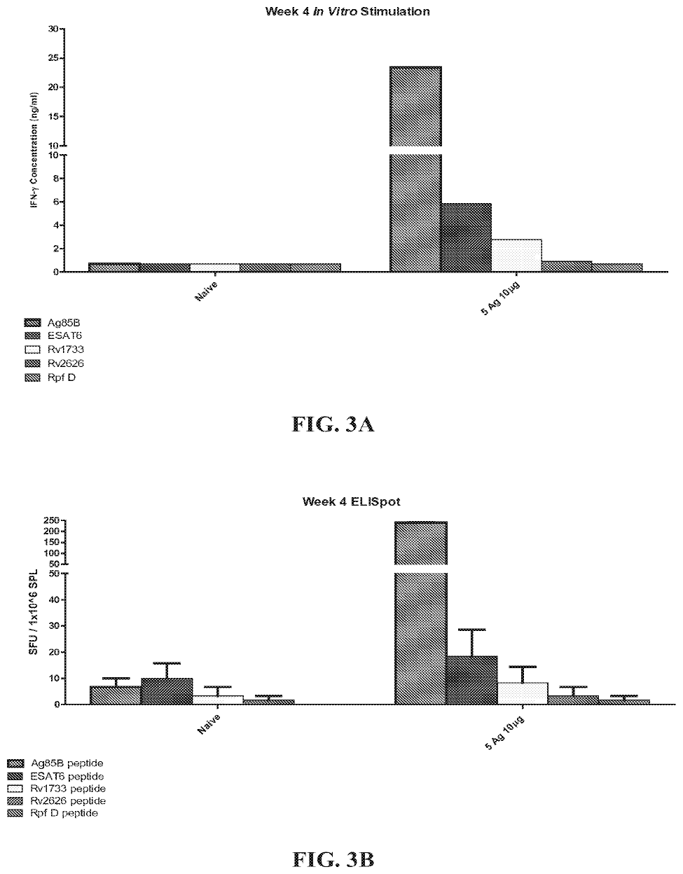

The 5 Ag fusion protein was tested for immunogenicity in CB6F1 mice, adjuvanted with a synthetic poly I:C TLR3 agonist. Multiple other adjuvants, such as TLR4 agonists, were tested and shown to be immunogenic, and thus the embodiment is independent of the adjuvant used, and applicable to many classes of adjuvants. Mice were immunized subcutaneously twice, two weeks apart, with 1 or 10 μg of adjuvanted fusion protein. Two (2) weeks after the second immunization, the mice were sacrificed and splenocytes were isolated. The splenocytes were incubated with recombinant protein antigens for in vitro stimulation and recombinant protein or overlapping peptides for ELISpot analysis. The 5 Ag fusion protein induced significant IFN-y responses to each antigen that were measureable by both in vitro stimulation and ELISpot (see, FIGS. 3A and 3B). The response to Ag85B, the most immunogenic and first antigen of the fusion protein, was much stronger to the responses to the other antigens.

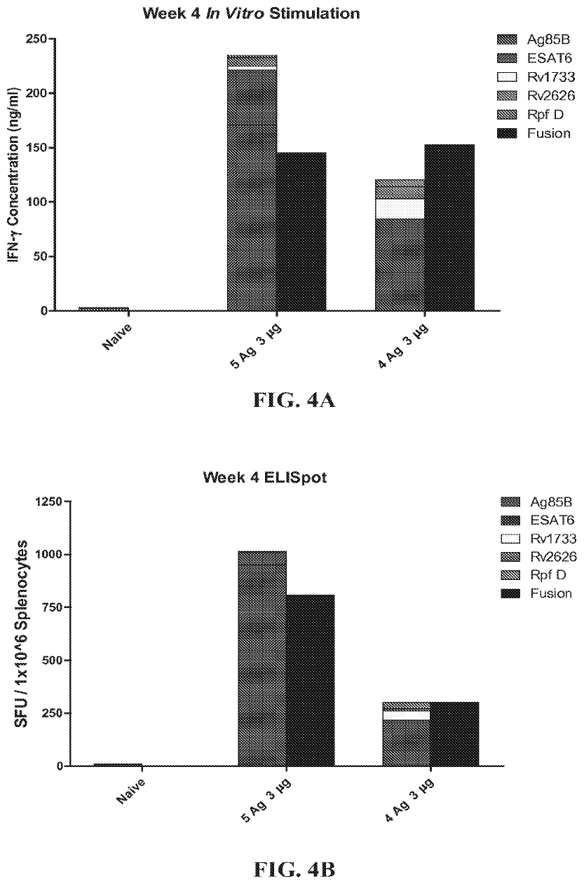

The 4 Ag and 5 Ag proteins were then both tested for immunogenicity in CB6F1 mice, adjuvanted with a synthetic MPL TLR4 agonist. Mice were immunized subcutaneously twice, two weeks apart, with 3 μg adjuvanted fusion protein. Splenocytes were isolated for in vitro stimulation and ELISpot. Splenocytes were stimulated with individual antigens or fusion proteins. Immunization with either the 4 Ag or 5 Ag fusion proteins induced IFN-γ responses to all antigens. Responses to ESAT6, Rv1733c, Rv2626c, and RpfD were all higher in the 4 Ag fusion protein, which lacks Ag85B, than the 5 Ag fusion protein (see, FIGS. 4A and 4B).

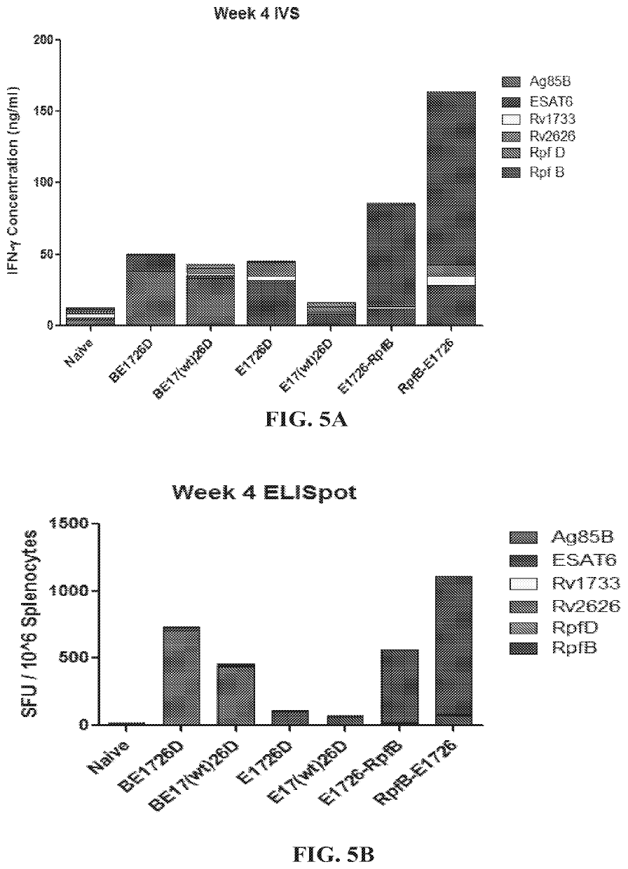

Immunogenicity studies were also performed on the fusion proteins with the wild-type Rv1733c (85B-ESAT6-Rv1733cwt-Rv2626c-RpfD and ESAT6-Rv1733cwt-Rv2626c-RpfD) and the 4 Ag fusions where RpfD was replaced by RpfB (ESAT6-Rv1733c-Rv2626c-RpfB and RpfB-ESAT6-Rv1733c-Rv2626c). These studies compared the immunogenicity of fusion proteins containing the modified Rv1733c with that of fusions containing the wild-type Rv1733c, and also the immunogenicity of fusion proteins containing RpfD with that of fusions containing RpfB. The studies showed that while replacing the modified Rv1733c with the wild-type 1733c did not affect overall immunogenicity, RpfB is significantly more immunogenic than RpfD in these fusions (see, FIGS. 5A and 5B).

### Example 5: Ongoing Protective Efficacy

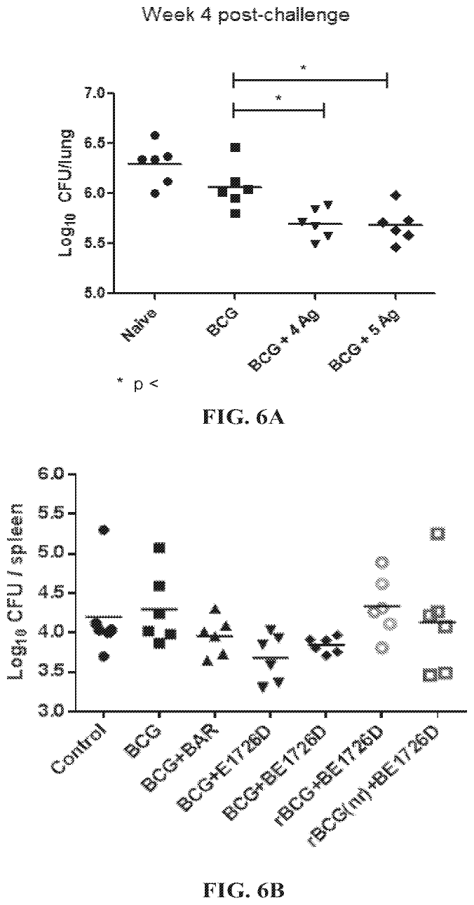

The 5 Ag and 4 Ag fusion proteins (BE1726D, E1726D) were used in a prime boost protection experiment in mice. Mice were primed with BCG SSI and recombinant BCG SSI overexpressing the 5 Mtb antigens that make up the 5 Ag fusion protein. Six (6) and 8 weeks later, the mice were boosted with either the 5 Ag or the 4 Ag protein plus a poly I:C adjuvant. Four (4) weeks after the second boost mice received an aerosol Mtb challenge of 50-100 CFU. Mice were then sacrificed at 4 and 12 weeks post challenge to determine viable bacteria in the lungs (see, FIG. 6A) and spleen (see, FIG. 6B). This experiment determined whether the large response to Ag85B is more protective in mice than the more broad response to the other 4 antigens in the 4 Ag fusion protein.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1-10. (canceled)

11. A composition comprising at least four Mycobacterium tuberculosis (Mtb) antigens, wherein the composition comprises:
at least one acute Mtb antigen, at least one latent Mtb antigen, and at least one resuscitation Mtb antigen; or
at least two latent Mtb antigens, and at least one resuscitation Mtb antigen;
wherein the composition comprises at least one nucleic acid molecule encoding at least one of the Mtb antigens.

12. The composition according to claim 11 wherein at least two Mtb antigens are encoded by one or more nucleic acid molecules within one or more vectors.

13. The composition according to claim 12 wherein the one or more vectors are selected from adenovirus 4, adenovirus 5, chimpanzee adenovirus 3, chimpanzee adenovirus 63, chimpanzee adenovirus 68, MVA, PIV2, PIV3, or hPIV2.

14. The composition according to claim 11 wherein the at least two Mtb antigens are encoded by a single nucleic acid molecule within the same vector as a fusion protein.

15. The composition according to claim 11 wherein the acute Mtb antigen is Ag85B, ESAT6, MPT64, PPE15, PPE51, or Rv3615c; the latent Mtb antigen is Rv1733c, Rv1733cΔTM, Rv2626c, Rv3407, or Rv2628c; and the resuscitation Mtb antigen is RpfB, RpfD, or RpfE.

16. (canceled)

17. The composition according to claim 11 which comprises one or more of:
ESAT6, Rv1733c, Rv2626c, and RpfD Mtb antigens;
ESAT6, Rv1733c, Rv2626c, and RpfB Mtb antigens;
RpfB, ESAT6, Rv1733c, and Rv2626c Mtb antigens;
Ag85B, ESAT6, Rv1733c, Rv2626c, and RpfD Mtb antigens;
Ag85B, ESAT6, Rv1733c, Rv2626c, and RpfB Mtb antigens;
PPE51, Rv1733c, Rv2628c, and RpfD Mtb antigens;
PPE51, Rv1733c, Rv2628c, and RpfB Mtb antigens;
Rv3407, Rv1733c, Rv2626c, and RpfB Mtb antigens; or
Rv3407, Rv1733c, Rv2626c, and RpfD Mtb antigens;
wherein the composition comprises at least one nucleic acid molecule encoding at least one of the Mtb antigens.

18. The composition according to claim 11 further comprising a pharmaceutically acceptable carrier.

19. (canceled)

20. A method of eliciting an immune response against Mycobacterium tuberculosis in a mammal comprising administering to the mammal an immunologically sufficient amount of the composition according to claim 11.

21. The composition according to claim 11, wherein the composition comprises at least the four Mycobacterium tuberculosis (Mtb) antigens ESAT6, Rv1733c, Rv2626c, and RfpB, wherein the at least four Mtb antigens are encoded by a single nucleic acid molecule as a fusion protein within the same vector, optionally wherein the composition further comprises the antigen Ag85B and the fusion protein has the sequence Ag85B-ESAT6-Rv1733c-Rv2626c-RpfB.

22. The composition according to claim 21, wherein the first and/or second transmembrane region of Rv1733c is deleted.

23. The composition according to claim 21, further comprising a pharmaceutically acceptable carrier.

24. A method of eliciting an immune response against Mycobacterium tuberculosis in a mammal comprising administering to the mammal an immunologically sufficient amount of the composition according to claim 21.

25. The composition according to claim 11, wherein the composition comprises at least the four Mycobacterium tuberculosis (Mtb) antigens ESAT6, Rv1733c, Rv2626c, and RpfD, wherein the at least four Mtb antigens are encoded by a single nucleic acid molecule as a fusion protein within the same vector.

26. The composition according to claim 25, wherein the first and/or second transmembrane region of Rv1733c is deleted.

27. The composition according to claim 25, wherein the composition further comprises the Ag85B Mtb antigen.

28. The composition according to claim 25, wherein the composition further comprises a pharmaceutically acceptable carrier.

29. A method of eliciting an immune response against Mycobacterium tuberculosis in a mammal comprising administering to the mammal an immunologically sufficient amount of the composition according to claim 25.
